(19) United States
(12) Patent Application Publication
(10) Pub. No.: US 2025/0235703 A1
(43) Pub. Date: Jul. 24, 2025

(54) SYSTEMS AND METHODS FOR CLOSED-LOOP NEUROMODULATION TO TREAT PAIN OR RESTORE FUNCTION AFTER SPINAL CORD INJURIES

(71) Applicant: REGENTS OF THE UNIVERSITY OF MINNESOTA, Minneapolis, MN (US)

(72) Inventors: David Darrow, Minneapolis, MN (US); Theoden Netoff, Minneapolis, MN (US); Disa Sullivan, Minneapolis, MN (US); Seth König, Minneapolis, MN (US); Alexander Herman, Minneapolis, MN (US)

(21) Appl. No.: 19/033,333

(22) Filed: Jan. 21, 2025

Related U.S. Application Data
(60) Provisional application No. 63/623,032, filed on Jan. 19, 2024.
(60) Provisional application No. 63/623,138, filed on Jan. 19, 2024.

(51) Int. Cl. A61N 1/36; A61N 1/05

(52) U.S. Cl. CPC A61N 1/36139; A61N 1/05; A61N 1/3615; A61N 1/36185

(57) ABSTRACT

A neurostimulation system includes a plurality of stimulation electrodes, a plurality of recording electrodes, a waveform generator in electrical communication with the plurality of stimulation electrodes, and one or more computing devices in electrical communication with the plurality of recording electrodes and the waveform generator. The one or more computing devices cause the waveform generator to electrically excite the plurality of stimulation electrodes of the plurality of stimulation electrodes. After electrically exciting the stimulation electrodes, a neural signal is received from the plurality of recording electrodes. The neural signal is determined to be an electrically evoked compound action potential (“ECAP”) by determining one or more features of the neural signal. One or more stimulation parameters are determined based on one or more characteristics of the ECAP. The waveform generator is then caused to electrically excite the plurality of stimulation electrodes according to the one or more stimulation parameters.

### CROSS-REFERENCE TO RELATED APPLICATIONS

This application claims priority to U.S. Patent Application No. 63/623,032 filed Jan. 19, 2024, and entitled, “A Method and System for Closed-Loop Neuromodulation to Restore Function After Spinal Cord Injury,” and U.S. Patent Application No. 63/623,138 filed Jan. 19, 2024, and entitled, “A Method and System for Closed-Loop Neuromodulation to Restore Function After Spinal Cord Injury,” each of which is hereby incorporated by reference in its entirety.

### BACKGROUND

Recently, spinal cord stimulation (“SCS”), that is, electrically exciting the spinal cord, has successfully treated individuals that suffer from chronic pain. Successful spinal cord stimulation often utilizes closed-loop stimulation schemes, which rely on control signals. However, control signals that are noisy or are improperly classified can provide inadequate SCS and therefore inadequate treatment of pain Thus, it would be desirable to have improved systems and methods for closed-loop neuromodulation to treat pain or restore function after spinal cord injuries.

### SUMMARY OF THE DISCLOSURE

Some embodiments of the disclosure provide a neurostimulation system that includes a plurality of stimulation electrodes; a plurality of recording electrodes; a waveform generator in electrical communication with the plurality of stimulation electrodes; and one or more computing devices in electrical communication with the plurality of recording electrodes and the waveform generator. The one or more computing devices are configured to: cause the waveform generator to electrically excite the plurality of stimulation electrodes of the plurality of stimulation electrodes; after electrically exciting the two stimulation electrodes, receive, from the plurality of recording electrodes, a neural signal; determine that the neural signal is an electrically evoked compound action potential (“ECAP”); determine one or more stimulation parameters, based on one or more characteristics of the ECAP; and cause the waveform generator to electrically excite the plurality of stimulation electrodes according to the one or more stimulation parameters. The neural signal is determined to be an ECAP by determining one or more features of the neural signal and determining that the neural signal is an ECAP based on the one or more features of the neural signal.

It is another aspect of the present disclosure to provide a computer implemented method for classifying neural signals. The method includes providing, using one or more computing devices, a neural signal to a machine learning model that classifies the neural signal as an ECAP, a non-ECAP, or an outlier, the machine learning model being trained on training data comprising neural signals classified as ECAP, neural signals classified as non-ECAP, and neural signals classified as outliers; receiving, from the machine learning model and using the one or more computing devices, an output classifying the neural signal as an ECAP; and determining, using the one or more computing devices, that the neural signal is an ECAP based on the output from the machine learning model classifying the neural signal as an ECAP.

It is still another aspect of the present disclosure to provide a neurostimulation system that includes a plurality of stimulation electrodes; a plurality of recording electrodes; a waveform generator in electrical communication with the plurality of stimulation electrodes; and one or more computing devices in electrical communication with the plurality of recording electrodes. The one or more computing devices are configured to cause the waveform generator to electrically excite two stimulation electrodes of the plurality of stimulation electrodes; after electrically exciting the two stimulation electrodes, receive, from at least two recording electrodes of the plurality of recording electrodes, a neural signal; determine that the neural signal is an electrically evoked compound action potential (“ECAP”); remove a stimulation artifact from the neural signal that is an ECAP to yield a clean neural signal; determine one or more stimulation parameters, based on one or more characteristics of the clean neural signal; and cause the waveform generator to electrically excite the stimulation electrodes according to the one or more stimulation parameters.

It is yet another aspect of the present disclosure to provide a method of training a machine learning model. The method includes receiving neural signal data; generating feature data by extracting features from the neural signals in the neural signal data; generating classification data by classifying the neural signals in the neural signal data based on the feature data; and training a machine learning model on training data comprising pairs of matched classification data and neural signal data.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic illustration of a block diagram of a neurostimulation system.

FIG. 2 shows a schematic illustration of specific block diagrams of the components of the neurostimulation system of FIG. 1 including the electrodes, the waveform generator, the computing device, and the server.

FIG. 3 shows a flowchart of a process of cleaning neural signals (e.g., removing artifacts, and specifically stimulation artifacts from neural signals).

FIG. 4 shows a flowchart of a process of training a machine learning model (e.g., to classify a neural signal as an ECAP, a non-ECAP, or an outlier, to output one or more thresholds for each feature).

FIG. 5 shows a flowchart of a process of determining a polarity preference for a patient.

FIG. 6 shows a flowchart of a process of treating one or more conditions of a patient (e.g., using electrical stimulation, such as of the spinal cord).

FIG. 7 shows a flowchart of a process of determining whether a neural signal is an ECAP, which can be a specific implementation of a block of the process of FIG. 6.

FIG. 8 provides an experimental overview. FIG. 8A shows a stimulation setup where participants were implanted with two percutaneous Octrode™ leads near the thoracic region of the spinal cord. Externalized leads were connected to an Atlas NeuraLynx 192 recording system and stimulation was delivered using an Atlas Stim Headbox or a TDT system.

FIG. 8B shows stimulation trains, where they were delivered for 10 seconds at 38 Hz using asymmetric, charged-balanced, biphasic stimulation pulses with alternating polarity on contacts 7 & 8. FIG. 8C shows a graph where paresthesia intensity was measured during stimulation. Paresthesia intensity varied slightly from day to day (different lines) within a participant though paresthesia intensity consistently increased more rapidly for 350 S pulse widths (orange) than 150 μS 197 pulse widths (blue). FIG. 8D shows a graph of the average ESR signals from a single channel in the same participant as in FIG. 8C for stimulation amplitudes relative to perception threshold showing that an ECAP was not readily observed at perception threshold or detected at stimulation amplitudes just above perception threshold. *Approximate P1, N1, P2 times are labeled in FIGS. 8B & D.

FIG. 9 shows waveform preprocessing and feature extraction from a 6 mA stimulation. FIG. 9A shows a graph of Post-stimulation (0.375 to 8.0 ms) waveforms being clustered (different colors) in PCA space using k-means clustering and artifacts were fit for each cluster using an exponential fit. Typically, 1 cluster per stimulation polarity was found, but because of movement, respiratory, or heartbeat artifacts there were sometimes more clusters (black lines). FIG. 9B) shows artifact-cleaned waveforms for each cluster revealing ECAPs. Gray window 253 (0.375 to 2.375 ms) was the ECAP window used in feature extraction. FIG. 9C shows a graph of the distribution of AUC values from artifact-cleaned waveforms in FIG. 9B showing bimodal distribution presumably related to stimulation polarity. FIG. 9D shows waveforms sorted by AUC again showing bimodal distribution in AUC as well as potentially a small latency shift in the evoked responses presumably related to stimulation polarity.

FIG. 10 shows the building of an ECAPs classifier using PCA, k-means clustering, and a KNN classifier. FIG. 10A shows that PCA was used to extract nonparametric features from all evoked waveforms, and then k-means clustering was used for unsupervised grouping of waveforms. FIG. 10B shows that from the cluster's averaged waveforms, clusters were then labeled as representing non-ECAPs (blue), ECAPs (green), outliers (red) based P2P and the presence or absence of detectable peaks and valleys. FIG. 10C shows ECAPS and outliers were represented by several clusters each, while there was only 1 cluster representing non-ECAPS. FIG. 10D shows that mean evoked waveforms based KNN classifier labeled waveforms further divided into high- and low-confidence waveforms. FIG. 10E shows the mean variance of evoked waveforms based on a KNN classifier labeled waveforms clustered further divided into high- and low-confidence waveforms. FIG. 10F shows the proportion of waveforms classified by the KNN classifier as non-ECAPs, ECAPs, and outliers further divided into high- and low-confidence waveforms. FIGS. 10G and I show relative feature importance according to tree ensemble models using waveform classes determined by the KNN classifier for FIG. 10G all non-PCA features with all classes, for FIG. 10H all features with all classes, and for FIG. 10I for all non-PC features for non-ECAP and ECAP classes only. *PCA FIG. 10A was applied to all waveforms, while k-means clustering FIG. 10B-C and KNN classification FIG. 10D-F were applied within each outer loop; FIGS. 10B-F show example data from a single outer loop. FIGS. 10G-I show distribution of feature importance across outer loops.

FIG. 11 shows a logistic GLME model visualization of factors predicting outlier classification using optimal thresholds calculated from the ROC analysis. FIG. 11A shows a graph of the recording distance, FIG. 11B shows a graph of the stimulation amplitude, and FIG. 11C shows a graph of the stimulation pulse width, each of which were strong predictors of outlier classification while stimulation polarity FIG. 11D was not. All plots show median+/−IQR calculated across all other predicted values. The Logistic GLME model predicted that the large spread around the median in FIG. 11B was due to the increased susceptibility to artifacts at higher stimulation amplitudes on recording electrodes proximal to the stimulation sites.

FIG. 12 shows factors predicting ECAP classification using optimal thresholds calculated from the ROC analysis. FIG. 12A shows a graph of the average probability of detecting an ECAP for individual sessions across multiple channels separated by stimulation polarity and pulse width shows a lot of inter-participant and intra-participant variability. FIG. 12B shows a graph of the average probability of detecting an ECAP across all participants and sessions separated by stimulation polarity and pulse width. Note there is unequal sampling across stimulation parameters. FIG. 12C shows a GLME model fit to the data in B, which shows that stimulation polarity and pulse width affect the probability of detecting an ECAP. FIG. 12A shows a graph of the difference in AUC across stimulation polarity for each session (different shades) indicating the effects of preferred stimulation polarity on ECAP AUC. Note sessions could have different stimulation pulse widths. FIG. 12E shows a graph of the Updated GLME model predictions to the data using preferred stimulation polarity. FIG. 12F shows a graph of E50 from the GLME model using preferred stimulation polarity indicating that preferred stimulation polarity needs ˜1.25 mA less current to evoke an ECAP. FIG. 12G shows a graph of the observed average AUC relative to perception threshold, which showed an abrupt increase at stimulation amplitudes above perception particularly for the preferred stimulation polarity. FIG. 12H shows a graph of the probability of detecting an ECAP relative to perception threshold from participant 9 on channel 3 from a single day. FIG. 12I shows a graph of the GLME model fit to the data aligned to the perception threshold across all participants.

FIG. 13 shows factors predicting ECAP N1 Latency during individual sessions. FIG. 13 top row shows beta weight estimates from linear models for stimulation amplitude, stimulation polarity, ECAP AUC, and recording distance from the stimulation site. FIG. ### bottom row shows the same as top row but partial eta-squared (ηₚ²) indicating the effect size. Linear models were fit to individual recording sessions where all stimulation pulses had the same pulse width. Values in parentheses in titles are the median estimate across sessions. Each participant has a different shading.

FIG. 14 shows lead migration and post hoc electrode alignment based on N1 latency. FIG. 14A shows a schematic of method for post-hoc re-alignment using a simple regression method based on N1 latency. The relative rostral-caudal position of the right lead was programmatically adjusted, then the N1 latency against rostral-caudal (RC) position regressed, and then the MSE was calculated. The relative position of the right lead that minimized the MSE of the regression of N1 latency versus RC position was chosen. FIG. 14B shows averaged ECAP waveforms for the left lead (green, channels 2-6) and right lead (red, channels 10-16) for stimulation amplitudes that elicited ECAPs plotted with respect to their Rostral-Caudal (“RC”) location relative to stimulation electrodes 7/8. Peaks and valleys were detected with findpeaks in Matlab. Only channels with detectable peaks and valleys were included. Note for this participant channel 1 was used as a ground electrode. FIG. 14C shows averaged ECAP waveforms for contacts on the left (green) and right (red) leads using alignment based on postoperative X-ray positions. FIG. 14D shows averaged ECAP waveforms for contacts on the left (green) and right (red) leads using alignment based on N1-latency corrected position. The peak and valley times and amplitudes match better for the N1-latency corrected alignment than for the postoperative X-ray based alignment. FIG. 14E shows N1 latency corrected times plotted against their relative RC position. The slope (m) indicates the estimated conduction velocity of the ECAPS based on the N1 latency as a function of position. FIG. 14F shows N1 conduction velocity across recording sessions and participants (different shading). FIG. 14I shows predicted lead migration across sessions and participants (same shading as H) based on N1 latency corrected position. Note, estimated lead migration appeared relatively stable across sessions. Predicted lead migration is only based on relative lead migration between left (contacts 1-6) and right (contacts 10-16) leads and cannot be used to predict absolute lead migration relative to the spinal cord. Also, note, conduction velocity estimates using this methodology were higher than the formal analysis likely due to distortion of the ECAPs waveforms when averaging across stimulation polarity and the inclusion of contacts close to the stimulation sites (e.g., contact 6).

FIG. 15 shows visualization of PCA and correlation of PCA scores with waveform features for the same example outer loop data in FIG. 15. FIGS. 15 A and B show average waveform characteristics as indicated by PCA (A) and waveform amplitude (B) varied greatly across clusters determined by k-means clustering. Note, waveform P2P amplitude was not used during clustering but only to label clusters. FIGS. 15C-E) show visualization of the 1st 3 PC by taking the average of waveforms sorted by every 10th-percentile for each PC score. FIGS. ###F-H show average correlation (r) of the 1st 3 PC with waveform features. While PC1 was strongly correlated with average voltage and average slope, PC2 and PC3 appeared correlated with various peaks and valleys instead of any particular waveform feature.

FIG. 16 shows a distribution of waveform features and PC scores by KNN classifier results for the same example outer loop data in FIG. 10. Distribution of different waveform features and PC scores separated by waveform class and low vs high-confidence classification. The magnitude (∥) of the average voltage, average slope, and PC scores was used to create one tailed distributions; these distributions appeared symmetric when using raw values.

FIG. 17 shows the effect of stimulation parameters on waveform features. All plots median+/−IQR calculated across all other predicted values. Data does not include outliers, and the number of observations is not equal across participants. For N1 amplitude, only waveforms with a detectable N1 were used.

FIG. 18 shows an example averaged evoked responses across participants. Example average evoked artifact-cleaned waveforms for each participant on an example channel for each stimulation polarity (warmer vs cooler colors) across different stimulation amplitudes. Other than removing the artifact and averaging, no other processing was done.

FIG. 19 shows the probability of detecting an ECAP relative to perception threshold. FIG. 19A shows the probability of detecting an ECAP for each participant on their most sensitive channel for each pulse width and for preferred versus non-preferred polarity. The probability of detecting an ECAP for preferred polarity was consistently closer to the perception threshold than the non-preferred polarity. Additionally, in all participants, ECAPs were only detected above the perception threshold. FIG. 19B shows an example averaged evoked responses for Participant 9 on channels 2 and 3 at different stimulation amplitudes relative to perception threshold. ECAPs were clearly observed at 4 mA above the perception threshold and not at 2 mA below perception threshold. At perception threshold small deviations on channel 2 (P2P˜5.5 μV) and channel 3 (P2P˜7.7 μV) were observed. However, individual waveforms were not classified as ECAPs. Furthermore, artifacts remnants, especially on channel 3, indicate that at least some parts of these waveforms may be artifacts.

FIG. 20 shows the experiment layout. FIG. 20(A) shows two Octrode™ leads with a total of 16 electrodes were implanted. The stimulation waveform was delivered to electrodes 7 and 8 as asymmetric biphasic pulses at 38 Hz. The recording electrodes were connected to a Neuralynx or a TDT device. FIG. 20B shows an example X-ray from participant #7. Ref is the recording reference electrode. Stim is the stimulator.

FIG. 21 shows the artifacts are the same size in the normalized recordings. This dataset is from ch(11)-ch(12) in participant #6 in the second visit during cathode-on-8 stimulation at different stimulation amplitudes. The mean recordings are FIG. 21(A) un-normalized and FIG. 21(B) normalized by the stimulation amplitude. FIG. 21(C) shows small ECAPs ride on huge stimulation artifact. The normalized artifacts are very similar between different stimulation amplitudes, reflecting a linear response. This motivated the new S(AND) method to remove the artifacts in spinal-cord ECAPs.

FIG. 22 shows the novel artifact removal SAND method using spatial and amplitude differences is illustrated in steps. This dataset is Ch(11)-Ch(12) in participant #6 in the second visit during cathode-on-8 stimulation. FIG. 22(A) shows the mean recorded waveforms from super- and sub-threshold stimulation amplitudes (10 mA and 2 mA, the black and blue lines, respectively). The inset shows ECAPs, at the super-threshold amplitude, riding on an artifact in the time window 0-3 ms. FIG. 22(B) shows the waveforms were normalized by their respective stimulation amplitudes and the difference was taken (the red line). Note the reduction in the artifact amplitude in the difference (red line). FIG. 22(C) shows a zoomed view of the differential signal in the time window 0-3 ms shows clean SAND ECAPs without artifacts. Note this SAND ECAP have the units of uV/mA, which will be further multiplied by the large stimulation amplitude (10 mA in this case) to transform the units to uV.

FIG. 23 shows a stimulus artifact using the double-exponential method (exp2, blue lines) versus the novel SAND method (red lines) on Ch(11)-Ch(12) in participant #2's first visit. The two columns represent the two stimulation polarities. The rows represent stimulation amplitudes (suprathreshold: 6 and 5 mA; and sub-threshold: 2 mA). The SAND method used the 1-mA stimulation response to remove the artifacts for all stimulation amplitudes.

FIG. 24 shows dose-response curves of multiple channels in one participant (#3, visit 2) for cathode-on-8 stimulation. The inset shows the definition of ECAP metrics: peak-to-peak (P2P) and area under the curve (AUC). The dose response curves include P2P (top row) and AUC (bottom row) when artifact removal was done using the double exponential curve fit (exp2, left column) versus the novel SAND method (right column). The channels shown here are the good channels with ECAPs in this dataset, determined by an investigator. The SAND method used the 1-mA stimulation response to remove the artifacts for all stimulation amplitudes.

FIG. 25 shows peak-to-peak (P2P) dose-response curves of Ch(2)-Ch(3) in all participants (P in subplot titles) and visits (v-x in subplot titles) during cathode-on-8 stimulation. The blue curves represent ECAPs processed using the double exponential (exp2) artifact removal method, while the red curves are for the SAND method. ECAP thresholds (ET) are shown as black diamonds for exp2 and orange triangles for SAND. The SAND method used the 1-mA stimulation response to remove the artifacts for all stimulation amplitudes.

FIG. 26 shows on average the ECAP threshold (ET) is greater than the perception threshold (PT). ETs were calculated using peak-to-peak (P2P, top row) or area-under-the curve (AUC, bottom row). The columns are the combination of the two stimulation polarities (cathode on 8 and cathode on 7) and the two artifact-removal methods (exp2 and SAND). The dots' colors represent a participant/visit combination. The ETs of the same color are for different channels (see FIG. 5); and the corresponding PT is randomly scattered in x-axis (using MATLAB's swarmchart) around the patient reported value, depicted as a vertical line with the same color where the line spans the whole range of detected ETs on the y-axis. The gray lines are the best-fit lines to the data with coefficients shown in each subplot. The dashed line is a visual reference line with 1:1 correspondence between the two axes.

FIG. 27 shows the correlation matrix among the ECAP thresholds (ET) and participants' reported perception thresholds (PT). FIG. 27(A) shows an illustration of the data used to construct the correlation matrix. FIG. 27(B) shows the ETs were determined under three conditions: (c1) the ECAP metric being either peak-to-peak (P2P ET) or area under the curve (AUC ET); (c2) the artifact removal method being either double exponential (exp2) or SAND; and (c3) the stimulation polarity being either cathode on 8 or cathode on 7. FIG. 27(C) shows the correlation matrix. The upper diagonal contains correlations between pairs of conditions c1 and c2 (e.g., P2P ET of exp2 and P2P ET of SAND) while fixing condition c3 to cathode on 7. The lower triangle is similarly structured but with fixing condition c3 to cathode on 8. The diagonal contains correlations between pairs of condition c3 while fixing conditions c1 and c2. All correlations have p<1E-13. *The correlation between PTs under the two stimulation polarities excluded data from patients 1, 2 and 8, where PT was measured for a stimulation train switching polarity per pulse. Hence, no polarity-wise PT was available.

FIG. 28 illustrates an example of how ECAPs can be captured on a SCS lead.

FIG. 29 illustrates an example of SCS lead placement for chronic pain vs SCI and stimulation configuration on a paddle lead.

FIG. 30 illustrates an example of ECAP responses across stimulation amplitude recorded on a paddle lead placed in the lumbar spinal cord for SCI.

FIG. 31 illustrates an example of how ECAP morphology and features, from a single channel, emerge across stimulation amplitude.

FIG. 32 illustrates an example of ECAP responses across time and statistically significant clusters of time identifying morphology/time windows of interest.

FIG. 33 illustrates an example of bipolar subtracted referenced ECAP responses from an SCS paddle, with the title indicating which electrodes were referenced.

FIG. 34 illustrates an example of ECAP responses across stimulation amplitude recorded on leads placed in the thoracic spinal cord for chronic pain.

FIG. 35 illustrates an example of an electromyography (EMG) and accelerometer recording setup and how muscle activity and volitional movement improves with spinal cord stimulation in a participant receiving SCS for SCI.

FIG. 36 illustrates an example illustrating the effect of SCS and effort on maximal force on a volitional biking task.

FIG. 37 illustrates an example of how SCS impacts spasticity, quantified by the Modified Ashworth Scale, over time.

FIG. 38 illustrates an example of how SCS impacts systolic blood pressure.

FIG. 39 illustrates an example of how SCS impacts muscle synergies.

FIG. 40 illustrates an example of how SCS reduces the number of spasms over time.

FIG. 41 illustrates an example of how SCS improves bowel function over time.

FIG. 42 illustrates an example of how SCS improves female sexual function over time.

FIG. 43 illustrates an example ECAP response across increasing stimulation amplitudes of lumbar SCS in a participant with SCI.

FIG. 44 illustrates an example of statistically significant timepoints on ECAP waveform accounting for different features in a generalized linear mixed-effects (GLME) model.

FIG. 45 illustrates an example of resulting waveforms when ECAPs above and below the motor threshold are subtracted from each other.

FIG. 46 illustrates an example of overall EMG power improving with SCS turned on.

FIG. 47 illustrates examples of how EMG and IMU metrics capture different components of muscle synergies improvement with SCS.

FIG. 48 illustrates examples of how EMG and IMU metrics capture different components of muscle power improvement with SCS.

FIG. 49 illustrates an example of ECAP response across increasing stimulation amplitudes of lumbar SCS in a participant with SCI.

### DETAILED DESCRIPTION OF THE PRESENT DISCLOSURE

SCS (e.g., to treat chronic pain) typically relies on a closed loop control scheme. In this closed loop control scheme, a specific neural signal, an electrically evoked compound action potential (“ECAP”), is used as the control signal for closed loop feedback. More specifically, the peak to peak amplitude (“P2P”) of the ECAP is used as the control signal, where the amplitude of the next electrical stimulation of the spinal cord is determined by the previous P2P amplitude of the ECAP. In this way, the following processes can proceed continuously, as follows: electrical stimulation, followed by ECAP sensing and analysis, followed by electrical stimulation adjusted from the ECAP, and so on.

While ECAPs have been successful control signals in SCS, sometimes ECAPs improperly identified as ECAPs (e.g., a false positive). In other words, a non-ECAP signal can be misidentified as an ECAP and used as the control signal for SCS. This can provide inadequate SCS, that is, a stimulation amplitude that is either too high or too low. In some cases, this can at least temporarily throw off the closed-loop control of the system (e.g., until an actual ECAP is used as the control signal from subsequent stimulation) leading to pain for the patient. Further, aside from misidentification of non-ECAPs used as control signals, sometimes ECAPs although utilized properly, can suffer from stimulation artifacts. Similarly to using non-ECAPs as a control signal, artifact plagued ECAPs can result in control signals that are inadequate or less than ideal (e.g., the P2P as the control signal is less accurate with an artifact plagued ECAP).

Some embodiments of the disclosure provide advantages to these issues (and others) by providing improved systems and methods for closed-loop neuromodulation to treat pain or restore function after spinal cord injuries. For example, some embodiments of the disclosure provide methods to effectively classify ECAPs (e.g., from ECAPs, non-ECAPs, and outliers) to ensure that ECAPs (and not other signals) are used as the control signal for closed-loop SCS. As another example, some embodiments of the disclosure provide methods to effectively remove artifacts (e.g., stimulation artifacts) from neural signals, including ECAPs, to provide a higher quality ECAP as the control signal (e.g., during closed-loop SCS).

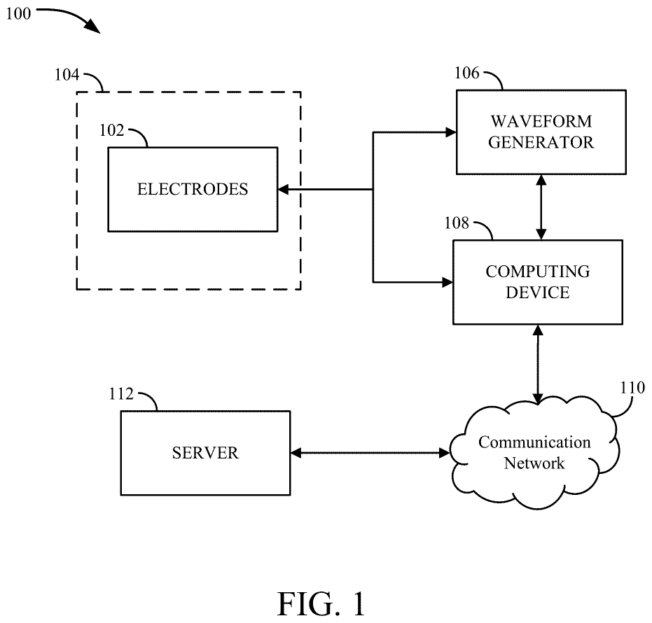

FIG. 1 shows a schematic illustration of a block diagram of a neurostimulation system 100. The neurostimulation system 100 can include electrodes 102 at least partially implanted within a patient 104, a waveform generator 106, a computing device 108, and a server 112. As described below, the electrodes 102 can include one or more pairs of stimulation electrodes, one or more pairs of recording electrodes, and one or more pairs of reference electrodes. The electrodes 102 can be implanted within a patient to contact the spinal cord of the patient. For example, in a patient within a spinal cord injury (e.g., an incomplete or a complete spinal cord injury), some or all of the electrodes 102 can be positioned below or at the injury site (e.g., where the spinal cord is partially or completely severed). In other words, some or all of the electrodes 102 can be caudal to the injury site. In some cases, some or all of the electrodes can be located on one or more leads. For example, where there are multiple leads (e.g., two leads), each lead can include a plurality of electrodes (e.g., of the electrodes 102). In some cases, each lead can extend along a length of a spinal cord, where each electrode is located at a different position along the spinal cord. In some embodiments, some or all of the stimulation electrodes can be positioned below (or more caudal relative to) the recording electrodes.

As shown in FIG. 1, the waveform generator 106 can be in electrical communication with the electrodes 102. More specially, the waveform generator 106 can be in electrical communication with the stimulation electrodes of the electrodes 102 to provide a stimulation waveform, signal, etc., to the stimulation electrodes (e.g., to electrically excite the spinal cord, where each of the stimulation electrodes can be in contact with the spinal cord of a patient). The waveform, signal, etc., can be a pulse (e.g., a rectangular pulse, a square pulse, etc.) in the time domain. Accordingly, the waveform generator 106 can be a pulse generator. In some cases, the waveform generator 106 can reverse a polarity of the stimulation electrodes, such as by inverting the stimulation waveform. The computing device 108 can be in electrical communication with the electrodes 102 and the waveform generator 106. In some cases, the computing device 108 can be in electrical communication with electrodes 102, specifically, the recording electrodes (and the reference electrodes) of the electrodes 102. In this way, neural signals (or neural signal data) can be acquired by two recording electrodes, a recording electrode and a reference electrode, etc.

In some cases, the neurostimulation system 100 can include a server 112. The server 112 can be in communication with the computing device 108 (and the waveform generator 106), such as via a communication network 110. In some configurations, the server 112 can provide data, computational power, etc., to the computing device 108 and the waveform generator 106. Although FIG. 1 shows the waveform generator 106 and the computing device 108 being different components (e.g., standalone components), the waveform generator 106 and the computing device 108 can be a single component (e.g., with the waveform generator 106 including the computing device 108 or vice versa). In some cases, although one computing device 108 has been shown in FIG. 1, the neuromodulation system 100 can include multiple computing devices 108, which can implement some or all of the processes described herein.

As shown in FIG. 1, at least a portion of the electrodes 102 can be implanted in the patient 104. Further, the waveform generator 106, the computing device 108, or both, can also be implanted within the patient 104. In this way, the electrodes 102, the waveform generator 106, and the computing device 108 can define an implantable pulse generator.

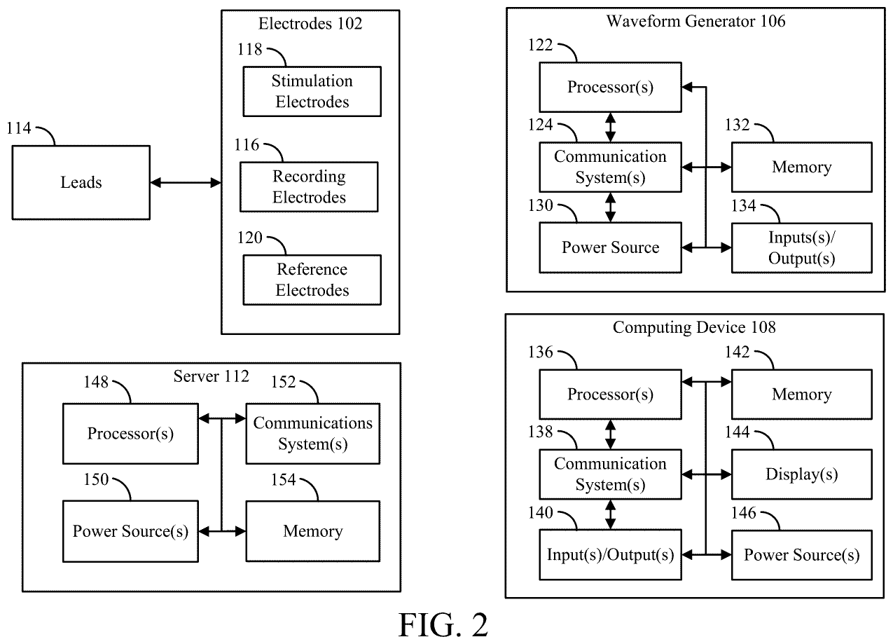

FIG. 2 shows a schematic illustration of specific block diagrams of the electrodes 102, the waveform generator 106, the computing device 108, and the server 112. The electrodes 102 can stimulation electrodes 116, recoding electrodes 118, and reference electrodes 120. In some cases, there can be 8, 16, 20, etc., electrodes 102 (e.g., contacts), with two of the electrodes 102 being stimulation electrodes 118, two of the electrodes being reference electrodes 120 (e.g., each of which can be electrically connected to ground), and the remaining electrodes can be recording electrodes 116. In some cases, the neurostimulation system 100 can include one or more leads 114 that can define the electrodes 102 (e.g., two leads). In some cases, each lead (and the corresponding electrodes) can be a commercially available lead for SCS, such as, for example, an Octrode™ lead.

The waveform generator 106 can include a processor(s) 122, communication system(s) 124, power source(s) 130, memory 132, and input(s)/output(s) 134. The processor 122 can be any suitable hardware processor or combination of processors, such as a central processing unit (“CPU”), a graphics processing unit (“GPU”), etc., which can execute a program (e.g., retrieved from memory 132) that can include the processes described below. The communication system 124 of the waveform generator 106 can include any suitable hardware, firmware, and/or software for communicating with other components of the neuromodulation system 100 (e.g., the computing device 108, server 112, etc.). For example, communications system 124 can include one or more transceivers, one or more communication chips and/or chip sets, etc. In a more particular example, communications system 124 can include hardware, firmware and/or software that can be used to establish a coaxial connection, a fiber optic connection, an Ethernet connection, a USB connection, a Wi-Fi connection, a Bluetooth connection, a cellular connection, etc. The power source 130 of the waveform generator 106 can embody many different forms. For example, the power source 130 can be a hardwired connection (e.g., a Universal Serial Bus connection), or it can be an electrical storage device (e.g., a battery). The power source 130 can supply power to all components within the waveform generator 106 (or other components, such as the computing device 108). In some specific cases, including when the waveform generator 106 is configured to be implanted in a patient, the power source 130 can be a non-rechargeable battery or a rechargeable battery. The memory 132 can include any suitable volatile memory, non-volatile memory, storage, or any suitable combination thereof. For example, the memory 132 can include random-access memory (“RAM”), static random-access memory (“SRAM”), read-only memory (“ROM”), electrically erasable programmable read-only memory (“EEPROM”), one or more flash drives, one or more hard disks, one or more solid state drives, one or more optical drives, etc. In some embodiments, the memory 132 can have encoded thereon a computer program for controlling operation of the processor 122. The input(s)/output(s) 134 of the waveform generator 106 can be indicators, sensors, actuated buttons, etc. For example, the inputs(s)/output(s) 134 can include light emitting diodes (“LEDs”), which can provide indication that the waveform generator 106 is powered on, and can indicate if the waveform generator 106 is supplying electrical stimulation to the electrode(s) 102. Additionally or alternatively, sensors or LEDs can indicate the magnitude, duration, or both of the current/voltage supplied to the electrodes 102 (e.g., the stimulation electrodes), via the waveform generator 106.

The computing device 108 can include processor(s) 136, communication system(s) 138, input(s)/output(s) 140, memory 142, display(s) 144, and power source(s) 146. The components described above with respect to the waveform generator 106 apply to the components of the computing device 108. In other words, components with the same name can have the same description and thus can be implanted in a similar manner. The display(s) 144 can be implemented in different ways. For example, the display 144 can be part of the computing device 108 (e.g., within the same housing), or separate from the computing device 108. In some embodiments, the display 144 can present a graphical user interface. In some embodiments, the display 144 can be implemented using any suitable display devices, such as a monitor, a touchscreen, a television, etc. In some embodiments, the input(s)/output(s) 140 of the computing device 108 can include indicators, sensors, actuatable buttons, a keyboard, a mouse, a graphical user interface, a touch-screen display, etc., so as to interact with images presented on the display 144 (e.g., the graphical user interface).

The server 112 can include processor(s) 148, power source(s) 150, communication system(s) 152, and memory 154, each of which can be implemented in a similar manner as the other like components above (e.g., with respect to the waveform generator 106).

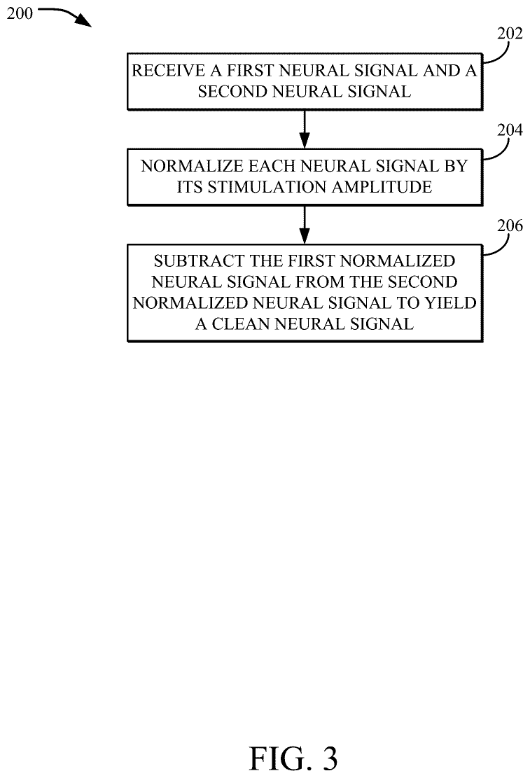

FIG. 3 shows a flowchart of a process 200 of cleaning neural signals (e.g., removing artifacts, and specifically stimulation artifacts from neural signals). The process 200 can be implemented using the neuromodulation system 100, and more specifically, can be implemented using one or more computing devices of the neuromodulation system 100 (e.g., one or more processors of the waveform generator 106, one or more processors 136 of the computing device 108, etc.).

At 202, the process 200 can include one or more computing devices, receiving a first neural signal and a second neural signal. In some cases, this can include the one or more computing devices causing a waveform generator to electrically excite a plurality of stimulation electrodes with a first stimulation amplitude, and, in response, receiving the first neural signal from a plurality of recording electrodes. This can also include the one or more computing devices causing the waveform generator to electrically excite the plurality of stimulation electrodes with a second stimulation amplitude (e.g., different form the first stimulation amplitude), and, in response, receiving the second neural signal from the plurality of recording electrodes. In some embodiments, the polarity of the first neural signal and the second neural signal is the same (e.g., the polarity applied to the stimulation electrodes is the same for both the first neural signal and the second neural signal). In some embodiments, electrical stimulation can occur with the same or different pairs of electrodes (e.g., a different plurality of electrodes). For example, the first neural signal can be acquired by stimulation a first plurality of the stimulation electrodes (e.g., two), while the second neural signal can be acquired by stimulating a second plurality of the stimulation electrodes (e.g., a different two). The same process can be implemented using the recording electrodes (e.g., since ECAPs propagate bidirectionally along the spinal cord from the site of stimulation). These features can be applied to the other processes herein, as applicable, such as process 250, 300, 350, 400, etc.

At 204, the process 200 can include the one or more computing devices normalizing each neural signal by its stimulation amplitude. For example, this can include the one or more computing devices normalizing the first neural signal by the first stimulation amplitude to yield a first normalized neural signal, and normalizing the second neural signal by the second stimulation amplitude to yield a second normalized neural signal. In some cases, the second stimulation amplitude can be greater than the first stimulation amplitude. In some cases, the first stimulation amplitude can be about (i.e., deviating by less than 30% from) 1 mA.

In some cases, although each stimulation amplitude of each stimulation waveform can be different, other properties of each stimulation waveform can be the same (or substantially the same, i.e., differing by less than 30 percent from). For example, the stimulation waveform can have the same shape (e.g. rectangular), can have the same width (e.g., pulse width), etc.

At 206, the process can include the one or more computing devices subtracting the first normalized neural signal from the second normalized neural signal to remove stimulation artifacts and yield a clean neural signal.

In some embodiments, the process 200 can include the one or more computing devices removing an artifact (e.g., a stimulation artifact) from a neural signal (e.g., a raw neural signal, based on the stimulation amplitude of the stimulations signal (or waveform) used to cause acquisition of the neural signal. As described below, the true or accurate components of neural signals vary non-linearly as stimulation amplitudes change, whereas stimulation artifacts vary linearly as stimulation amplitudes change. Accordingly, since the relationship between stimulation amplitude and amplitude magnitude is linear, the stimulation artifacts can be reliably removed without requiring the use of another neural signal (e.g., a template neural signal, such as the neural signal acquired at 1 mA of stimulation amplitude) and may be more computationally efficient (i.e., faster) than the subtracting neural signals, such as during a real-time application. For example, the one or more computing devices can receive a plurality of neural signals (e.g., raw neural signals), with each neural signal being acquired at a different stimulation amplitude. Each raw neural signal can be cleaned of the stimulation artifact, such as using the process in blocks 202-206. Then, the one or more computing devices can subtract the clean neural signal from the raw neural signal to yield a stimulation artifact (e.g., a value) for a given stimulation amplitude. Then, the one or more computing devices can determine the linear relationship between the stimulation artifacts and stimulation amplitudes (e.g., by plotting each pair of stimulation artifact at its stimulation amplitude and finding the best linear fit for the collective data). In this way, the one or more computing devices can remove the stimulation artifact from a subsequent neural signal by inputting the stimulation amplitude used to acquire the subsequent neural signal into the linear fit to yield a stimulation artifact and subtract the subsequent neural signal by the stimulation artifact to yield a clean neural signal.

In some embodiments, all the neural signals utilized in the process 200 can be derived, acquired, etc., from a single patient (i.e., the patient that is being electrically stimulated). In this way, treatments (e.g., stimulation waveforms, amplitudes, timing, etc.) can be tailored to the specific patient, since the anatomy, injury (e.g., type of injury and injury site, etc.), physiology, electrode locations, electrode placement, type of waveform generator, type of electrodes, number of electrodes, etc., of patients differ widely between each other, which can result in different neural signals. Accordingly, a patient specific treatment paradigm can be realized using all (or primarily all) the neural signals derived from the specific patient, which may yield better treatment results.

In some embodiments, when the polarity of the first neural signal is opposite to the polarity of the second neural signal, rather than the one or more computing devices subtracting the neural signals together, the first and second neural signals can be added together (e.g., summed) to effectively cancel out stimulation artifacts. However, as described below, such a process may be more prone to errors than the subtraction method.

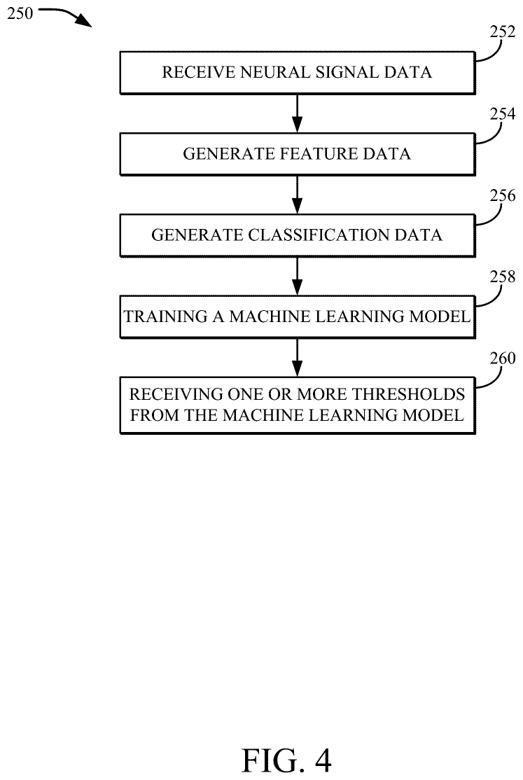

FIG. 4 shows a flowchart of a process 250 of training a machine learning model (e.g., to classify a neural signal as an ECAP, a non-ECAP, or an outlier, to output one or more thresholds for each feature). The process 250 can be implemented using the neuromodulation system 100, and more specifically, can be implemented using one or more computing devices of the neuromodulation system 100 (e.g., one or more processors of the waveform generator 106, one or more processors 136 of the computing device 108, etc.).

At 252, the process 250 can include one or more computing devices receiving neural signal data. The neural signal data can include a plurality of neural signals received in response to spinal cord stimulation. In some cases, each neural signal of a plurality of neural signals can be the average neural signal across multiple pairs of electrodes (e.g., the neural signal can be the average of a plurality of raw neural signals, where each neural signal is acquired from different pairs of electrodes). In some cases, the plurality of neural signals can be derived from one patient or multiple patients. The plurality of neural signals being derived from a single patient can advantageously result in a patient specific trained machine learning model, and accordingly result in patient specific determined thresholds (e.g., optimized thresholds). In some cases, each neural signal of the plurality of neural signals can be received at different stimulation amplitudes (e.g., of the spinal cord).

In some embodiments, all the neural signals utilized in the process 250 can be derived, acquired, etc., from a single patient (i.e., the patient that is being electrically stimulated). In this way, the classification (e.g., the trained machine learning model) can be patient tailored, which can better differentiate features and identify an ECAP from a single patient, rather than from a cohort of patients, since different patients can have widely different anatomies, injuries (e.g., type of injury and injury site, etc.), physiologies, electrode locations, electrode placements, types of waveform generator, type of electrodes, number of electrodes, etc., which can result in different neural signals. Accordingly, a patient specific treatment paradigm, and in this case, a patient specific machine learning model can be realized using all (or primarily all) the neural signals derived from the specific patient.

Each neural signal can be free of artifacts (e.g., stimulation artifacts). For example, each neural signal can cleaned of artifacts by normalizing the neural signal by its stimulation amplitude (e.g., the stimulation amplitude required to receive the neural signal), and can be subtracted by a normalized neural signal (e.g., a template normalized neural signal, that has been previously determined to be an ECAP and has been acquired at a stimulation amplitude, such as at about 1 mA) to yield a clean, artifact free, neural signal (e.g., clean of stimulation artifacts). This cleaning of neural signals of stimulation artifacts can be implemented in a similar manner to the process 200 described previously. Alternatively, each neural signal can be cleaned of artifacts by fitting a curve to each neural signal and subtracting the neural signal by the curve to yield a clean neural signal. In some cases, the curve can be an exponential curve (e.g., a single exponential function, a double exponential function, etc.) a polynomial curve (e.g., a second order polynomial function), a linear exponential function, etc. In some cases, the neural signal can be fit with the curve within a time window that includes a portion outside of a time window where ECAPs are present. For example, such as time window can be about 0.375 ms to 8 ms after a stimulation waveform has been applied to stimulation electrodes.

At 254, the process 250 can include the one or more computing devices generating feature data. In some cases, the one or more computing devices can generate the feature data by extracting features from the neural signal sin the neural signal data. In some cases, this can include the one or more computing devices performing a dimensionality reduction on the neural signal data to generate the feature data (e.g., including one or more features in the neural signal data). In some specific cases, the one or more computing devices can generate feature data by using principal component analysis (“PCA”), where the feature data includes one or more principal components (e.g., that can be stored in the memory of the one or more computing devices). Accordingly, generating feature data can be implemented (e.g., using the one or more computing devices) by an unsupervised learning method (e.g., since PCA is an unsupervised learning method).

At 256, the process 250 can include the one or more computing devices generating classification data, based on the feature data. In some cases, the classification data can include pairs of matched classification and neural signal data (or feature data). In some cases, the one or more computing devices can generate the classification data by clustering the data into different groups. For example, the one or more computing devices can use k-means clustering to cluster the data into different groups. Accordingly, generating classification data can be implemented (e.g., using the one or more computing devices) by using an unsupervised learning method.

At 258, the process 250 can include the one or more computing devices training a machine learning model using training data (e.g., using the classification data, the feature data, and the neural signal data). In some cases, the training data can be pairs of matched classification data and neural signal data (or feature data). In some cases, the machine learning model can be a k-nearest neighbors (“KNN”) classifier.

At 260, the process 250 can include the one or more computing devices can receive one or more thresholds from the machine learning model. For example, the one or more computing devices can assess the accuracy of the machine learning model (e.g., classifier) to determine how each feature (of the feature data) classifies a given neural signal as an ECAP, a non-ECAP, or an outlier, including determining a threshold (e.g., an optimal threshold, a predefined threshold, etc.) to use in classifying the given neural signal as an ECAP, a non-ECAP, or an outlier (e.g., the threshold differentiating between at least two classes of the three). In some cases, the threshold can be an optimal threshold for differentiating between two or more classes of signals (e.g., an ECAP, a non-ECAP, or an outlier). In some cases, a feature can be the area under the curve (“AUC”) of a neural signal) and the threshold corresponding to the AUC can differentiate between an ECAP and a non-ECAP. In this case, the area under the curve can be determined within a time window of between about 0.375 ms to about 2.375 ms, where time=0 is the time when a stimulation waveform is applied to the stimulation electrodes (e.g., the time window can be after the stimulation has been completed). In some cases, the AUC does not include the entire area of one or more positive peaks of the neural signal, but can include the entire area of a negative peak. In some cases, a feature can be the first principal component and the threshold corresponding to the first principal component can differentiate between an ECAP and an outlier. In some configurations, assessing the accuracy of the machine learning model can include using a receiver operating characteristic (“ROC”) curve.

In some embodiments, once the one or more thresholds have been received, the one or more thresholds can be stored in the memory of the one or more computing devices. In this way, an implantable device (e.g., an implantable pulse generator) can utilize optimal threshold for ECAP determinations during treatment (e.g., real time treatment). In some cases, such as when all (or substantially) all the neural signals used to train the machine learning model are derived from a single patient, the trained machine learning model (e.g., the block 258) can be a patient specific and accordingly the one or more thresholds received at the block 260 can each be patient specific.

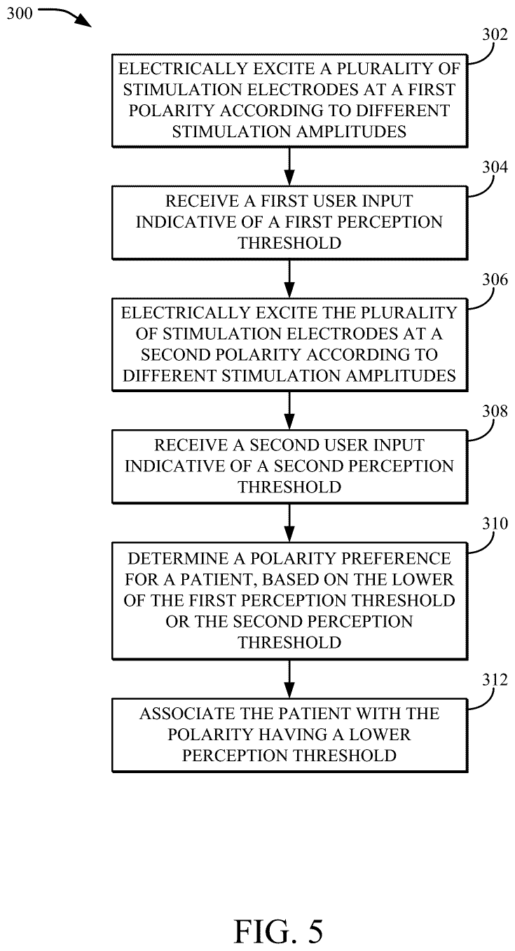

FIG. 5 shows a flowchart of a process 300 of determining a polarity preference for a patient. The process 300 can be implemented using the neuromodulation system 100, and more specifically, can be implemented using one or more computing devices of the neuromodulation system 100 (e.g., one or more processors of the waveform generator 106, one or more processors 136 of the computing device 108, etc.).

At 302, the process 300 can include the one or more computing devices electrically exciting a plurality of stimulation electrodes at a first polarity at different stimulation amplitudes. In some cases, a first electrode can be the positive electrode (e.g., the anode, during simulation) and a second electrode can be the negative electrode (e.g., the cathode, during stimulation. In other cases, the first electrode can be the negative electrode and the second electrode can be the positive electrode. In some cases, the stimulation waveform applied to the plurality of stimulation electrodes can dictate the first polarity. In some cases, the different stimulation amplitudes can increase step vise, such as beginning at 0 mA, and increasing by 0.5 mA until reaching 10 mA. These different stimulation amplitudes can be define a simulation amplitude window or a stimulation amplitude range.

At 304, the process 300 can include the one or more computing devices receiving a first user input (from the patient) indicative of a first perception threshold. For example, as the stimulation amplitude increases, there becomes a point when the user feels or perceives the stimulation. Once the patient feels the stimulation (e.g., after a few stimulation amplitudes delivered where the patient does not feel the stimulation), the user activates the user input device (e.g., a button, a touch screen, etc.) to deliver the first user input to the one or more computing devices. When the first user input has been received, the last stimulation amplitude delivered to the patient is set as the first perception threshold. In some cases, the first perception threshold for the first polarity of the stimulation electrodes can be further refined by implementing blocks 302 and 304 (a number of times, such as two, three, etc.) with a smaller stimulation amplitude window or stimulation amplitude range and with smaller step sizes of stimulation amplitude. In this way, the first perception threshold of the first polarity of stimulation electrodes can be further refined to yield a more accurate first perception threshold (e.g., in alignment with the patient's true perception threshold). In some cases, the process to repeat the blocks 302-304 is ended when the difference between perception thresholds (e.g., a current first perception threshold and a previous first perception threshold) is below a value. Further, the first stimulation amplitude used in other blocks of the process 300 (e.g., the block 310) can be the last stimulation determined for the first polarity.

At 306, the process 300 can include the one or more computing devices electrically exciting the plurality of stimulation electrodes at a second polarity at different stimulation amplitudes. The second polarity can be the opposite of the first polarity (e.g., with the polarity of the first electrode being reversed and with the polarity of the second electrode being reversed). The block 306 can proceed in a similar manner as the block 302, but with the stimulation amplitudes being reversed, if needed (e.g., negative amplitude in block 306 versus a positive amplitude in block 302, or vice versa).

At 308, the process 300 can include the one or more computing devices receiving a second user input (from the patient) indicative of a second perception threshold. This block 308 can be similar to the block 304, except with the different polarity applied to the plurality of stimulation electrodes. Similarly to blocks 302-304, the blocks 306-308 can be implemented a number of times to further refine the second perception threshold.

At 310, the process 300 can include the one or more computing devices determining a polarity preference for the patient, based on the lower of the first perception threshold and the second perception threshold. In some cases, this can include the one or more computing devices determining that the first perception threshold is below the second perception threshold, or vice versa. If the first perception threshold is below the second perception threshold, then the one or more computing devices determine that the patient has a polarity preference where stimulation electrodes be at a first polarity (e.g., when stimulating the spinal cord). If, however, the second perception threshold is below the first perception threshold, then the one or more computing devices determine that the patient has a polarity preference where stimulation electrodes be at a second polarity (e.g., when stimulating the spinal cord).

At 312, the process 300 can include the one or more computing device associating the patient with the polarity having the lower perception threshold. This can include the or more computing devices only stimulating the patient according to the polarity having the lower perception threshold. This can also include generating a report or otherwise notifying a user, such as a practitioner, that the patient has the polarity preference. As described below, electrically stimulating a patient with a polarity preference can advantageously result in lower stimulation amplitudes that otherwise elicit the same response. The lower current requirements can be healthier for patients and can be particularly advantageous in implantable systems where limiting current usage of batteries is important.

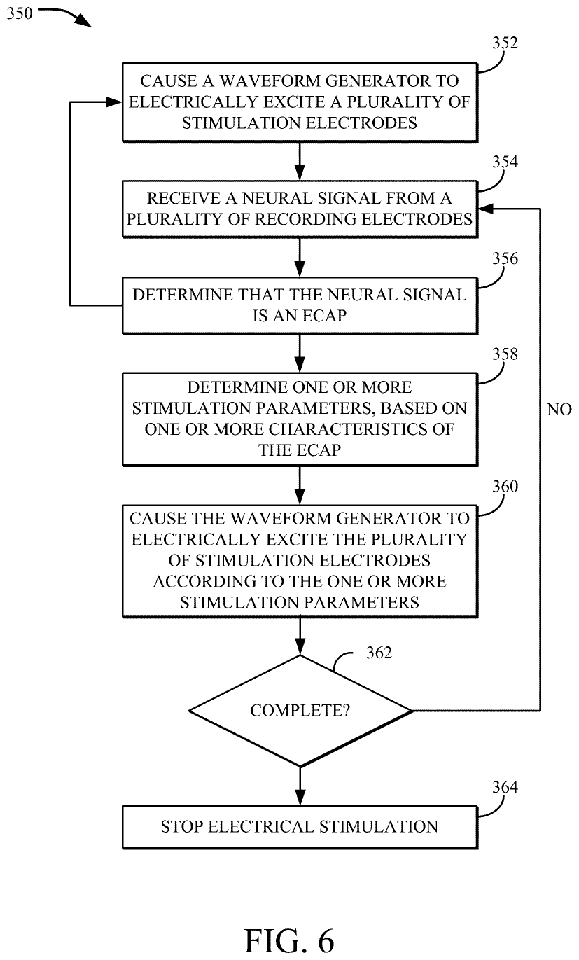

FIG. 6 shows a flowchart of a process 350 of treating one or more conditions of a patient (e.g., using electrical stimulation, such as of the spinal cord). The one or more conditions can include pain, incontinence (e.g., urinary incontinence, fecal incontinence such as bowel dysfunction, etc.), a lost function (e.g., the treatment restoring a lost function, particularly those with a spinal cord injury), a spinal cord injury (e.g., a complete spinal cord injury or an incomplete spinal cord injury), an abnormal blood pressure (e.g., a systolic blood pressure (“SBP”), such as a high SBP), abnormal muscle spasms, sexual distress (e.g., female sexual distress), etc. In some cases, the process 350 can improve the synergy of multiple groups of muscles. The process 350 can be implemented using the neuromodulation system 100, and more specifically, can be implemented using one or more computing devices of the neuromodulation system 100 (e.g., one or more processors of the waveform generator 106, one or more processors 136 of the computing device 108, etc.).

At 352, the process 350 can include the one or more computing devices causing a waveform generator to electrically excite a plurality of stimulation electrodes, which can be implemented in a similar way to the other similar blocks of the processes above. In some cases, electrically exciting the stimulation electrodes can be according to patient's polarity preference.

At 354, the process 350 can include the one or more computing devices receiving a neural signal from a plurality of recording electrodes. Again, the block 354 can be implemented in a similar manner to the other similar blocks of the processes above. In some cases, the one or more computing devices can clean the neural signal according to the other processes described herein (e.g., subtracting the neural signal by a normalized ECAP, subtracting the neural signal by a curve, etc.). In some cases, the neural signal can be acquired after a time delay (e.g., a banking period) after stimulating the stimulation electrodes at the block 352 (e.g., where stimulating occurs at time=0). In some cases, the time delay can be about 0.375 milliseconds. In some cases, the neural signal can be received or acquired during a window of about 0.375 milliseconds to about 2.375 milliseconds after the beginning of stimulating the stimulation electrodes (e.g., where t=0 occurs at the leading edge of the stimulation waveform).

At 356, the process 350 can include the one or more computing devices determining that the neural signal (e.g., received, acquired, etc., at the block 354) is an ECAP. In some cases, this can include the one or more computing device determining that the neural signal is not a non-ECAP, not an outlier, and is an ECAP. More specifically, the one or more computing devices can determine (1) that the neural signal is an ECAP and is not an non-ECAP, and (2) that the neural signal is an ECAP and is not an outlier. In some cases, the one or more computing devices determine that the neural signal is an ECAP based on both determinations (1) and (2). In some cases, the one or more computing devices can determine one or more features of the neural signal (e.g., one or more principal components, the peak to peak amplitude (“P2P”) of the neural signal, the area under the curve (“AUC”) of the neural signal, the average slope of the neural signal, the average voltage of the neural signal, the latency of a negative peak of the neural signal, etc.), and can determine that the neural signal is an ECAP based on the one or more features of the neural signal. In some configurations, if the one or more computing devices determine that that the neural signal is a non-ECAP or an outlier, the process 350 can proceed back to the block 352. In this case, one or more stimulation parameters (e.g., amplitude, pulse width, etc.) can be adjusted and then used by the waveform generator to electrically excite the plurality of stimulation electrodes (or other stimulation electrodes).

In some cases, the one or more computing devices only determine that the neural signal is an ECAP (and proceed to the block 358) after the one or more computing devices determine that the neural signal is an ECAP for each condition (of one or more conditions, such as (1) and (2) above).

At 358, the process 350 can include the one or more computing devices determining one or more stimulation parameters, based on the one or more characteristics of the ECAP (e.g., the neural signal determined to be an ECAP). In some cases, after properly identifying that the neural signal is an ECAP, the neural signal determined to be an ECAP can be used as a control signal for closed loop feedback (e.g., the ECAP being the feedback signal). For example, the P2P of the ECAP (e.g., between N1 and P2) can be determined by the one or more computing devices and can be used to determine the one or stimulation parameters (e.g., amplitude, pulse width, etc.). As one non-limiting example, one or more control algorithms can be used to decide whether or not to turn the energy in the stimulation up or down based on the P2P of the amplitude. For example, a proportional-integral-derivative (PID) controller can be used or reinforcement learning can be used.

At 360, the process 350 can include the one or more computing devices causing the waveform generator to electrically excite the plurality of stimulation electrodes (or other stimulation electrodes), according to the one or more stimulation parameters.

At 362, the process 350 can include the one or more computing devices determining that the stimulation has been complete. In some cases, stimulation (and these underling processes) can proceed continuously (e.g., for chronic pain), intermittently (e.g., while a person is performing an activity), etc. If at the block 362, the one or more computing devices determine that stimulation is complete, the process 350 can proceed to the block 364 where the one or more computing devices cause the waveform generator to cease electrically exciting the stimulation electrodes (e.g., for a period of time). If, however, at the block 362, the one or more computing devices do not determine that stimulation is complete, the process 350 can proceed back to the block 354 to receive a subsequent neural signal from a plurality of electrodes (e.g., either the plurality of electrodes or a different plurality of electrodes).

In some embodiments, all the neural signals utilized in the process 350 can be derived, acquired, etc., from a single patient (i.e., the patient that is being electrically stimulated). In this way, stimulation, classification, treatment can be patient tailored. Patient tailoring of classification and stimulation can provide better treatment outcomes (e.g., can be more accurate) as described above. This also applies to the process 400.

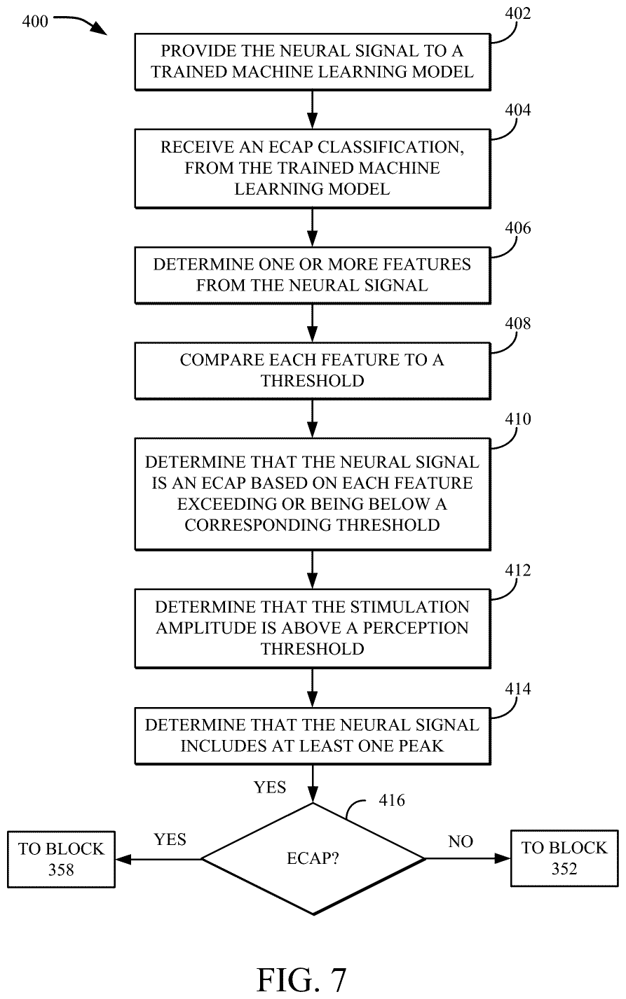

FIG. 7 shows a flowchart of a process 400 of determining whether a neural signal is an ECAP, which can be a specific implementation of the block 358 of the process 350. Accordingly, the process 400 can be implemented using the neuromodulation system 100, and more specifically, can be implemented using one or more computing devices of the neuromodulation system 100 (e.g., one or more processors of the waveform generator 106, one or more processors 136 of the computing device 108, etc.).

At 402, the process 400 can include the one or more computing devices providing a neural signal to a trained machine learning machine (e.g., a patient specific trained machine learning model). In some cases, the neural signal has been cleaned of artifacts (e.g., stimulation artifacts). In some cases, the output of the trained machine learning can be a classification of the neural signal as an ECAP, a non-ECAP, or an outlier. In some embodiments, providing the neural signal to the trained learning machine can include the one or more computing devices extracting the one or more features from the neural signal and classifying the neural signal based on the one or more extracted features.

At 404, the process 400 can include the one or more computing devices determining an ECAP classification for the neural signal, form the trained machine learning model. In this case, the one or more computing devices can determine that the neural signal is an ECAP, based on receiving the ECAP classification for the neural signal from the trained machine learning model. As is apparent, the trained learning machine can be trained by using the processes herein (e.g., the process 250). As described above, the blocks 404-406 can be a first condition (e.g., of multiple conditions before determining that the neural signal is an ECAP).

At 406, the process 400 can include the one or more computing devices determining one or more features from the neural signal. The one or more features can include one or more principal components (e.g., PC1, PC2, PC3, PC4, PC5, etc.), one or more parameters (e.g., P2P, AUC, the average slope of the neural signal, the average voltage of the neural signal, or the latency of a negative peak (i.e., N1) of the neural signal).

At 408, the process 400 can include the one or more computing devices comparing each feature to a corresponding threshold. Each threshold that corresponds to a specific feature can be an optimized threshold and can be determined using the processes described herein (e.g., the process 250). In some cases, each threshold can be determined from a patient specific machine learning model (e.g., the machine learning model having been trained on data received from the same patient being electrically stimulated at the process 350). In some cases, the computing device can determine whether each feature is below or above a corresponding feature threshold.

At 410, the process 400 can include the one or more computing devices determining that the neural signal is an ECAP, based on each feature (e.g., one or more features) exceeding or being below a corresponding threshold. Similarly, to the blocks 402-404, the blocks 406-410 can represent a second condition collectively, or the analysis of each feature can represent its own condition.

At 412, the process 400 can include the one or more computing devices determining that the stimulation amplitude is above a perception threshold (e.g., for the patient). In some cases, ECAPs are only consistently seen when stimulation amplitudes exceed the perception threshold. So, this can be an easy, non-computationally intense, way to ensure that neural signals collected are actually ECAPs. In some configurations, the perception threshold can be the perception threshold for a preferred polarity of the patient and where stimulation has occurred according to the preferred polarity (e.g., at the block 352). In this block, the one or more computing devices can determine that the neural signal is an ECAP, based on the stimulation amplitude (e.g., at the block 352) being above the perception threshold. Again, similarly to the other blocks, the block 412 can be a condition.

At 414, the process 400 can include the one or more computing devices determining that the neural signal has at least one peak. In some cases, this can include the one or more computing devices determining that the neural signal has at least two peaks, or three peaks. More specifically, this can include the one or more computing devices determining that the neural signal has three peaks, with one peak being a positive peak (i.e., P1), a second being a negative peak (i.e., N1), and the third being a positive peak (i.e., P2), with the negative peak being positioned between the positive peaks (e.g., in the time domain). In general, ECAPs have characteristics peaks, and thus ECAPs can be determined based on the presence of at least one characteristic peak. For example, if the neural signal includes no peaks, it is likely that the neural signal is not an ECAP (which typically includes three peaks). If however, the neural signal includes at least one peak (or other peaks), the one or more computing devices can determine that the neural signal is an ECAP (e.g., in essence scrubbing neural signals that are extremely unlikely to be ECAPs). Similarly to the other blocks, the block 414 can be a condition.

At 416, the process 400 can include the one or more computing devices determining whether the neural signal is an ECAP. The one or more computing devices can determine that the neural signal is an ECAP, based on each of the above one or more conditions having been passed. For example, the one or more conditions can be any number of the previously described conditions (e.g., machine learning classification, exceeding an optimal threshold, etc.). In some cases, the one or more computing devices determine that the neural signal is an ECAP only after all conditions have been passed. In some configurations, however, some analyses can be computationally intensive (e.g., finding peaks at the block 414), and thus those conditions are not used (e.g., where computational recourses are limited, such as in an implantable system), or are saved for later analyses only after non-computationally intensive conditions are passed. Accordingly, the one or more computing devices can evaluate each condition based on computational intensity of the evaluation (e.g., each condition can be serially arranged by computational complexity, where the lowest computational complexity condition is evaluated first, the second lowest computational complexity condition is evaluated second, and so on). In this way, possibly computationally limited resources are better utilized. In other cases, the conditions can include only the thresholds (to optimize computational resources). In some cases, the one or more computing devices can determine that the neural signal is not an ECAP after having failed one or more of the conditions. In this case, the process 400 can proceed to the block 352 to cause the waveform generator to electrically excite the stimulation electrodes (or other plurality of stimulation electrodes). Alternatively, if at the block 416, the one or more computing devices determine that the neural signal is an ECAP, the process 400 can proceed to the block 358 of the process 350.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

Example 1 provides feature extraction and prediction of spinal cord stimulation evoked compound action potentials in humans.

### Introduction

Evoked compound action potentials (“ECAPs”) during spinal cord stimulation (SCS) may be useful in the treatment of chronic pain as a control signal for closed-loop neuromodulation. However, considerable inter-individual variability in evoked responses requires robust methods in order to realize effective, personalized pain management. These methods include artifact removal, feature extraction, classification, and prediction.

### Methods

ECAPs from eight participants with chronic pain undergoing an externalized trial with two percutaneous leads were recorded. The two most caudal electrodes were used for stimulation and the remaining electrodes were used for recording. Artifact-cleaned waveforms were clustered using principal component analysis (“PCA”) and classified using a K-Nearest Neighbors (“KNN”) classifier as non-ECAPs, ECAPs, or outliers (i.e., artifacts) to determine how well different features, including area under the curve (“AUC”) and peak-to-peak amplitude (“P2P”), discriminate between waveform classes. Finally, generalized linear mixed effects models (GLMEs) were used to predict evoked response features and the probability of observing artifacts or ECAPs following individual stimulation pulses for different stimulation amplitudes, pulse widths, and polarities.

### Results

AUC was better at discriminating between ECAPs and non-ECAPs than P2P (d′=2.44 vs d′=48 2.27) while most features were good at discriminating between ECAPs and artifacts (d′>1.5). 49 The application of an optimal AUC threshold was then used to analyze individual ECAPs at 50 different stimulation amplitudes, pulse widths, and polarities. Interestingly, ECAPs could be evoked using ˜1.25 mA less current when using participant-specific, preferred stimulation polarities. Conversely, N1 latency consistently correlated with the location of the cathode.

### Conclusion

An automated analysis pipeline for individual ECAPs during SCS was developed. AUC was better than the widely used P2P for characterizing evoked responses. Furthermore, our modeling results provide a method for predicting how various stimulation parameters affect SCS responses in individual participants. The study was registered on ClinicalTrials.gov (#NCT04938245).

Epidural spinal cord stimulation (“SCS”) is an established treatment for chronic pain typically delivered with periodic pulses and often programmed ad-hoc or optimized through iterative programming based on patient feedback. When SCS is applied in the epidural space of the spine, epidural spinal recordings (“ESR”) collected from non-stimulation contacts on the implanted leads can contain various useful biological signals such as evoked compound action potentials (“ECAPs”) from the spinal cord dorsal column, evoked electromyography (“EMG”) from nearby contracting muscles, cardiac signals, respiratory signals, etc. Moreover, the ECAP component of ESR can be used in closed loop applications of SCS.

ECAP signals typically appear in a time window (ECAP window) within 2 ms in the ESR immediately following stimulation, and ECAPs have a characteristic triphasic morphology commonly referred to as P1, N1 and P2. Recently, the ECAP component in ESR has been used in closed-loop SCS therapy where stimulation amplitude was adjusted based on ECAP amplitude. In these systems the ECAP amplitude was estimated by using peak-to-peak (“P2P”) amplitude of the negative and positive peaks in the ECAP window. Typically, P2 is estimated as the maximum, and N1 is the minimum value within the ECAP window without doing true peak detection (e.g. finding the local maxima).

While P2P amplitude describes a major component of ECAPs, ECAPs are complex waveforms composed of both varying amplitude and dispersion (i.e., width) evoked from dorsal column fibers. Others have begun to explore the utility of the width of the negative peaks to assess the morphology of the ECAP signals. To capture this ECAP morphology, a later study quantified ECAPs using area-under-curve (“AUC”) within the ECAP window. While AUC and P2P are correlated, AUC captures more than just amplitude. Because it is debatable which single feature is the best option to quantify the ECAP component in ESR, it is necessary to comprehensively investigate the feature space to determine which feature is best for future algorithmic or closed-loop applications.

P2P amplitude is also prone to contamination of stimulation artifacts because the capacitive artifact is often recovering through the entire ECAP window. This phenomenon is more severe for recording electrodes that are proximal to the stimulation site. A previous study has also shown that differences between the reference strategies could distort the recorded evoked responses. In addition, stimulation artifacts can dramatically change with body position caused by relative movements between the implanted leads and spinal cord. The artifact effects then could lead to movement dependent distortion of the recordings in the ECAP window and further contaminate P2P measurements that are used to quantify ECAP signals. As a result, relying on P2P values to represent ECAP amplitude in current commercial systems may lead to artifact-driven misinterpretations, potentially compromising the accuracy of evoked response assessments. Therefore, it is essential to implement supplementary analyses that first verify the presence of genuine ECAP signals before attempting any quantification, ensuring the accuracy of subsequent measurements.

Past research has mainly focused on the presence of ECAP signal alone and its P2P amplitude as a quantitative approach to measure local neural activity to stimulation and determine effective stimulation. However, further research about quantification and feature analysis of the characteristic waveforms in ECAP window include but are not limited to: amplitude, latency, average slope, average voltage, and AUC. These specific features may provide better insight on the relationship and underlying dynamics between different stimulation parameters and their effects on neural activity.

Another challenge is in identifying the best stimulation parameters for individual participants, which remains an open area of research. Research has shown that different stimulation polarities on the same electrodes could result in different response outcomes due to the underlying anatomy and current flow differences between different stimulation polarities. It is clinically meaningful and important to investigate how evoked responses in ESR, such as ECAP features, might be used to indicate and quantify various response outcomes between different stimulation polarities on the same group of contacts. Lower stimulation amplitude would be preferred if possible for improving battery life and facilitating signal processing by reducing stimulation artifacts. In addition, if a lower stimulation amplitude can induce the same effective evoked responses, then a higher stimulation amplitude should be avoided to eliminate challenges associated with overstimulation.

In this study, the differences between various waveform features were investigated—such as P2P and AUC—and how well these features can discriminate between non-ECAPs, ECAPs and outliers (i.e., artifacts). It was hypothesized that the application of advanced machine learning techniques could accurately classify ESR waveforms as non-ECAPs, ECAPs, and outliers. Furthermore, it was hypothesized that individual stimulation parameters (i.e., amplitude, pulse width, and polarity) could predict the probability of eliciting ECAPs despite significant inter-participant variability. Specifically, a variety of machine learning techniques were employed including Principal Component Analysis (“PCA”), k-means, K-Nearest Neighbors (“KNN”) classification, and ROC analysis to determine which feature was best at discriminating between waveform classes while minimizing human biases. Optimal feature thresholds were used, determined by the ROC analysis, with Logistic Generalized Linear Mixed Effects models (“GLMEs”) to determine how stimulation parameters predict the probability of observing ECAPs and outliers. How these features relate to stimulation parameters was explored, including stimulation amplitude, pulse width, and polarity using Linear Mixed Effects Models (“LMEs”). Like many other studies it was observed that a large amount of inter-participant variability existed in SCS ESRs, but the statistical power of LMEs and GLMEs was leveraged to account for this variability, allowing for the identification of common trends across participants that can be applied to large data sets.

### Methods

### General

The methods outlined in our previous work (i.e., “Methods and System for Recording Human Physiological Signals from Implantable Leads During Spinal Cord Stimulation,” Ramadan et al., 2023) were closed followed All analyses were carried out in Matlab 2024a (Mathworks Inc., Natick Massachusetts) using custom written code. All participants consented to this study under approval by the University of Minnesota's IRB (#STUDY00013100), which complies with the Declaration of Helsinki. The study was also posted on ClinicalTrials.gov (#NCT04938245).

### Participants

Fifteen participants were recruited with chronic pain implanted with two percutaneous Octrode™ leads (model #3086, Abbott Neuromodulation, Plano, Tx) with externalized leads. However, there was considerable variability in quality of the signals recorded. Therefore, 30 sessions were selected from 8 participants with consistent stimulation patterns, no more than 2 bad electrodes, and recordings were carried out using an Atlas Neuralynx (Neuralynx, Bozeman, MT) (Table 1, below). Two sessions from one participant were later removed because electrode alignment could not be confirmed in these sessions due to observing very small ECAPS on a limited number of electrodes even when stimulating at 10.0 mA with a 150 μs pulse width. Participants completed up to 6 stimulation sessions over 3 visits.

TABLE 1
Participant Demographics & Inclusion/Exclusion

| Participant | | | Included | Recording | Stimulation | | Stimulator | Exclusion |
|---|---|---|---|---|---|---|---|---|
| ID | Age | Sex | (Yes/No) | Equipment | Equipment | Grounding | Level | Reason/Comments |
| eCAPS01 | M | 56 | Yes | Atlas | Atlas Stim | Ground | Thoracic | Channel 2 was bad |
| | | | | | Headbox | patch* | | |
| eCAPS02 | M | 53 | Yes | Atlas | Atlas Stim | Onlead | Thoracic | Ground channel 16 |
| | | | | | Headbox | | | |
| eCAPS03 | M | 62 | No | Atlas | Atlas Stim | Onlead | Thoracic | Technical Issues |
| | | | | | Headbox/IPG | | | |
| eCAPS04 | F | 66 | No | Atlas | Cerestim/IPG | Onlead | Thoracic | Technical Issues |
| eCAPS05 | M | 62 | No | Atlas | TDT | Ground | Thoracic | Amplitude range from |
| | | | | | | patch | | perception to |
| | | | | | | | | intolerable was limited |
| | | | | | | | | to < 1 mA. |
| eCAPS06 | M | 49 | Yes | Atlas | TDT | Ground | Thoracic | |
| | | | | | | patch | | |
| eCAPS07 | F | 68 | No | Atlas | TDT | Ground | Cervical | Stimulation amplitude |
| | | | | | | patch | | ranges limited and |
| | | | | | | | | required 0.1 mA step |
| | | | | | | | | sizes. |
| eCAPS08 | F | 57 | No | TDT | TDT | Ground | Cervical | No Atlas recordings & |
| | | | | | | patch | | stimulation amplitude |
| | | | | | | | | ranges limited and |
| | | | | | | | | required 0.2 mA step |
| | | | | | | | | sizes. |
| eCAPS09 | M | 37 | Yes | Atlas | TDT | Ground | Thoracic | |
| | | | | | | patch | | |
| eCAPS10 | F | 72 | Yes | Atlas | TDT | Ground | Thoracic | Channel 10 was |
| | | | | | | patch | | reference since channel |
| | | | | | | | | 9 was bad |
| eCAPS11 | M | 50 | No | Atlas | TDT | Ground | Thoracic | Left lead was partially |
| | | | | | | patch | | pulled out and |
| | | | | | | | | stimulation on contacts |
| | | | | | | | | 7/8 was not effective. |
| eCAPS12 | F | 71 | Yes | Atlas | TDT | Ground | Thoracic | Channel 10 is reference |
| | | | | | | patch | | since channel 9 & 11 |
| | | | | | | | | were bad |
| eCAPS13 | M | 66 | Yes | Atlas | TDT | Ground | Thoracic | Channel 12 was bad. |
| | | | | | | patch | | 150 μs sessions |
| | | | | | | | | excluded due to |
| | | | | | | | | ECAPS being too small |
| | | | | | | | | for post hoc alignment. |
| eCAPS14 | F | 69 | No | Atlas | TDT | Ground | Thoracic | Technical issues? No |
| | | | | | | patch | | perception even at 10 |
| | | | | | | | | mA on 150 μs rapid |
| | | | | | | | | perception increased for |
| | | | | | | | | 350 μs. |
| eCAPS15 | F | 25 | Yes | Atlas | TDT | Ground | Cervical | |
| | | | | | | patch | | |

*A disposable Natus EMG Ground Plate Electrode was placed on the skin of the lower back.

### Experimental Design

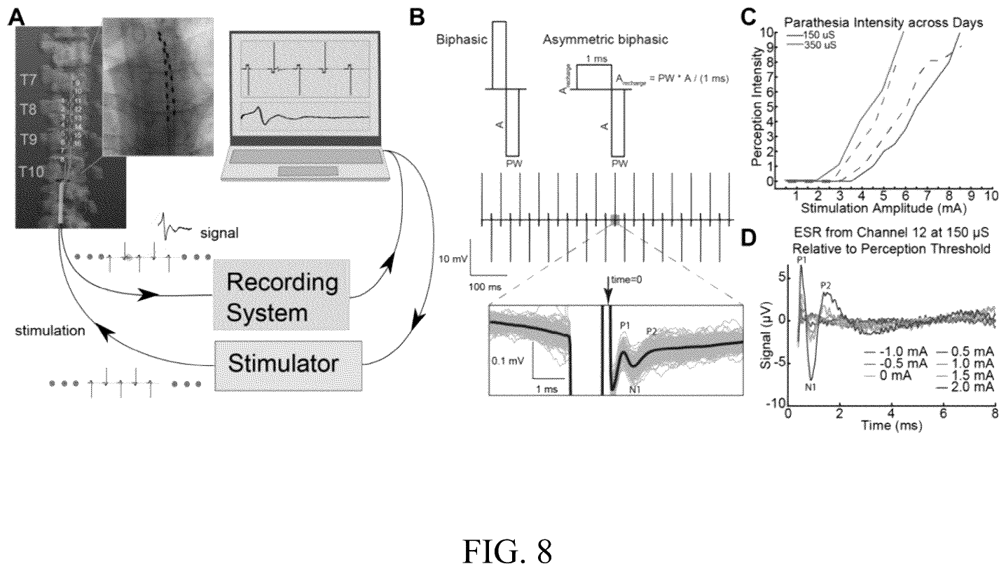

Externalized leads were directly connected to an Atlas Neuralynx system (Neuralynx, 163 Bozeman, MT). Raw data was recorded at 32 kHz with a 24 bit resolution with an input range of 164 132 mV (see FIG. 8A). Either an Atlas Stim Headbox (2 participants) or a TDT system (6 participants) delivered tonic stimulation on the two most caudal electrodes (electrodes 7 & 8). Channel 9 or 10, 166 on the contralateral electrode, was used as the reference electrode since it was usually the most distal recording electrode from the stimulation sites. The Neuralynx ground was connected to a grounding patch on the back in all but 1 session in 1 participant who had on-lead ground on channel 1 instead. The remaining electrodes served as recording electrodes.

In each session asymmetric, charged-balanced, biphasic tonic stimulation was delivered at pulse widths of 150 μs or 350 μs for 10 seconds at 38 Hz (see FIG. 8B); a 1 ms initial charging phase was used followed by a discharge phase of the desired pulse width. Individual sessions were conducted with a single stimulation pulse width. Stimulation intensity was stepwise from 0.5 mA in 0.5 mA increments until the participants reached their maximum stimulation intensity tolerance, stimulation amplitude reached motor threshold, or the hardware maximum stimulation amplitude of 10.0 mA was reached. Stimulation amplitude was constant during the 10 second stimulation period. To test the effects of stimulation polarity, alternated the anode and cathode was alternated on every stimulation pulse.

Participants were asked to not move during stimulation; participants were allowed to lay down or sit up during stimulation, whichever was most comfortable. Position of the participant could vary across sessions and days. During the stimulation period, participants were asked to rate the paresthesia intensity of the stimulation with 0 being no sensation and 10 being the maximum sensation imaginable (see FIG. 8C). Typically, clear ECAPS were only observed for stimulation amplitudes of 1 mA or greater than perception (see FIG. 8D). In total, 33.1% of waveforms were collected below the perception threshold, and 46.5% of waveforms were collected at less than 1 mA above perception threshold.

FIG. 8 provides an experimental overview. FIG. 8A shows a stimulation setup where participants were implanted with two percutaneous Octrode™ leads near the thoracic region of the spinal cord. Externalized leads were connected to an Atlas NeuraLynx 192 recording system and stimulation was delivered using an Atlas Stim Headbox or a TDT system. FIG. 8B shows stimulation trains, where they were delivered for 10 seconds at 38 Hz using asymmetric, charged-balanced, biphasic stimulation pulses with alternating polarity on contacts 7 & 8. FIG. 8C shows a graph where paresthesia intensity was measured during stimulation. Paresthesia intensity varied slightly from day to day (different lines) within a participant though paresthesia intensity consistently increased more rapidly for 350 μS pulse widths (orange) than 150 μS 197 pulse widths (blue). FIG. 8D shows a graph of the average ESR signals from a single channel in the same participant as in FIG. 8C for stimulation amplitudes relative to perception threshold showing that an ECAP was not readily observed at perception threshold or detected at stimulation amplitudes just above perception threshold. *Approximate P1, N1, P2 times are labeled in FIGS. 8B & D.

### Signal Processing

Raw Neuralynx data were imported into Matlab for analysis using custom written code. A median filter with a window length of 100 ms was used to detrend the data. Individual stimulation pulse times were obtained from the stimulation artifacts on channel 6, and stimulation evoked responses for each channel were extracted from the data from 5 ms before the stimulation pulse to 8 ms after the stimulation pulse to confirm alignment.

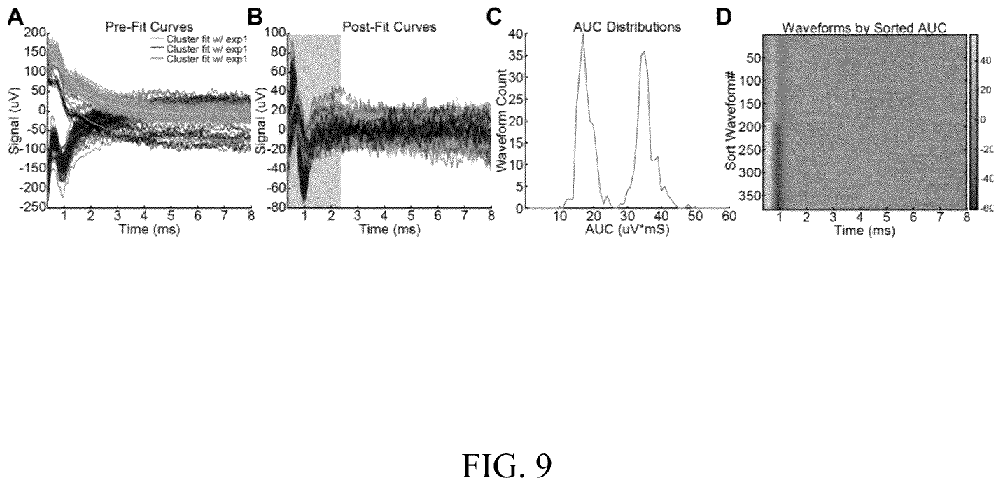

To extract the evoked response, stimulation artifacts were fit from 0.375 ms to 8 ms after stimulation. First, stimulation artifacts were grouped using PCA and k-means clustering (see FIG. 9A); the number of clusters was determined by silhouette width. Typically there were 2 clusters representing the different polarities but sometimes short time-scale signal drift and other artifacts (e.g., heartbeat) caused there to be more than 2 clusters. Second, the mean artifact waveform was fit with various exponentials (exp1, exp2, or exp2 fit to the artifact derivative) or polynomial (poly1, poly2, or poly4) functions using Matlab's fit function. A complexity cost was applied to reduce the likelihood of over-fitting with more complicated fitting functions favoring exponential fits (1, 1.5, and 2) over polynomial fits (2, 4, and 6), respectively. The best fit was determined as the minimum mean squared error (“MSE”) multiplied by the complexity cost. Third, the cluster-averaged artifact was subtracted from each evoked artifact and then subtracted out the mean. This resulted in “artifact-cleaned” waveforms which were used in all subsequent analyses (see FIG. 9B).

Multiple fitting functions were used as stimulation artifacts varied across electrodes, signals were analyzed on recording electrodes proximal to the stimulation sites (e.g., channel 6) which were extensively contaminated by artifacts; normally, these electrodes would simply be removed from analysis. Overall, stimulus artifacts were fit and removed with some form of exponential function in 99.2% of waveforms.

### Feature Extraction

Features were extracted from a 2.0 ms ECAP window from 0.375 ms to 2.375 ms as most of the ECAP waveform was contained in this time window across all participants. Several features were then extracted from the individual waveforms within this ECAP window: Area Under the Curve (“AUC”), Peak to Peak (“P2P”) amplitude, N1 amplitude, number of peaks and valleys, average voltage, and average slope (see FIGS. 9C & 9D). AUC was calculated as the mean corrected AUC in the time window. P2P was calculated as the absolute maximum minus the absolute minimum value within the time window. Peaks and valleys were detected using findpeaks in Matlab. To determine good peak detection parameters, a parameter sweep was conducted on averaged evoked waveforms during stimulation with 150 μs pulse widths. Specifically, artifact cleaned-waveforms 1 mA below perception and 2 mA above perception were used, where it was expected to have non-ECAPs and ECAPs waveforms, respectively. Peak detection parameters were chosen with a false positive rate of approximately 0.5 and a power of 0.80. A minimum peak amplitude of 7.5 V, minimum time between peaks of 0.25 ms, and a minimum peak width of 0.15625 ms identified peaks (or valleys) with an approximate false positive rate of 0.29 and statistical power of 0.79 was found. Average voltage was calculated as the average voltage value within the ECAP window. Average slope was the average instantaneous slope within the ECAP window. Note, for most analyses the magnitude (∥) of the average voltage and average slope was used.

FIG. 9 shows waveform preprocessing and feature extraction from a 6 mA stimulation. FIG. 9A shows a graph of Post-stimulation (0.375 to 8.0 ms) waveforms being clustered (different colors) in PCA space using k-means clustering and artifacts were fit for each cluster using an exponential fit. Typically, 1 cluster per stimulation polarity was found, but because of movement, respiratory, or heartbeat artifacts there were sometimes more clusters (black lines). FIG. 9B) shows artifact-cleaned waveforms for each cluster revealing ECAPs. Gray window 253 (0.375 to 2.375 ms) was the ECAP window used in feature extraction. FIG. 9C shows a graph of the distribution of AUC values from artifact-cleaned waveforms in FIG. 9B showing bimodal distribution presumably related to stimulation polarity. FIG. 9D shows waveforms sorted by AUC again showing bimodal distribution in AUC as well as potentially a small latency shift in the evoked responses presumably related to stimulation polarity.

### Electrode Alignment

Perioperative X-rays of electrode positions were obtained from clinical records. X-ray images were processed using a custom made GUI and the Matlab Image Processing Toolbox to extract relative electrode positions. First, labeled electrode positions were manually labeled as some electrodes appeared to be touching or overlapping, and the image contrast varied across participants. Second, X-ray images were then automatically thresholded and turned into a black and white image. Third, Matlab's bwlabel then automatically identified electrodes as clusters of white pixels. Fourth, automated and manually labeled electrodes were compared, and when electrodes were overlapping or touching, automated electrode positions were fixed. Fifth, the final electrode position was determined from the center-of-mass of white labeled pixels.

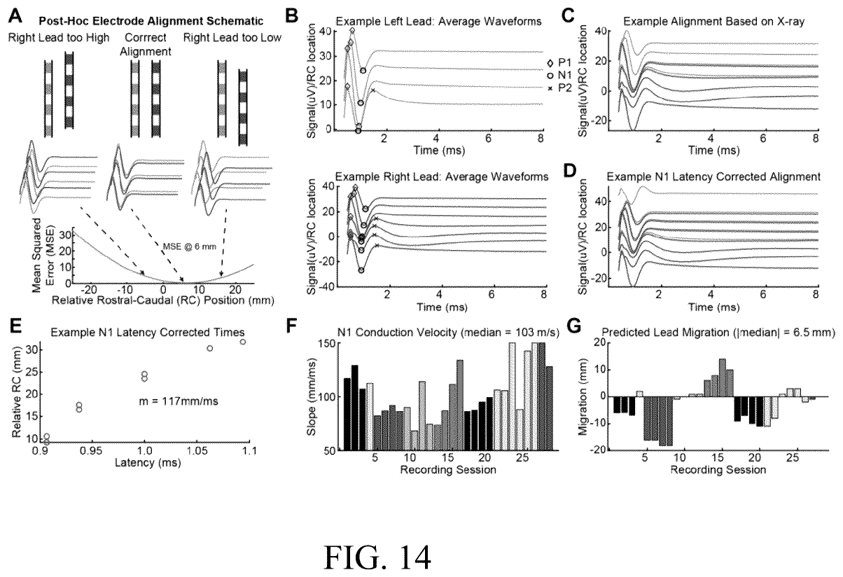

Electrode alignment was initially based on the peri-operative X-rays, but waveform shapes and N1 latencies suggested lead migration had occurred in most participants (see FIG. 14). To estimate the amount of lead migration, N1 latencies were used on averaged waveforms, averaged across polarities as this negated artifacts on channels close to the stimulation sites even though averaging across polarities distorts the signal. Averages were calculated only from stimulation amplitudes that elicited evident ECAPS. A simple linear regression of N1 latencies versus relative rostral-caudal position of the leads was used to calculate lead offset. The relative position of the leads were programmatically adjusted, the N1 latency data was regressed, and then the MSE was calculated. The alignment of the left and right leads was determined as the relative position of the leads that minimized the MSE of this linear regression. Median estimated lead migration was 6.5 mm with a range of 0 to 18 mm.

### ECAPs Classifier

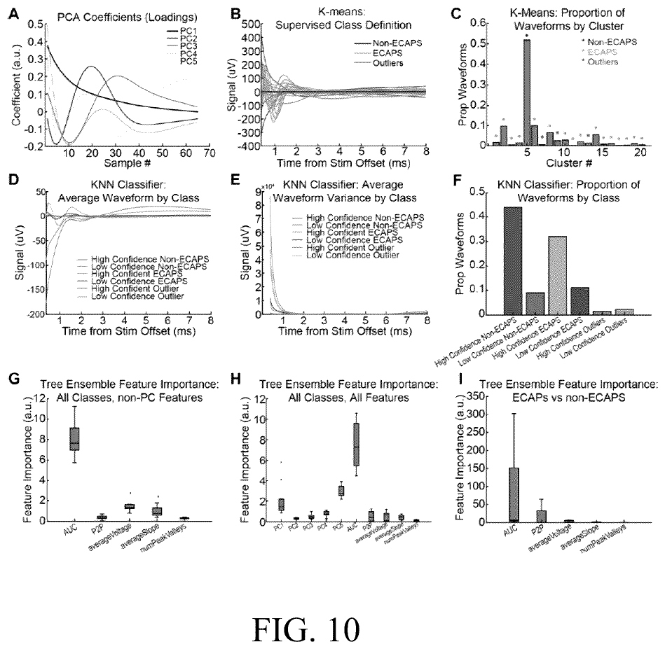

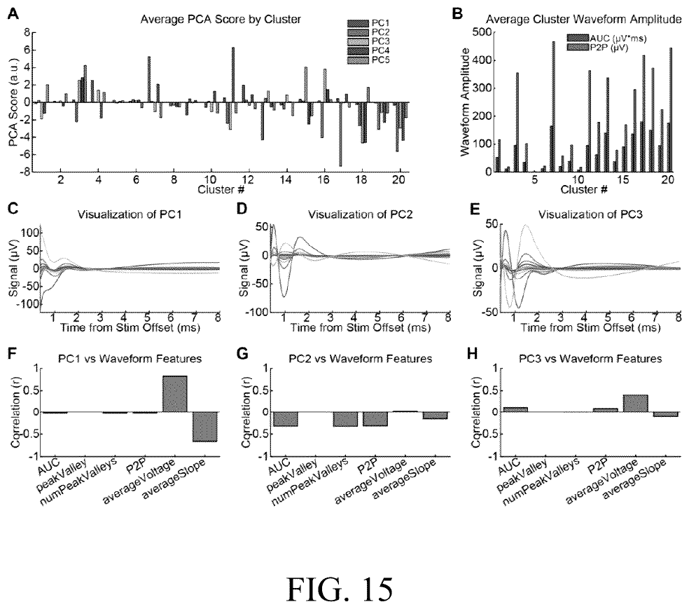

A combination of PCA (See FIGS. 10A, 15), k-means clustering, and a KNN classifier was used to identify the best feature for discriminating between non-ECAPs, ECAPs and outliers. First, PCA was applied to all of the waveforms to reduce the dimensionality of the data. The first 5 principal components (“PC”) were used, which explained 97.6% of the variance in the data. Then extreme outliers (20 MAD (“Median Absolute Deviation”)) in PCA space were removed.

Second, a nested k-fold cross-validation was performed using every 11th waveform in each outer loop. Within each outer loop, an unsupervised algorithm k-means was used to cluster the data. Initially, k-means was used to split the data into more manageable sizes using k=4. Then k-means was applied a second time using k from 4 to 12, where k was determined by silhouette width. For each cluster, outliers (greater than 10 MAD) in PCA space were removed. Then clusters as representing non-ECAPs, ECAPs, or outliers based on the average waveform within each cluster (see FIGS. 10B, 15B) were labeled (supervised).

Clusters with average waveforms without any detectable peaks or valleys and P2P less than 15 μV were determined to represent non-ECAPs. Clusters with average waveforms without any detectable peaks or valleys with P2P greater than 15 μV were determined to represent outliers. Clusters with average waveforms with at least one detectable peak or valley and with P2P greater than 15 μV were determined to represent ECAPs. 15 μV was used as this was twice our minimum peak amplitude determined by the peak detection parameter sweep. Any ambiguous clusters were removed from subsequent classification. Next, a KNN classifier was built based on these labeled data. A 5-fold cross-300 validation was used to determine the accuracy of the KNN classifier. Additionally, waveform class labels were considered to have high-confidence if the KNN classifier said the waveform likely belonged to a class with greater than 95% probability; otherwise, waveforms were labeled as low-confidence.

### ROC Analysis & Feature Importance

Feature importance, optimal thresholds, and feature discriminability were determined within each outer loop. Median values across outer loops were then reported and used for further analysis. To determine the importance of each feature, a series of classification tree ensemble models were built. Waveform class from the KNN classifier was predicted and classification tree ensemble models were then built (fitcensemble.m) based on different waveform features combinations. In Matlab predictorImportance was used to determine feature importance.

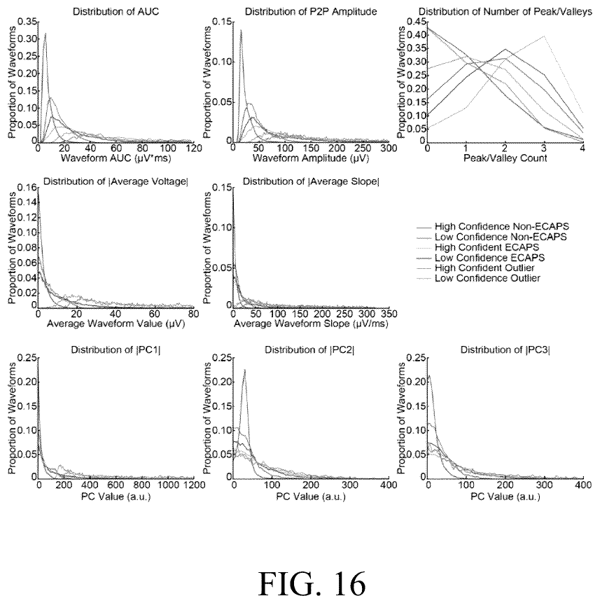

ROC analysis was used to determine optimal thresholds and to assess how well individual features discriminate between different waveform classes. Only waveforms that were classified were used, with high-confidence waveforms and their features for this analysis as low-confidence waveforms had intermediate feature distributions (See FIG. 16). ROC analysis was carried out independently for each feature between non-ECAPs and ECAPs as well as between ECAPs and outliers. The magnitude (∥) of the PC values, average slope, and average voltage was used for this analysis as values could be both positive and negative. Positive and negative values for these features were generally caused by artifact polarity. Youden's J-statistic was used to determine the optimal threshold, and d-prime (d′) was calculated from the area under the ROC curve to determine class-based feature discriminability.

### GLME Analyses

There was considerable intra-participant and inter-participant variability in evoked responses. Additionally, across participants there was variability in the stimulation amplitudes used and the number of sessions completed. Therefore, simple averages would be biased towards participants with the most data at different stimulation parameters. To account for this variability, LMEs (fitlme) and Logistic GLMEs (fitglme) were used to understand the relationship between waveform features, stimulation parameters, and recording distance. For Logistic GLMEs the data was fit using the canonical link function ‘logit’ and always calculated dispersion. For all waveform features, except N1 latency, the following formula in Wilkinson's notation was used: waveformFeature˜stimAmplitude+stimPulseWidth+stimPolarity+recordingDistance+(1|participant).

Since N1 latency is a function of recording distance and since lead migration was accounted for on a session-by-session basis, session was used as a random effect instead of participants. Additionally, there was a concern that larger amplitude waveforms may have N1 latencies that were more reliably detected so AUC was used as a predictor to correct for the effects of stimulation amplitude and stimulation polarity on evoked response amplitude. Therefore, the N1 latency formula was as follows: n1Latency˜stimAmplitude+stimPolarity+recordingDistance+AUC+(1|session).

Linear models were also built for each session using the same predictors to verify these results were not an artifact of lead migration correction across sessions.

### Results

### Building an ECAPs Classifier to Determine the Best Features for ECAP Detection

One goal was to determine the best waveform features for classifying waveforms as having non-ECAPs, ECAPS, or outliers. To reduce human biases, the PCA was calculated for all artifact-cleaned waveforms (n=1,855,304). Visual inspection of the PCA coefficients (see FIG. 10A) and PC sorted waveforms (See FIG. 15) suggested that the 1st PC correlated with the initial magnitude and slope of the artifact recovery in the evoked waveform, while the remaining PCs appeared to correlate with various peaks and valleys.

First, on each outer loop, unsupervised clustering was performed of the waveforms using k-means clustering and the first 5 PCs, which explained 97.6% of the variance in the data. The total number of clusters across outer loops varied from 18 to 32 (median 21). The average cluster waveforms appeared to represent different waveform classes (see FIG. 10B). Clusters 359 (supervised) were labeled as representing non-ECAPS, ECAPs, or outliers based on the cluster's averaged waveform's P2P and the presence or absence of peaks and valleys (See FIG. 10B/C). Typically, 1 very large cluster was labeled as representing non-ECAPs, numerous clusters were labeled as representing ECAPs with different amplitudes and latencies, and a few clusters were labeled as representing outliers from different stimulation polarities.

Second, a KNN classifier was built from these labeled cluster data. It was found that a KNN classifier could accurately classify waveforms with a median 5-fold cross-validation accuracy of 97.7% across outer loops. Importantly, the KNN classifier also produced posterior probabilities indicating how likely a waveform belonged to a particular class. The average of the high confidence (>95% posterior probability) waveforms appeared to match their expected waveforms, while low confidence (<95% posterior probability) waveforms appeared more like the average waveform between classes (see FIG. 10D). The variance of the waveform amplitudes also increased from non-ECAP to ECAP to outlier clusters, indicating the KNN classifier also captured some of the waveform variance too. On average, 59.7% of the waveforms were classified as high-confidence non-ECAPs, 9.0% of waveforms as low-confidence non-ECAPs, 19.3% of waveforms as high-confidence ECAPs, 8.2% of waveforms as low confidence ECAPs, 1.3% of waveforms as high-confidence outliers, and 1.7% of waveforms as low-confidence outliers (see FIG. 10F).

Third, tree ensemble models were used and applied to all the waveforms and their features to determine the best feature for predicting waveform class as determined by the KNN classifier. Importantly, AUC had the highest feature importance whether only non-PCA features was used (see FIG. 10G), all features were used (see FIG. 10H), or only non-PCA features were used to classify just non-ECAPs versus ECAPs (see FIG. 10I).

Fourth, these tree ensemble results were confirmed using ROC analysis. For the ROC analysis, the high-confidence waveforms were only analyzed (Table 2, below). AUC was the best (d′=2.44) feature for discriminating between non-ECAP and ECAP waveforms. Interestingly, PC1 was the best (d′=3.26) feature at discriminating between ECAP and outlier waveforms. However, all non-PCA features had d′ values greater than 1.6 suggesting that outliers were generally easy to classify, and average voltage and average slope had d′ values greater than 2.

TABLE 2
ROC Analysis of Classifier Results*†

| | Non-ECAPS vs ECAPS | | | ECAPS vs Outliers | | |
|---|---|---|---|---|---|---|
| | | Optimal | | | Optimal | |
| Feature | d' | Threshold | | d' | Threshold | |
| \|PC1\| | 1.03 | 41.9 | a.u. | 3.26 | 411.9 | a.u. |
| \|PC2\| | 0.98 | 51.6 | a.u. | 0.17 | 189.0 | a.u. |
| \|PC3\| | 1.77 | 46.0 | a.u. | 0.09 | 227.5 | a.u. |
| \|PC4\| | 1.75 | 36.6 | a.u. | 0.09 | 89.9 | a.u. |
| \|PC5\| | 2.28 | 29.0 | a.u. | 0.82 | 42.0 | a.u. |
| AUC | 2.44 | 18.9 | μV*ms | 1.64 | 80.0 | μV*ms |
| P2P | 2.27 | 49.0 | μV | 1.75 | 198.2 | μV |
| \|Average Voltage\| | 1.18 | 5.3 | μV | 2.14 | 26.5 | μV |
| \|Average Slope\| | 1.33 | 12.7 | μV/ms | 2.29 | 85.1 | μV/ms |

| | | | | |
|---|---|---|---|---|
| numPeak Valleys | 1.23 | 2 | 1.62 | 2 |

*obtained from high-confidence waveforms only
†median values across outer loops

In summary, these results indicate that a waveform classifier can be built with minimal human intervention using PCA, k-means clustering, and a KNN classifier. From these results it was found that AUC was the best single feature at discriminating between non-ECAPs and ECAPs while PC1 was the best feature for discriminating between ECAPs and outliers.

FIG. 10 shows the building of an ECAPs classifier using PCA, k-means clustering, and a KNN classifier. FIG. 10A shows that PCA was used to extract nonparametric features from all evoked waveforms, and then k-means clustering was used for unsupervised grouping of waveforms. FIG. 10B shows that from the cluster's averaged waveforms, clusters were then labeled as representing non-ECAPs (blue), ECAPs (green), outliers (red) based P2P and the presence or absence of detectable peaks and valleys. FIG. 10C shows ECAPS and outliers were represented by several clusters each, while there was only 1 cluster representing non-ECAPS. FIG. 10D shows that mean evoked waveforms based KNN classifier labeled waveforms further divided into high- and low-confidence waveforms. FIG. 10E shows the mean variance of evoked waveforms based on a KNN classifier labeled waveforms clustered further divided into high- and low-confidence waveforms. FIG. 10F shows the proportion of waveforms classified by the KNN classifier as non-ECAPs, ECAPs, and outliers further divided into high- and low-confidence waveforms. FIGS. 10G and I show relative feature importance according to tree ensemble models using waveform classes determined by the KNN classifier for FIG. 10G all non-PCA features with all classes, for FIG. 10H all features with all classes, and for FIG. 10I for all non-PC features for non-ECAP and ECAP classes only. *PCA FIG. 10A was applied to all waveforms, while k-means clustering FIG. 10B-C and KNN classification FIG. 10D-F were applied within each outer loop; FIGS. 10B-F show example data from a single outer loop. FIGS. 10G-I show distribution of feature importance across outer loops.

Applying Optimal Threshold from the ROC Analysis to Classify Evoked Responses

Because PCA analysis in real-time applications may not be feasible, the optimal thresholds determined in the ROC analysis were used to classify all the waveforms using the features with the best discriminating ability to classify each waveform as an outlier, ECAP, or non-ECAP. Since PC1 was the best feature for discriminating between ECAPs and outliers and was correlated with average voltage and average slope, the evoked waveforms were classified as outliers if the waveforms' average voltage was greater than 26.5 V OR the waveforms' average slope was greater than 85.1 V/ms. After identifying outliers, the remaining waveforms were classified using an AUC threshold of 18.9 V*ms to discriminate ECAPs from non-ECAPs responses. Using these simple thresholds, 59.0% of waveforms were identified as non-ECAPS, 30.1% of waveforms were identified as ECAPS, and 10.9% of waveforms were identified as outliers. In the next sections the relationships between stimulation parameters, recording location, waveform features, and waveform class using LMEs and GLMEs were explored.

### Factors Predicting Outlier Classification

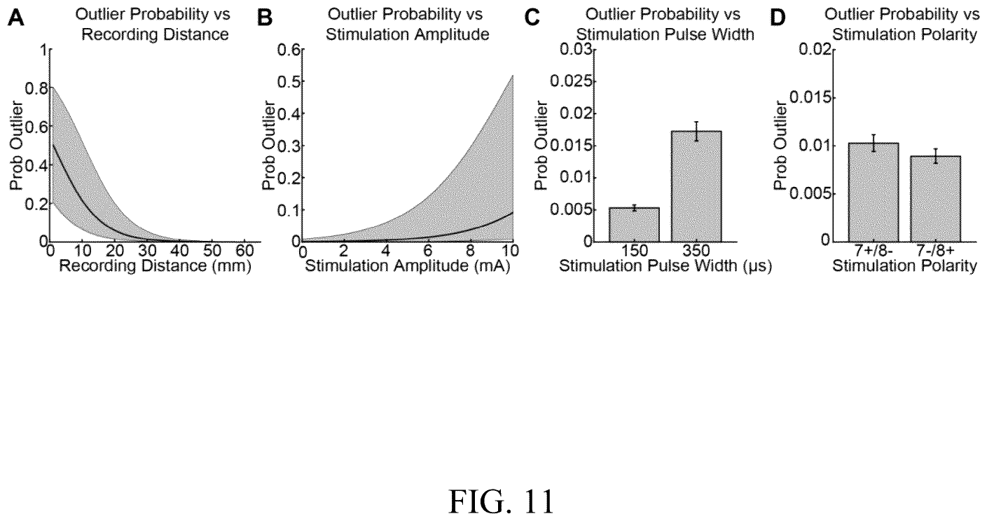

Characterizing and handling stimulation artifacts and stimulation effects remains a ubiquitous challenge in closed-loop neuromodulation. It is expected that stimulation artifacts will increase with stimulation amplitude and be worse for recording electrodes closer to the stimulation sites. To quantify these relationships, a logistic GLME was built to investigate the relationship between the probability of detecting outliers and these parameters (see FIG. 11). Recording distance was found to be a major predictor of outliers (OR=0.86 per mm) with the probability of detecting outliers considerably more probable for recording sites proximal to the stimulation location (See FIG. 11A). Moreover, stimulation amplitude was also a major predictor of outliers (OR=1.60 per mA) where outliers were more probable for higher stimulation amplitudes though the GLME predicted considerably more spread around the median at high amplitudes (see FIG. 11B). The GLME predicted that recording electrodes closer to the stimulation sites were impacted more by higher stimulation amplitudes compared to recording electrodes far from the stimulation site (data not shown). Stimulation pulse width (OR=1.006 per S) also had a modest impact on outlier probability (see FIG. 11C), while stimulation polarity (OR=0.93 per polarity) had little impact on outlier probability (see FIG. 11D).

These results indicate that removing electrodes close to the stimulation sites was necessary before conducting further analysis. At a 15 mm recording distance from, the GLME predicted that 11.7% of waveforms were outliers so recording channels were removed closer than 15 mm to the stimulation sites. Additionally, any channels in a session with >10% of its waveforms classified as an outlier were removed. Furthermore, recording channels below the stimulation sites or above the reference electrode were removed as it was found that these waveforms could also be distorted despite not being classified as outliers.

Overall, 43.6% of waveforms across all sessions were removed. Of the removed waveforms, 23.4% were outliers, 37.0% were ECAPs, and 39.6% were non-ECAPs. Of the remaining waveforms, 73.9% were non-ECAPs, 24.8% were ECAPs, and 1.2% were outliers. Before any further analysis, the 1.2% of waveforms that were classified as outliers were also removed.

FIG. 11 shows a logistic GLME model visualization of factors predicting outlier classification using optimal thresholds calculated from the ROC analysis. FIG. 11A shows a graph of the recording distance, FIG. 11B shows a graph of the stimulation amplitude, and FIG. 11C shows a graph of the stimulation pulse width, each of which were strong predictors of outlier classification while stimulation polarity FIG. 11D was not. All plots show median+/−IQR calculated across all other predicted values. The Logistic GLME model predicted that the large spread around the median in FIG. 11B was due to the increased susceptibility to artifacts at higher stimulation amplitudes on recording electrodes proximal to the stimulation sites.

### Factors Predicting Waveform Features

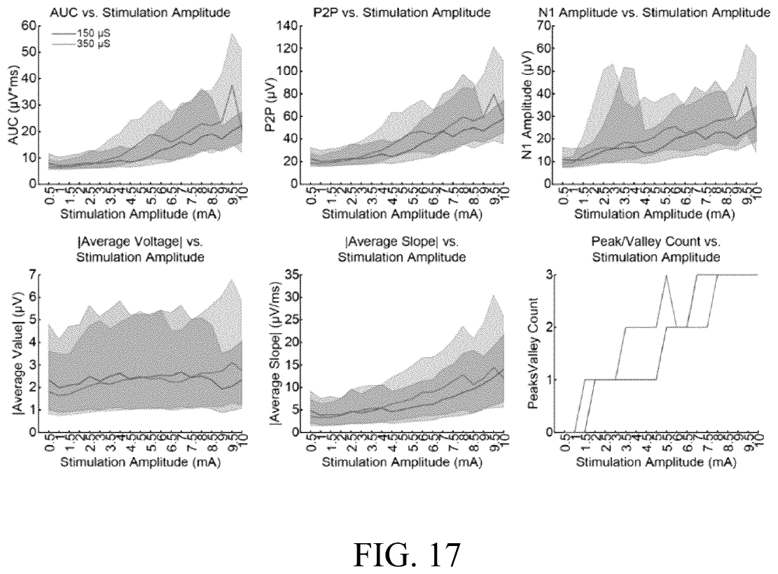

There was considerable inter-participant variability in ECAP waveforms (see FIG. 17) so it was investigated how different stimulation parameters and recording distance predicted waveform feature values using LMEs (see Table 3, below). Stimulation amplitude was a moderate to strong predictor of all features (ηₚ²'s 0.13 to 0.30). Stimulation pulse width had a small to moderate effect on all features (ηₚ²'s 0.28 to 0.12), while stimulation polarity had a very small effect on all features 467 (ηₚ²'s≤0.17). Recording distance had a small effect on all features (all ηₚ²'s˜0.5) suggesting that most features change similarly with recording distance beyond 15 mm. Interestingly, not all models performed equally well. The variance explained (R²) by each of the feature models ranged from 0.20 to 0.41. LMEs explained a good amount of variance for AUC, P2P, and N1 amplitude (all R²˜0.4) but did not explain as much variance for average voltage or average slope (R²'s˜0.2) possibly because these features are better predictors of artifacts.

TABLE 3
Beta Coefficients & Effect Sizes (ηₚ²) for LME Models for Waveform Features

| | Stimulation | Stimulation | Stimulation | Recording | |
|---|---|---|---|---|---|
| Waveform | Amplitude | Pulse Width | Polarity | Distance | |
| Feature | (per mA) | (per μs) | (per polarity) | (per mm) | R² |
| AUC | 3.13 | 0.038 | −1.43 | −0.33 | 0.41 |
| | (ηₚ² = 0.29) | (ηₚ² = 0.074) | (ηₚ² = 0.014) | (ηₚ² = 0.057) | |
| P2P | 7.72 | 0.084 | −2.74 | −0.81 | 0.41 |
| | (ηₚ² = 0.30) | (ηₚ² = 0.064) | (ηₚ² = 0.0091) | (ηₚ² = 0.058) | |
| N1 | 5.82 | 0.080 | −2.42 | −0.45 | 0.40 |
| Amplitude* | (ηₚ² = 0.25) | (ηₚ² = 0.12) | (ηₚ² = 0.017) | (ηₚ² = 0.040) | |
| \|Average | 0.25 | 0.0034 | −0.095 | −0.12 | 0.20 |
| Voltage\| | (ηₚ² = 0.30) | (ηₚ² = 0.065) | (ηₚ² = 0.0091) | (ηₚ² = 0.058) | |
| \|Average | 1.51 | 0.018 | −0.77 | −0.24 | 0.22 |
| Slope\| | (ηₚ² = 0.13) | (ηₚ² = 0.028) | (ηₚ² = 0.0068) | (ηₚ² = 0.049) | |
| Peaks/Valley | 1.27 | 1.002 | 0.93 | 0.99 | |
| Count* | | | | | |

*For waveforms with detectable N1 times
**Poisson GLME, exponential beta coefficients (rate ratio)

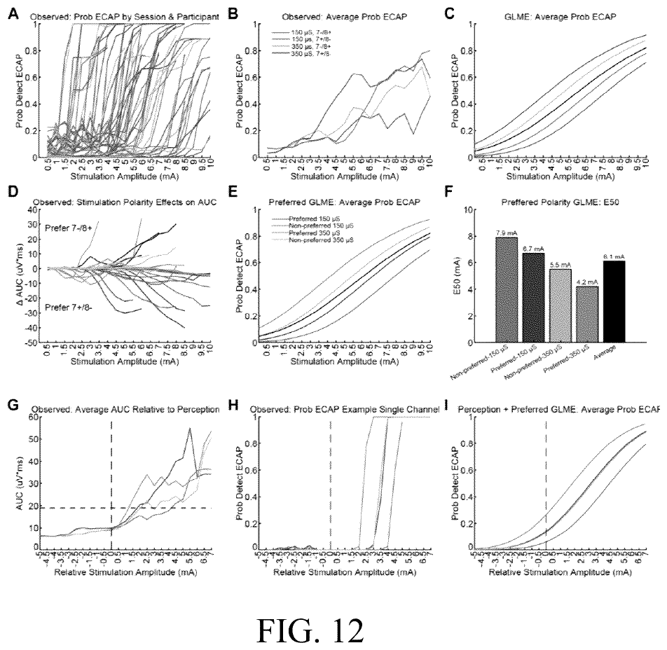

FIG. 12 shows factors predicting ECAP classification using optimal thresholds calculated from the ROC analysis. FIG. 12A shows a graph of the average probability of detecting an ECAP for individual sessions across multiple channels separated by stimulation polarity and pulse width shows a lot of inter-participant and intra-participant variability. FIG. 12B shows a graph of the average probability of detecting an ECAP across all participants and sessions separated by stimulation polarity and pulse width. Note there is unequal sampling across stimulation parameters. FIG. 12C shows a GLME model fit to the data in B, which shows that stimulation polarity and pulse width affect the probability of detecting an ECAP. FIG. 12A shows a graph of the difference in AUC across stimulation polarity for each session (different shades) indicating the effects of preferred stimulation polarity on ECAP AUC. Note sessions could have different stimulation pulse widths. FIG. 12E shows a graph of the Updated GLME model predictions to the data using preferred stimulation polarity. FIG. 12F shows a graph of E50 from the GLME model using preferred stimulation polarity indicating that preferred stimulation polarity needs ˜1.25 mA less current to evoke an ECAP. FIG. 12G shows a graph of the observed average AUC relative to perception threshold, which showed an abrupt increase at stimulation amplitudes above perception particularly for the preferred stimulation polarity. FIG. 12H shows a graph of the probability of detecting an ECAP relative to perception threshold from participant 9 on channel 3 from a single day. FIG. 12I shows a graph of the GLME model fit to the data aligned to the perception threshold across all participants.

### Factors Predicting ECAPs

As with other features, a lot of variability was observed in the probability of detecting an ECAP across sessions and participants (see FIG. 12A). However, there was a higher probability of detecting ECAPs for higher stimulation amplitudes and longer pulse widths across participants (see FIG. 12B). To better understand the relation between stimulation parameters and the probability of detecting an ECAP, a Logistic GLME was built (see FIG. 12C). The GLME found that the probability of detecting an ECAP increased with stimulation amplitude (OR=2.65 per mA) and stimulation pulse width (OR=1.01 per μS). Conversely, the probability of detecting an ECAP decreased slightly with recording distance (OR=0.93 per mm). Interestingly, the probability of detecting an ECAP was also substantially affected by stimulation polarity (OR=0.64 per polarity).

From the minimally processed data (see FIG. 17), it appeared that for different participants different stimulation polarities affected ECAP amplitude and the probability of observing an ECAP at certain stimulation amplitudes. Therefore, the AUC's were analyzed for each session as a function of stimulation amplitude and polarity. 18 sessions from 4 participants in which the stimulation polarity 7−/8+ evoked larger potentials than a stimulation polarity of 7+/8−, and 10 sessions from the other 4 participants showed larger evoked potentials for 7+/8− compared to 7−/8+ (see FIG. 12D). The absolute maximum AUC difference ranged from 2.6 μV*ms to 40.1 μV*ms suggesting a large variability across sessions and participants though the preference generally was similar within each participant. For some sessions there also appeared to be a plateau effect at higher amplitudes suggesting that ECAP amplitude grows at a similar rate for different stimulation polarities once ECAPs were reliably evoked.

A new Logistic GLME was built with stimulation polarity preference instead of the electrode polarity to better understand these polarity effects (see FIG. 12E). It was found that the probability of detecting an ECAP increased with stimulation amplitude (OR=2.74 per mA) and pulse width (OR=1.01 per μS) as well as with preferred polarity (OR=1.90 per polarity). The probability of detecting an ECAP decreased slightly with recording distance (OR=0.92 per mm). To better understand the effect of preferred polarity on the probability of detecting ECAPs, E50 was calculated from the GLME as the stimulation amplitude in which ECAPS were detected 50% of the time across participants and sessions. It was found that E50 decreased by 1.2 to 1.3 mA for the preferred polarity for both stimulation pulse widths (see FIG. 12F).

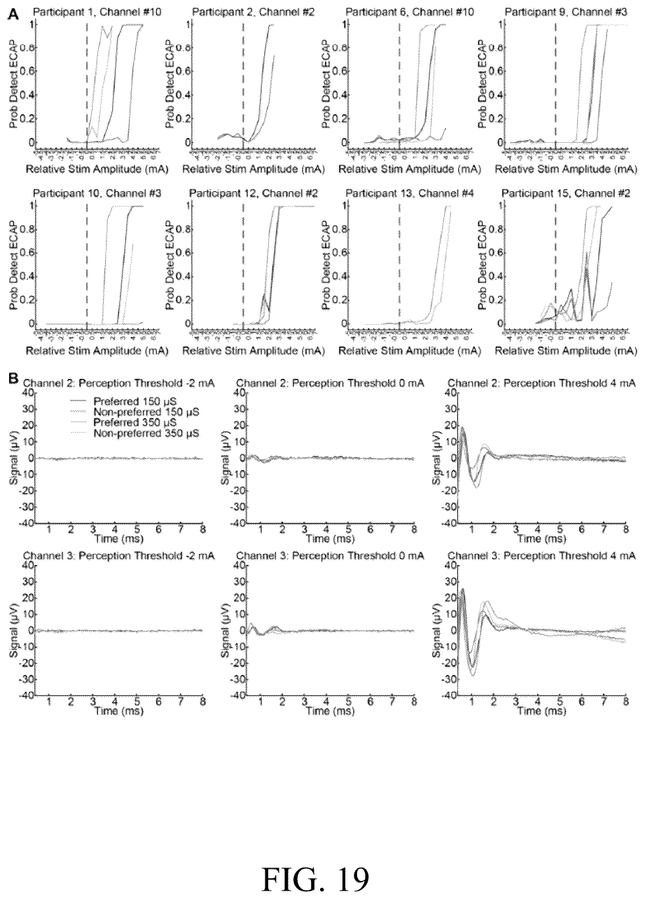

Lastly, the probability of detecting an ECAP relative to the perceptual threshold was analyzed. Based on the perceptual data, 46.8% of waveforms were expected to have ECAPs, but only 30.1% of waveforms were classified as having an ECAP. To try to explain this discrepancy, the data was realigned to the perception threshold. This realignment suggested AUC increased abruptly at stimulation amplitudes above perceptual threshold (see FIG. 12G), and that ECAPS were only detected above perception threshold in all participants (see FIG. 12H, see FIG. 19). Additionally, the probability of detecting an ECAP was more closely aligned to perception thresholds for the preferred polarity than the non-preferred polarity suggesting perception was more likely driven by the preferred polarity. A logistic GLME applied to the data aligned to perceptual threshold, corroborated these results (see FIG. 12I) with the probability of detecting an ECAP increasing with stimulation amplitude relative to perceptual threshold (OR=2.71 per mA), pulse width (OR=1.01 534 per μS), and preferred polarity (OR=1.93 per polarity).

### Factors Predicting N1 Latency

Finally, ECAP N1 latency was analyzed. Unfortunately, artifacts distorted N1 components in some sessions and not all ECAPs had a detectable N1 component. Some ECAPs with N1 latencies were removed that were unrealistically fast (>100 mm/ms, 15 waveforms) or slow (<539 12.5 mm/ms, 5476 waveforms) from further analysis. Overall, N1 components were detected in 540 86.9% of individual ECAPs (n=220,081).

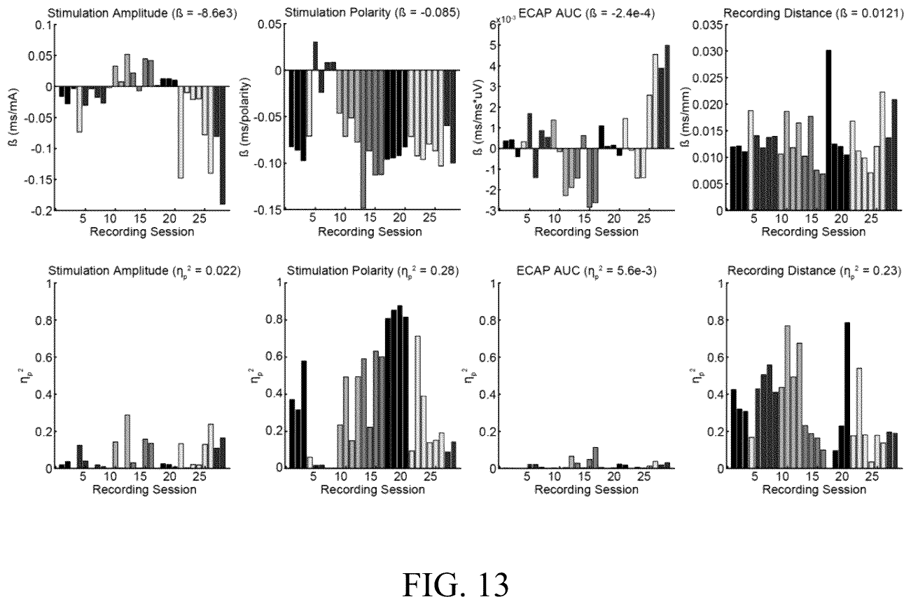

N1 latency in individual sessions was predicted using linear models to mitigate distortion from correcting for lead migration (see FIG. 13). AUC was used as a predictor to account for waveform amplitude effects caused by different stimulation amplitudes, pulse widths, and polarity. It was found that the biggest predictors of N1 latency were recording distance (median β=0.0121 545 ms/mm or 82.7 m/s, median ηₚ²=0.23) and stimulation polarity (median β=−0.085 per 546 ms/polarity, median ηₚ²=0.28). Importantly, N1 latency was slightly lower for 7−/8+ than 7+/8−, and these stimulation polarity effects were consistent across sessions. Stimulation amplitude 548 (median β=−8.6e3 ms/mA, median ηₚ²=0.22) had a small effect on N1 latency while ECAP AUC (median β=2.4e-4 ms/μV*ms, median ηₚ²=0.0056) had no effect on N1 latency.

Next, a group-level LME was built with random intercepts for each session because the individual session linear models may have overestimated conduction velocity. Overall, relatively consistent results were found between the linear models for individual sessions and the group-level LME. Recording distance (8=0.0142 ms/mm or 70.4 m/s, ηₚ²=0.35) was found to have a large effect on N1 latency while stimulation polarity (β=−0.065 per ms/polarity, ηₚ²=0.16) had a medium effect. Again, N1 latency was slightly less for 7−/8+ than 7+/8− by about 0.129 ms. Stimulation amplitude (β=2.e-3 ms/mA, ηₚ²=2.4e-4) and ECAP AUC (β=−5.7e-5 msμ/V*ms, 557 ηₚ²=4.0e-5) had no effect on N1 latency. Results were similar when using LMEs with random slopes for each session (data not shown), and group-level average conduction velocity was estimated as 75.6 m/s and the stimulation polarity effect (β) was estimated as −0.064 ms/polarity.

FIG. 13 shows factors predicting ECAP N1 Latency during individual sessions. FIG. 13 top row shows beta weight estimates from linear models for stimulation amplitude, stimulation polarity, ECAP AUC, and recording distance from the stimulation site. FIG. ### bottom row shows the same as top row but partial eta-squared (ηₚ²) indicating the effect size. Linear models were fit to individual recording sessions where all stimulation pulses had the same pulse width. Values in parentheses in titles are the median estimate across sessions. Each participant has a different shading.

### Discussion

### Box 1: Methods Summary

### 1. Waveform Processing: Artifact Removal and Feature Extraction:

Extract the raw ESR waveforms.
Apply an exponential or polynomial fitting function to model and then subtract stimulation artifacts, ensuring minimal residual contamination.
Extract waveform features such as AUC, P2P, average slope, average voltage, and N1 latency.

### 2. Classification of Waveforms:

Use PCA and k-means clustering to label waveform clusters.
Employ a KNN classifier trained on labeled data to classify waveforms as non-ECAPs, ECAPs, or outliers.
Use ROC analysis to determine optimal thresholds and identify the best computationally efficient feature(s) that discriminate between non-ECAPs and ECAPs as well as ECAPs and outliers.

### 3. Group-Level Evaluation of Stimulation Parameters:

Analyze the effects of stimulation amplitude, pulse width, and polarity on ECAP detection probabilities using logistic GLMEs.
Use LMEs to determine how stimulation parameters affect N1 latency.

### 4. Personalization, Tuning, and Optimization:

Build personalized regression models from brief testing across stimulation parameters using group-level models to provide parameter guidance.
Use perception thresholds as a reference point for understanding ECAP detection probabilities.
Choose personalized stimulation parameters and identify the “preferred” stimulation polarity that reduces the current required to detect ECAPs (E50 values), minimizing battery usage while maximizing efficacy.

In this study, a systematic method for processing and analyzing ESR during SCS was described using different features with a combination of machine learning approaches to determine the best features for discriminating between non-ECAPs, ECAPs, and outliers. Regression-based statistical models were then applied to determine how these features relate to stimulation parameters and vary with recording distance. Finally, optimal thresholds were applied of the best features to detect ECAPs and outliers and determine how ECAPs and outliers were related to stimulation parameters and vary with recording distance. These methods and results are critical to the future development of personalized, closed-loop applications.

### Results Summary

First, PCA, k-means clustering, KNN classifier, and ROC analysis were combined to determine the best computationally efficient feature for discriminating between non-ECAPs, ECAPs, and outliers. AUC was found to be the best feature for discriminating between non-ECAPs and ECAPs while PC1 was the best feature for discriminating between ECAPs and outliers. From the ROC analysis, AUC was found better than P2P (Non-ECAPs vs ECAPs=2.44 vs 2.27) at detecting ECAPs even though P2P is the most commonly used feature. As expected, ECAP and outlier discrimination was generally easier (d′>2) for features that correlated with capacitive artifact shapes: PC1, average voltage, and average slope. The ROC analysis also extracted optimal thresholds for each feature using Youhen's J-statistic which can then be applied directly and efficiently to the data without needing to use more computationally expensive methods like PCA and KNN classifiers.

Second, optimal thresholds were applied for average voltage and average slope to detect outliers and the optimal threshold for AUC to detect ECAPs. Logistic GLMEs were built to understand the relationship between stimulation parameters, recording distance, and the probability of detecting an outlier or ECAP. As expected outliers were more common at recording sites near the stimulation electrodes (OR=0.86 per mm), at higher stimulation amplitudes (OR=1.60 per mA), and for wider pulse widths (OR=1.006 per μS); stimulation polarity had only a small effect on the probability of detecting an outlier (OR=0.93 per polarity).

Third, a logistic GLME modeling the probability of detecting ECAPs found that stimulation amplitude (OR=2.65 per mA), pulse width (OR=1.01 per μS), and polarity (OR=0.64 per 625 polarity) strongly predicted the probability of detecting an ECAP. Due to the small decrease in amplitude of evoked response at distal recording sites, there was also a small effect of recording distance (OR=0.93 per mm) on the probability of detecting an ECAP. Interestingly, when individual participants were analyzed, inter-participant variability was found in the probability of detecting ECAPs across different stimulation polarities. In essence, certain participants appeared to have preferred stimulation polarities, so a new GLME was built using these preferred polarities as a predictor. The new GLME found that E50 was ˜1.25 mA less for the preferred polarity compared to the non-preferred polarities indicating ˜1.25 mA less current was needed to produce ECAPs for the preferred polarity. The probability of detecting an ECAP was also modeled relative to perception threshold, and it was found that ECAPs were only detected at stimulation amplitudes greater than perception. Perception was also likely driven by the preferred polarity.

Finally, ECAP N1 latency was analyzed using linear models and LMEs in individual sessions and at the group-level, respectively. Importantly, N1 latency calculated across the span of the recording electrodes can be used to estimate conduction velocity. As with the other evoked waveform features, it was asked if there was a relationship between N1 latency, stimulation amplitude, stimulation polarity, and recording location. As expected, recording distance was the strongest predictor (ηₚ²=0.35) of N1 latency and conduction velocity was estimated to be 70.4 m/s at the group-level though conduction velocity varied slightly across individual participants. This conduction velocity value is consistent with other studies in humans and animals. Surprisingly, a small (0.129 645 ms) but consistent effect (ηₚ²=0.16) of stimulation polarity on N1 latency was found. This latency effect of stimulation polarity was equivalent to moving the effective stimulation site 9.8 mm (70.4 mm/ms*0.129 ms).

### No Single Feature can Completely Account for the Evoked Response Classification

It was consistently found that AUC was the best feature for predicting ECAPs (see Table 2, FIGS. 10G-I). While AUC is correlated with P2P, AUC accounts for additional waveform energy components including peak widths (i.e., dispersion) and the presence of smaller peaks which can occur in some participants. Since AUC is a simple, computationally efficient amplitude feature like P2P, AUC could easily replace P2P in real-time applications.

Like all features, P2P and AUC derived from electrically evoked responses are prone to stimulation artifacts. This work herein demonstrates that AUC is not the best for outlier classification (see Table 2) which is not surprising since PC1, average slope, and average voltage describe partial or whole components of the stimulation artifact related to electrode and tissue impedances. Here, the outlier class mostly describes artifacts or evoked responses with insufficient artifact removal (see FIG. 10). While PC1 is more mathematically complicated, average voltage and average slope are simple, computationally efficient metrics that could be used in real-time applications. Overall, these results suggest the need for separate features for different classes, and in the end, no one feature appears to be universally the best.

Interestingly, the number of detected peaks and valleys using findpeaks.m did a poor job compared to the other features at discriminating between classes (see Table 2, d'Non-ECAPs vs ECAPs=668 1.23 & d'ECAPs vs Outliers=1.62). It is possible that peak/valley detection is unnecessary to begin with as the stimulation artifacts could also generate detectable peaks that look like ECAPs. The fact that N1 in ˜87% of ECAP waveforms was reliably detected suggests that individual waveforms are not too noisy for accurate peak detection. However, a comparison of averaged waveforms across multi-second stimulation periods may be necessary to verify this.

### The Utility of Classifying and Predicting Evoked Responses

To implement a closed-loop stimulation system, accurate prediction of evoked responses and their class across various stimulation parameters is important. The first consideration, that is often overlooked, is the ability to separate responses from outliers. Many previous research works quantify evoked responses without first predicting if an evoked response exists. The results herein suggest the use of a single simple feature, such as P2P and AUC, alone for closed-loop clinical applications could be problematic because these features' values increase with certain stimulation parameters even when an ECAP is not present. If there is no evoked response, values derived from these features should not be used in closed-loop control as it simply represents the amplitude of an artifact and not a biological signal. This issue highlights the importance of waveform classification and using feature prediction methods to separate non-ECAPs, ECAPs, and outliers.

This example shows that simple logistic GLME models can reliably predict different waveform classes and how different stimulation parameters influence the probability of detecting these classes. The outlier logistic GLME results indicated that outliers were more probable at recording sites near the stimulation electrodes, at higher stimulation amplitudes, and at longer stimulation pulse widths (see FIG. 11). Specifically, the GLME model suggested that recording sites within 15 mm (from center of contacts 7/8) were too close to reliably detect ECAPs due to the high prevalence 692 (>10%) of outliers especially at higher stimulation amplitudes and longer pulse widths. These outlier results are consistent with the observation from a previous study on animal participants. A different logistic GLME predicting the probability of observing an ECAP showed similar results. ECAPs were more probable at higher stimulation amplitudes and at longer stimulation pulse widths. ECAP detection was also slightly more probable on proximal recording electrodes than distal recording electrodes due to the slight decrease in ECAP amplitude as the ECAP travels rostrally through the spinal cord. While these results are consistent with previous studies, the results herein demonstrate that the same factors that predict the probability of detecting an ECAP also predict the probability of observing an outlier. Ideally, low stimulation amplitudes, short stimulation pulse widths, and signals on distal recording electrodes would be used in closed-loop applications to avoid the influence of stimulation artifacts, but clinically this may not always be feasible as sufficiently high stimulation amplitudes are needed to produce ECAPs. Thus, it is important to be able to classify evoked responses as outliers or ECAPs to prevent detrimental adjustments in closed-loop applications if outliers are mistaken for ECAPs.

A similar approach was taken to better understand how different stimulation parameters predict evoked response features in a continuous manner. The goal of closed-loop applications is to adjust the stimulation parameters in order to maintain an evoked response within a certain signal amplitude range. Thus, it is critical to understand which parameters are necessary to observe a particular class of responses but also to quantify how different stimulation parameters influence feature values of those responses. By building a statistical model offline using many individual waveforms (see FIGS. 11, 12 and Table 3), the expected evoked response feature values with different stimulation parameters can be predicted. In particular, AUC, P2P, and N1 amplitude features correlated strongly with stimulation amplitude, and these results show stimulation amplitude has the largest effect on these features followed by pulse width and recording distance (see table 3). These results suggest that regression models built with these features captured the relationship between stimulation parameters and evoked responses well. These models could be used to predict evoked response features a priori to help determine appropriate clinical stimulation parameters in closed-loop applications.

The statistical modeling results herein mostly focused on group-level data, but individual models for individual participants were also created. Group-level models are useful for predicting responses in new and untested individuals, but models tailored to individuals are important for delivering effective personalized stimulation. The individual models presented here were made from single session data from less than or equal to 20, 10 second stimulation periods which were obtained in less than 7 minutes. These individualized models could be useful in rapidly optimizing closed-loop applications.

### The Importance of Stimulation Polarity

Stimulation polarity had clear effects on AUC and the probability of detecting an ECAP. Increases in AUC for preferred polarity, which varied by participant, led to ECAPs being observed at lower stimulation amplitudes than for the non-preferred polarity. These effects were also observed in the minimally processed data (see FIG. 19) suggesting this effect is not an artifact of this analysis or the optimal AUC threshold. Importantly, stimulation amplitudes for the preferred polarity required ˜1.25 mA less current than the non-preferred polarity to evoke the same response. Consistent with other studies, these data suggest there is potential for significant energy savings simply by stimulating participants with their preferred polarity.

Unfortunately, the underlying mechanism driving these preferred polarity effects could not be investigated further because there was substantial evidence of relative lead migration. However, other work suggests that the polarity effect may be due to the electrode locations relative to the dorsal rootlets as well as anatomical variability. Future work with contemporaneous imaging will need to confirm this observation.

While the preferred stimulation polarity effects on evoked response amplitudes varied across participants, there was a more consistent effect of stimulation polarity on N1 latency. This small effect translated to an estimated 9.8 mm shift in the effective stimulation location. Since the center-to-center spacing of contacts on the Octrodes™ lead is 7.0 mm, this effect would suggest that the effective stimulation location moved caudally approximately 1 electrode with the cathode. Essentially, ECAP activation occurred near the cathode rather than the anode as predicted by neuromodulation models. However, it is unclear what factors may contribute to additional ˜2 mm discrepencay. First, the estimation of conduction velocity may be error prone due to vertical lead migration and artifact distortion of N1. Lateral lead migration could also not be accounted for. Second, the action potential initiation site may not strictly occur under the cathode, as the changing tissue-electrode interface and underlay cerebrospinal fluid could vary the delivered current pathway, and the anodic phase of the stimulation could also trigger action potentials as well. Third, there may be additional mechanisms that are currently unknown.

### Limitations, Other Considerations, and Alternate Approaches

Data was analyzed on individual channels, but multiple recording electrodes are available to capture responses and provide corroborating or complementary information which might improve quality and safety in the design of a closed-loop neuromodulation system. However, waveform features potentially vary with anatomy and distance from the stimulation site (e.g. AUC 767 decreases). Due to this variation, closed-loop applications synthesizing data from multiple electrodes will need to account for these complexities in order to improve robustness.

While the analyses shown here were applied to individual evoked responses following single stimulation pulses, similar analyses could be applied to averaged evoked responses over many stimulation pulses. For averaged signals the same measures should be at least as good because averaging reduces noise. In particular, lower feature thresholds could be used for classification with averaging than with individual evoked responses. Lower thresholds could help detect ECAPs closer to the perception threshold where only very small ECAPs (e.g. P2P<5 μV) are commonly observed (e.g., see FIG. 8D). Averaging may be crucial as 46.5% of waveforms were collected at less than 1 mA above perception threshold but on average 68.7% of waveforms were classified as non-ECAPs. This result suggests this method is not sensitive enough to capture small ECAPs though some waveforms were lost to artifacts as well. An alternative strategy would be to combine classification of individual waveforms with averaging across a small number of waveforms (e.g., over a 1 second period) to first remove outliers, and then quantify averaged ECAP waveforms thus combining the best of both methods to improve the signal-to-noise ratio for closed-loop applications.

At the same time, averaging across different stimulation parameters, such as stimulation polarities, could warp the evoked response producing ECAPS with different latencies and amplitudes. Additionally, stimulation effects, like those for preferred polarity, can vary across participants presumably due to change in the relative location of stimulation electrodes to the dorsal rootlets. The main benefit of averaging across stimulation polarity, for example when switching polarity on a pulse-by-pulse basis, is that the stimulation artifacts will theoretically cancel out. Some mathematical models could help account for stimulation parameter effects on evoked responses which would allow recovery of the true ECAP component while still using such a simple artifact cancellation scheme though these methods may have to account for complicated nonlinearities.

### Conclusions

This study successfully employed a combination of machine learning techniques, including Principal Component Analysis (“PCA”), k-means clustering, a K-Nearest Neighbors (“KNN”) classifier, Receiver Operating Characteristic (“ROC”) analysis, and regression-based models to analyze evoked responses from spinal cord stimulation (“SCS”). These findings reveal that the Area Under the Curve (“AUC”) effectively differentiates between non-ECAPs and ECAPs, while average voltage and slope are particularly useful for differentiating ECAPs from outliers. These computationally efficient features are ideally suited for real-time applications in implantable pulse generators (“IPGs”) for online classification, enhancing the functionality of future closed-loop systems. This work also emphasizes the importance of classifying evoked responses in addition to quantification of the ECAP, which is an important step currently missing in closed-loop SCS systems.

This research also highlights significant inter-participant variability in response to SCS, emphasizing the need for personalized stimulation parameters to optimize treatment efficacy and improve energy efficiency. For instance, variations in the amount of current required was observed to evoke ECAPs based on stimulation polarity across different individuals, suggesting that personalization could lead to more effective and energy-efficient treatments. In conclusion, this work provides a feasible framework for describing the relationship between evoked responses and stimulation parameters, which will be useful for predicting the presence of evoked responses before adjusting stimulation in a closed-loop application for SCS.

FIG. 14 shows lead migration and post hoc electrode alignment based on N1 latency. FIG. 14A shows a schematic of method for post-hoc re-alignment using a simple regression method based on N1 latency. The relative rostral-caudal position of the right lead was programmatically adjusted, then the N1 latency against rostral-caudal (RC) position regressed, and then the MSE was calculated. The relative position of the right lead that minimized the MSE of the regression of N1 latency versus RC position was chosen. FIG. 14B shows averaged ECAP waveforms for the left lead (green, channels 2-6) and right lead (red, channels 10-16) for stimulation amplitudes that elicited ECAPs plotted with respect to their Rostral-Caudal (“RC”) location relative to stimulation electrodes 7/8. Peaks and valleys were detected with findpeaks in Matlab. Only channels with detectable peaks and valleys were included. Note for this participant channel 1 was used as a ground electrode. FIG. 14C shows averaged ECAP waveforms for contacts on the left (green) and right (red) leads using alignment based on postoperative X-ray positions. FIG. 14D shows averaged ECAP waveforms for contacts on the left (green) and right (red) leads using alignment based on N1-latency corrected position. The peak and valley times and amplitudes match better for the N1-latency corrected alignment than for the postoperative X-ray based alignment. FIG. 14E shows N1 latency corrected times plotted against their relative RC position. The slope (m) indicates the estimated conduction velocity of the ECAPS based on the N1 latency as a function of position. FIG. 14F shows N1 conduction velocity across recording sessions and participants (different shading). FIG. 14I shows predicted lead migration across sessions and participants (same shading as H) based on N1 latency corrected position. Note, estimated lead migration appeared relatively stable across sessions. Predicted lead migration is only based on relative lead migration between left (contacts 1-6) and right (contacts 10-16) leads and cannot be used to predict absolute lead migration relative to the spinal cord. Also, note, conduction velocity estimates using this methodology were higher than the formal analysis likely due to distortion of the ECAPs waveforms when averaging across stimulation polarity and the inclusion of contacts close to the stimulation sites (e.g., contact 6).

FIG. 15 shows visualization of PCA and correlation of PCA scores with waveform features for the same example outer loop data in FIG. 10. FIGS. 15 A and B show average waveform characteristics as indicated by PCA (A) and waveform amplitude (B) varied greatly across clusters determined by k-means clustering. Note, waveform P2P amplitude was not used during clustering but only to label clusters. FIGS. 15C-E) show visualization of the 1st 3 PC by taking the average of waveforms sorted by every 10th-percentile for each PC score. FIGS. 15F-H show average correlation (r) of the 1st 3 PC with waveform features. While PC1 was strongly correlated with average voltage and average slope, PC2 and PC3 appeared correlated with various peaks and valleys instead of any particular waveform feature.

FIG. 16 shows a distribution of waveform features and PC scores by KNN classifier results for the same example outer loop data in FIG. 10. Distribution of different waveform features and PC scores separated by waveform class and low vs high-confidence classification. The magnitude (∥) of the average voltage, average slope, and PC scores was used to create one tailed distributions; these distributions appeared symmetric when using raw values.

FIG. 17 shows the effect of stimulation parameters on waveform features. All plots median+/−IQR calculated across all other predicted values. Data does not include outliers, and the number of observations is not equal across participants. For N1 amplitude, only waveforms with a detectable N1 were used.

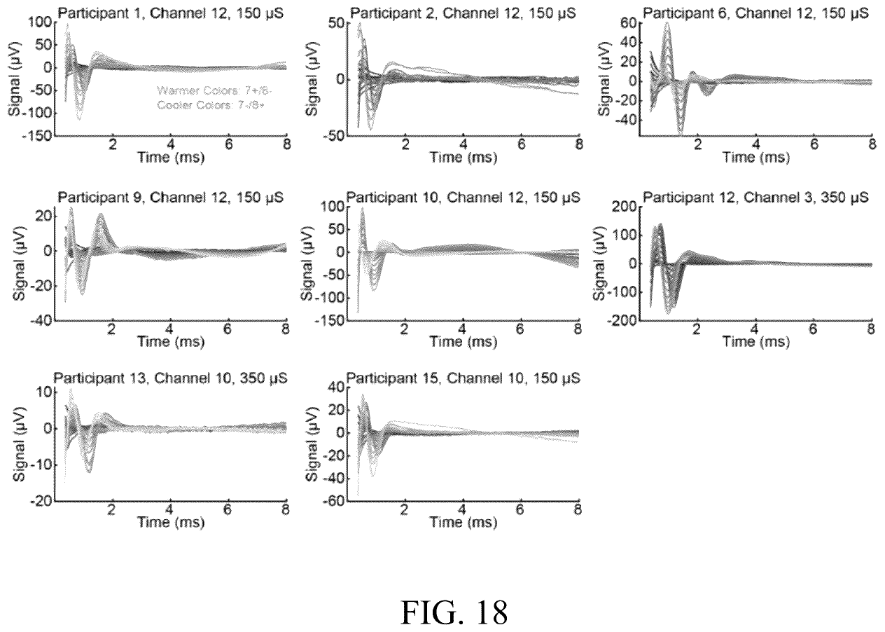

FIG. 18 shows an example averaged evoked responses across participants. Example average evoked artifact-cleaned waveforms for each participant on an example channel for each stimulation polarity (warmer vs cooler colors) across different stimulation amplitudes. Other than removing the artifact and averaging, no other processing was done.

FIG. 19 shows the probability of detecting an ECAP relative to perception threshold. FIG. 19A shows the probability of detecting an ECAP for each participant on their most sensitive channel for each pulse width and for preferred versus non-preferred polarity. The probability of detecting an ECAP for preferred polarity was consistently closer to the perception threshold than the non-preferred polarity. Additionally, in all participants, ECAPs were only detected above the perception threshold. FIG. 19B shows an example averaged evoked responses for Participant 9 on channels 2 and 3 at different stimulation amplitudes relative to perception threshold. ECAPs were clearly observed at 4 mA above the perception threshold and not at 2 mA below perception threshold. At perception threshold small deviations on channel 2 (P2P˜5.5 μV) and channel 3 (P2P˜7.7 μV) were observed. However, individual waveforms were not classified as ECAPs. Furthermore, artifacts remnants, especially on channel 3, indicate that at least some parts of these waveforms may be artifacts.

### Example 2

Example 2 provides systems and methods towards a Low-cost Online Application: a Curve Fitting-free Artifact Rejection Approach for Processing Evoked Compound Action Potential during Spinal Cord Stimulation

### Introduction

Spinal cord stimulation (SCS) is used to treat chronic pain in 50,000 patients a year. While electrical stimulation applied through the spinal cord has been shown to be effective, the mechanism is not fully understood. A complication with SCS is that the spinal cord moves relative to the SCS electrodes depending on the position of the patient. When a patient is supine, the spinal cord will rest on the electrodes, and stimulation amplitude is turned down for comfort. When standing or prone, the spinal cord moves away from the electrodes and the amplitude must be increased to maintain efficacy. One approach to adjusting the stimulation automatically is to adjust amplitude according to the orientation of the patient, measured by accelerometers placed on the IPG. Another approach is to measure the evoked response of the spinal cord to the electrical stimulation with the assumption that stimulation must evoke a response to be effective. If the evoked response is too small then the amplitude can be increased, if it is too large, then the amplitude can be decreased. Closed-loop adaptive feedback mechanism that adjust stimulation based on the amplitude of the evoked responses has been found to keep patients at an effective stimulation amplitude for a greater proportion of the day than open-loop stimulation or positional control.

Epidural spinal recordings (ESR) are captured using leads placed in the epidural space usually from electrodes that are near the stimulating ones. ESRs contain multi-modal response such as the evoked compound action potential (ECAP), the evoked muscle response, stimulation artifact, etc. The stimulus artifacts may be hundreds of times larger and can take several milliseconds longer to recover than the ECAP, which can severely distort and contaminate the ECAP component in ESR. Thus, efficient, and robust stimulation artifact rejection is an important component to measuring ECAPs in real-time. Minimizing the computational requirements of the signal processing algorithm is pivotal for an embedded system.

Stimulus artifact reduction can be accomplished with a combination of hardware and software methods. Amplifiers measure the potential difference between a working electrode and a reference electrode. In theory, if the working electrode and reference both see volume conduction of the stimulus artifact with similar amplitudes, the artifact can be effectively removed. But placement of the reference electrode where it can detect neural signals can result in a signal that does not reflect the underlying neurophysiological response. Another approach is to blank the amplifier during stimulation by shorting the inputs to prevent charge buildup, which results in long transient discharge causing stimulus artifacts. While there are some dedicated hardware systems that can blank the stimulation artifact, most recordings using separate stimulators and amplifiers do not have blanking capabilities. However, even with blanking, there can be a significant remaining artifact, especially at short latencies.

There are software approaches to removing the artifact as well, particularly in the cases when the stimulus does not saturate the amplifier. Linear filters, such as analog filters or digital filters, cannot sufficiently capture the dynamic change of the stimulation capacitive artifacts and create filtering artifacts in the processed signal that corrupts the data for further analysis. Another approach is to average responses while alternating the stimulation between anodic leading to cathodic leading so that the linear summation of the resulting waveforms will average out the stimulus artifacts, which are mostly linear, while the nonlinear neural response will remain. However, anodic and cathodic leading stimulation has been found to evoke neural responses at different latencies and amplitudes producing, summed waveform that does not accurately reflect the ECAP. More advanced filtering techniques, such as independent component analysis, stimulation masks based baseline removal techniques, and Savitzky-Golay filtering, can do a better job. Curve fitting a function, like a linear plus an exponential curve or a double exponential curve, to the stimulus artifact and subtracting it can be very effective at removing artifacts. However, no curve fitting is perfect, and a poor fit can introduce artificial features in the processed signal that may be difficult to disambiguate from a real neural response.

Further complicating the stimulus artifact removal, the ESR is not stationary and the artifact may change with changes in tissue-electrode impedance, body posture, and lead position. Thus, a pre-fitted baseline curve cannot capture the dynamically changing nature of the stimulation artifact in the recording.

In this disclosure, the feasibility of a low-cost signal processing pipeline is explored to make it more achievable on an embedded system. This new method is termed the Spatial and Amplitude Normalized Difference (SAND) ECAP, which is a Template Subtraction method. The computational time of the proposed method was compared to one commonly used curve-fitting based method. In addition, the evoked dose-response curve was compared from eight human subjects implanted with trialing leads prior to permanent implants for treatment of chronic pain. This shows how ECAPs change with sensory perception threshold and compare the advantages and disadvantages using different artifact rejection approaches and their accuracies with respect to predicting perceptual thresholds.

### Methods

### Participants

Spinal cord recordings were measured in eight patients implanted with trialing percutaneous spinal cord stimulation electrodes for treatment of chronic pain were (Table 4). The temporary leads were implanted and externalized to connect to an external pulse generator for assessing efficacy of the therapy to determine if these patients are candidates for permanent implants. All participants were consented in accordance with a University of Minnesota TRB (00013100) approved protocol which was registered on ClinicalTrials.gov (NCT04938245).

TABLE 4
Participant Demographics

| | | | | Electrode | Stim/Record | | |
|---|---|---|---|---|---|---|---|
| ID | Sex | Age | Pain Region | Placement | System | Ethnicity | Race |
| 1 | M | 56 | right leg and back | T8-T10 | ATLAS/ATLAS | not | white |
| | | | | | | 63ispanic | |
| | | | | | | or latino | |
| 2 | M | 53 | Abdominal | T6-T8 | ATLAS/ATLAS | not | white |
| | | | (CRPS) | | | 63ispanic | |
| | | | | | | or latino | |
| 3 | M | 46 | back pain and | T7-T9 | TDT/TDT, | not | white |
| | | | diabetic | | ATLAS | 63ispanic | |
| | | | neuropathy | | | or latino | |
| 4 | F | 68 | neck and upper | C2-C5 | TDT/TDT, | not | white |
| | | | back between | | ATLAS | 63ispanic | |
| | | | shoulders | | | or latino | |
| 5 | F | 57 | between | T2-T4 | TDT/TDT | not | white |
| | | | shoulders | | | 64ispanic | |
| | | | | | | or latino | |
| 6 | M | 37 | lower back and | T7-T9 | TDT/ATLAS | not | white |
| | | | legs | | | 64ispanic | |
| | | | | | | or latino | |
| 7 | F | 72 | lower back and | T7-T9 | TDT/ATLAS | not | black/ |
| | | | legs | | | 64ispanic | 64ispani |
| | | | | | | or latino | american |
| 8 | M | 50 | lower back and | | TDT/ATLAS | not | white |
| | | | legs; Spina bifida | | | 64ispanic | |
| | | | | | | or latino | |

### Protocol

The participants were implanted with two percutaneous leads each (Octrode™ Abbott, Austin, TX) with a total of 16 electrodes. Surgery details are discussed in previous papers. The placement of the two leads were chosen for clinical therapy. In most cases, the two leads were on the same rostral-caudal level with an offset of 0-2 electrodes. The participants were tested over 1-3 visits during the trial period of electrical spinal cord stimulation. One participant was also tested in the operating room.

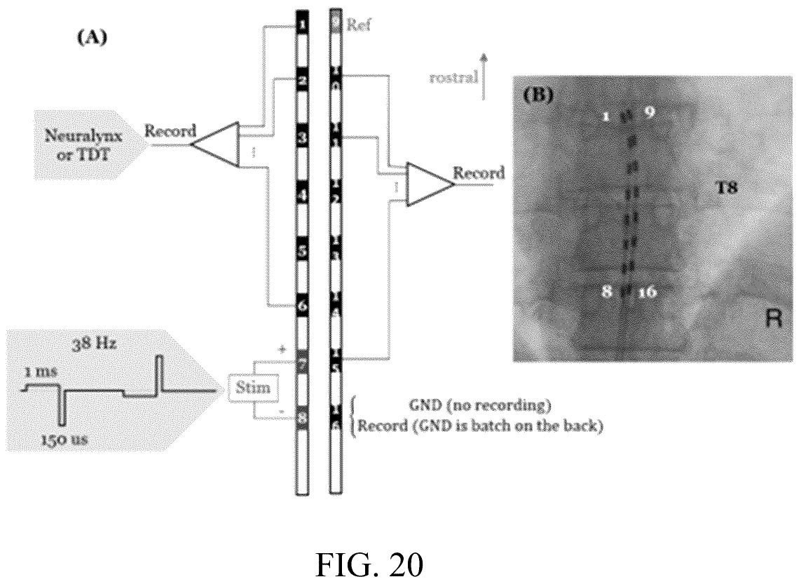

As summarized in FIG. 20, electrodes #7 and #8 were assigned to the bipolar stimulation delivery. Electrode #9 was connected to the recording reference. Electrode #16 was used as a ground in participant #2, while the other participants had the ground as a separate patch electrode on the skin on the participant's back. The remaining lead electrodes were used for recording.

Asymmetric biphasic charge-balanced tonic stimulation was used to evoke ECAPs. The stimulation waveform was programmed to have a 1-ms charge-balancing phase followed by a 150-us pulse. The pulse train for a given stimulation amplitude ran for 10 seconds at 38 Hz. The stimulation amplitude was ramped up in small steps starting from a sub-threshold amplitude until reaching a discomfort limit of each participant (e.g., 0.5, 1, 1.5, . . . , 6 mA). Each amplitude applied for at least one trial of 10 seconds. Participant-reported paresthesia was recorded as well. Two stimulations polarities were used: cathode on 7 and cathode on 8. The stimulation generator was either an ATLAS Stim headbox (Neuralynx, Bozeman, MT) or an IZV10 and subject interface module (SI-8, Tucker-Davis Technologies, Alachua, FL) with clocks of 40 kHz and 24 kHz, respectively.

Data was recorded using either an ATLAS neurophysiology system (Neuralynx, Bozeman, MT) or a PZA on SI-8 with RZ5D processor (Tucker-Davis Technologies, Alachua, FL). The sampling rates were 32 kHz and 24 kHz, respectively. The leads were plugged into an adapter which is then connected to a Medusa cable with touch-proof ends connected into the stimulator and the recorder (Neuralynx headbox or TDT SBOX16).

FIG. 20 shows the experiment layout. FIG. 20(A) shows two Octrode™ leads with a total of 16 electrodes were implanted. The stimulation waveform was delivered to electrodes 7 and 8 as asymmetric biphasic pulses at 38 Hz. The recording electrodes were connected to a Neuralynx or a TDT device. FIG. 20B shows an example X-ray from participant #7. Ref is the recording reference electrode. Stim is the stimulator.

### Data Processing

Recordings were divided into blocks of the same stimulation amplitude and polarity per channel. Each block was filtered with a 100-ms median filter (MATLAB's medfilt1) to remove any baseline drift. Stimulus onset was measured on channel 6 and time zero for the stimulus waveform was set at the trailing edge of each pulse to align recordings across the different stimulation pulses. Ensemble average of each channel was computed. To minimize stimulation and far-field noise artifacts, a spatial differential was used for channels (Ch) on the same lead in the form Ch(i+1)−Ch(i) where i=1, 2, . . . , 16, e.g., Ch(2)−Ch(1). Data analysis was done in MATLAB R2021b (MathWorks, Natick, MA).

### Novel Artifact Removal Method: AND/SAND ECAP

The classical approach of artifact removal is to fit a function to the ensemble average in a time window after the pulse then subtract the fit from the signal. In previous studies three common functions were compared: a single exponential (exp1), a double exponential (exp2), and a second order polynomial (poly2) fit to a window of data from 0.375 ms to 4 ms. Poly2 had similar ECAP metrics yet significantly shorter computation time compared to exp2. The computation time/effort is an important factor for an embedded eSCS system utilizing ECAP as a feedback signal. Therefore, a method with lighter computation resources is pivotal for an embedded closed-loop stimulator.

Here a novel method to remove artifacts and extract spinal-cord ECAP without fitting: the Amplitude Normalized Difference (AND) method is presented. The method uses the ensemble average from two stimulation amplitudes. Since the artifact originates from the impedance properties of the electrodes, recording hardware and tissues, the artifact dynamics are mostly linear (FIG. 2). Therefore, normalizing each response by its excitation magnitude scales the artifacts to have the same magnitude. Second, a differential is taken between the normalized signals to remove this scaled linear response (the artifact) and leaves any nonlinear responses, presumed to be of neural origin. If the method uses the channel spatial differential, ch(i)−ch(i+1), then output was termed as the Spatial and Amplitude Normalized Difference (SAND) ECAP, which is used throughout the results presented here.

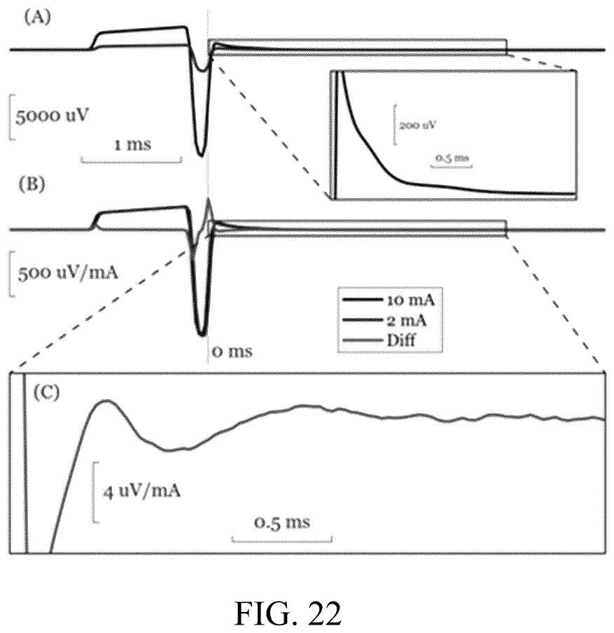

The idea behind (S)AND is that stimulus artifacts generally scale linearly with stimulation amplitude (unless they reach nonlinearities caused by range limits of the amplifier), and neural responses scale nonlinearly. To represent the (S)AND method mathematically, assume the recorded response is given by y=Gu+f(u), where the signal y is the measured response and u is the stimulus waveform, G is the linear dynamics from the input to the output, which it was expected to be the stimulus artifact, and f(⋅) is a nonlinear response, which it was expected to be from neural origins. Now consider two inputs with the same waveform but with different amplitudes, α and β, and their responses y₁=αGu+f(αu) and y₂=βGu+f(βu). The amplitude-normalized responses are y₁/α=Gu+f(αu)/α and y₂/β=Gu+f(βu)/β. The difference between these two amplitude-normalized responses is y₁/α−y₂/β=f(αu)/α−f(βu)/β. As can be seen, the linear dynamics are canceled, and thus local non-linear response with stimulation amplitude change was observed. FIG. 22 explains these processing steps in one dataset from participant #6 with two stimulation amplitudes of 10 mA (above ECAP threshold) and 2 mA (below ECAP threshold).

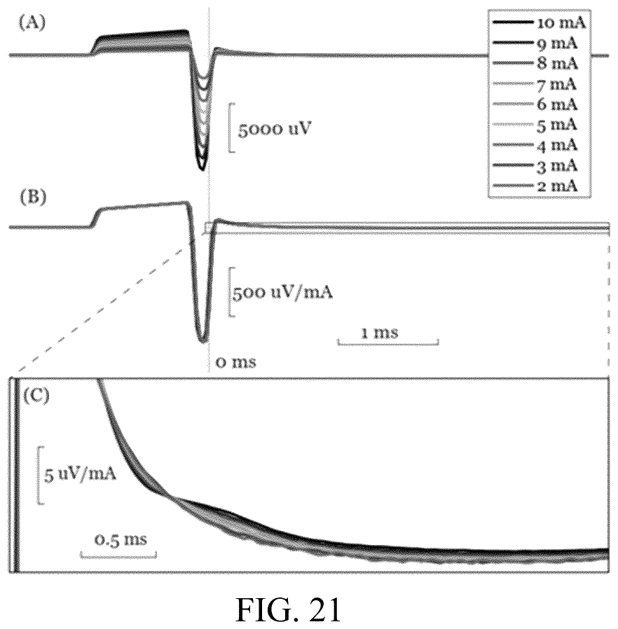

FIG. 21 shows the artifacts are the same size in the normalized recordings. This dataset is from ch(11)-ch(12) in participant #6 in the second visit during cathode-on-8 stimulation at different stimulation amplitudes. The mean recordings are FIG. 21(A) un-normalized and FIG. 21(B) normalized by the stimulation amplitude. FIG. 21(C) shows small ECAPs ride on huge stimulation artifact. The normalized artifacts are very similar between different stimulation amplitudes, reflecting a linear response. This motivated the new S(AND) method to remove the artifacts in spinal-cord ECAPs.

FIG. 22 shows the novel artifact removal SAND method using spatial and amplitude differences is illustrated in steps. This dataset is Ch(11)-Ch(12) in participant #6 in the second visit during cathode-on-8 stimulation. FIG. 22(A) shows the mean recorded waveforms from super- and sub-threshold stimulation amplitudes (10 mA and 2 mA, the black and blue lines, respectively). The inset shows ECAPs, at the super-threshold amplitude, riding on an artifact in the time window 0-3 ms. FIG. 22(B) shows the waveforms were normalized by their respective stimulation amplitudes and the difference was taken (the red line). Note the reduction in the artifact amplitude in the difference (red line). FIG. 22(C) shows a zoomed view of the differential signal in the time window 0-3 ms shows clean SAND ECAPs without artifacts. Note this SAND ECAP have the units of uV/mA, which will be further multiplied by the large stimulation amplitude (10 mA in this case) to transform the units to uV.

### ECAP Metrics

The ECAP morphology features a three peak pattern (P1, N1, P2). To determine the peaks effectively, the N1 peak is searched for first then search for P1 preceding it and P2 following it. The peaks were identified using MATLAB's findpeaks function within a predefined time window and a minimum peak height of 0.1 uV. The algorithm starts the peak search for a given channel from the largest stimulation amplitude since it is the most likely to have clear ECAPs. The algorithm then moves to the next smaller stimulation amplitude. If the peak time at the smaller amplitude differs by more than 0.15 ms from larger the amplitude response, or no peak is found, the peak time of the higher amplitude response is used. This inherently assumes ECAPs are produced at the highest stimulation amplitude.

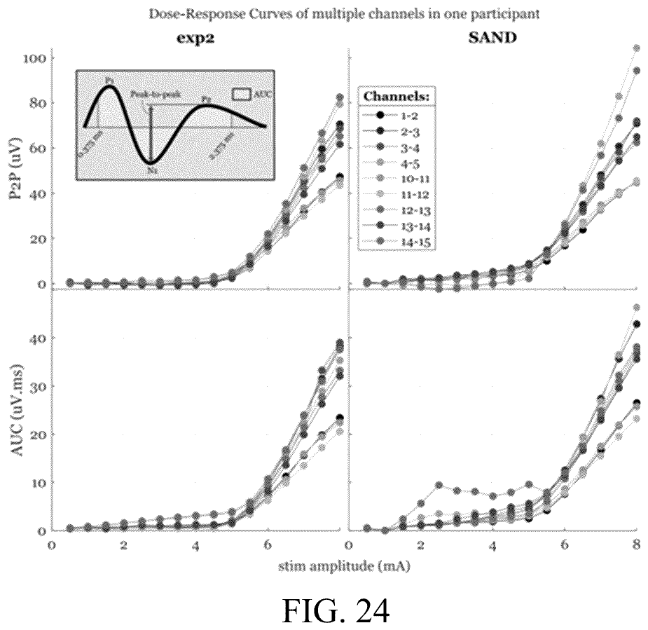

Two ECAP metrics were calculated: the peak-to-peak (P2P) and the area under the curve (AUC). P2P is defined as the difference between the N1 and P2 amplitudes. AUC is defined as the mean-mean corrected under the ECAP curve between 0.375 ms and 2.375 ms. These metrics are shown in FIG. 24A. The dose-response curve (sometimes called the growth curve) is the plot of an ECAP metric versus the stimulation amplitude.

To calculate the ECAP threshold (ET), an iterative two-line fitting method was employed on each data set (participant/visit/polarity/channel/artifact removal method/ECAP metric). First, the data set was divided into two groups: the two smallest stimulation amplitude points and the remaining points. These two groups are candidates for the subthreshold and suprathreshold regions in the dose-response curve. A line was fit to each group, such that their intersection point would be a candidate ET. The sum of the root mean square errors (RMSE) from the two fits was recorded. In the next iteration, the smallest stimulation amplitude point in the suprathreshold group was moved to the subthreshold group to create new candidates. Two lines were fit to the new groups, and RMSE was recorded. The iterations continue until the super threshold group contains only two points. The minimum RMSE among all candidate groups was selected. The intersection between the selected two line fits was taken as the final ET.

### Measuring Significance of Correlation Between ECAP and Perception Thresholds

To determine which method of extracting ECAPs most accurately reflect the perception threshold, the cross-correlation was measured between the ECAP threshold and the perception threshold. To determine if the correlations using the two methods were significantly different, the Fisher's Z-transformation was applied by converting the Pearson correlation coefficients into a variable that approximates a normal distribution (Fisher, 1921). The transformed coefficients were then compared using a Z-test to determine the likelihood of null hypothesis, that both methods have the same correlation.

### Results

The motivation behind the (S)AND method was to make a low computational cost robust stimulus artifact removal algorithm for embedded systems that deliver stimulation and measure neural responses for closed-loop feedback. (S)AND is based on the observation that the stimulus artifact waveforms look very similar in ensemble averages when normalized by their stimulation amplitude (FIG. 21). At higher amplitudes, the ECAP wave can be seen as a small deviation from this stimulus artifact. Moreover, it was found that the estimated artifacts from the double exponential fit were also similar when normalized (data not shown). Therefore, a stimulus artifact removal was sought based on this observation to remove stimulus artifacts with small computation burden.

The (S)AND method is illustrated in FIG. 22 with data collected from a representative participant in our clinical study to measure spinal cord ECAPs. The ensemble averages of the spatial differential Ch(11)−Ch(12) are shown for super- and sub-threshold amplitudes (10 and 2 mA, respectively). In the suprathreshold case, the ECAP wave can be seen as much smaller than the huge stimulus artifact (the top inset). Moreover, the two artifacts' sizes can be clearly distinguished. After normalizing them, they match and become indistinguishable (middle panel). The differential between these normalized signals is much smaller in size (red line, middle panel) and removes the stimulus artifact and reveals the ECAP (bottom panel).

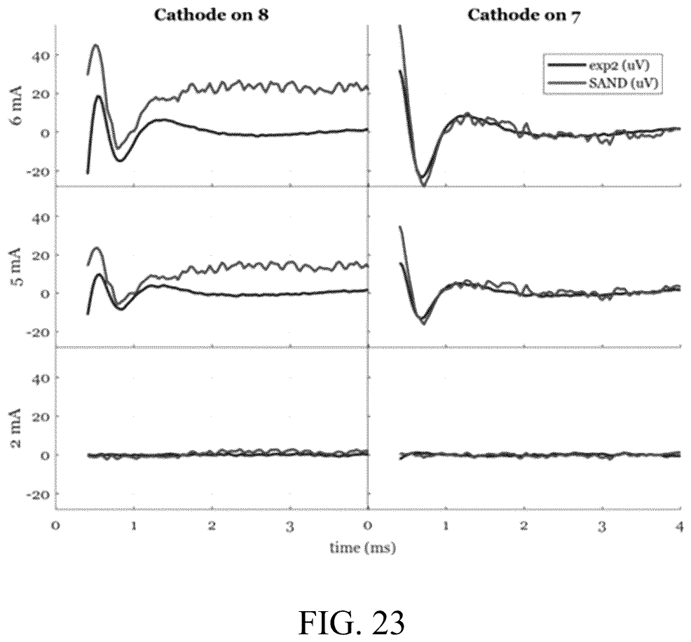

Many different waveforms are used to remove stimulus artifacts in the decaying phase, a double exponential fit (exp2), a linear and exponential fit (lin-exp), and polynomial functions (poly). While each can be effective at removing artifacts, they may not be able to model all the complexities of the linear responses following stimulation, and they are computationally expensive to implement. To compare exp2 and SAND, an example of an ECAP is shown in FIG. 23 where there is a negative bump shown in the exp-2 fit 2 ms after the stimulus artifact. This negative bump is common across all stimulation amplitudes but with varying sizes on stimulation with Cathode 7 &8. The SAND method successfully eliminates this bump on Cathode 8, producing a cleaner ECAP signal. However, it can be seen that the signal using SAND is not as clean as using exp2 because the noise on the stimulus waveform from the lower stimulation amplitude is amplified, which results in more noise in the final result.

FIG. 23 shows a stimulus artifact using the double-exponential method (exp2, blue lines) versus the novel SAND method (red lines) on Ch(11)−Ch(12) in participant #2's first visit. The two columns represent the two stimulation polarities. The rows represent stimulation amplitudes (suprathreshold: 6 and 5 mA; and sub-threshold: 2 mA). The SAND method used the 1-mA stimulation response to remove the artifacts for all stimulation amplitudes.

A Comparison of Dose Response Curves of ECAPs Across Electrodes within Subject with Different Methods

ECAPs show a thresholded response, below the stimulation threshold an ECAP is not seen, and above the threshold the ECAP grows with stimulation amplitude. For comparison, the ECAP amplitude is measured in two ways, the peak-to-peak (P2P) amplitude between the N1 and P2 peak, and the area under the curve (AUC). While P2P and AUC capture ECAP amplitude, AUC also captures additional information about peak widths and is not dependent on the presence of true peaks. With both P2P and AUC measurements, the ECAP amplitudes were near zero below the threshold and increase linearly above it, as seen in FIG. 24. The two methods for removing the stimulus artifact, exp2 and SAND are also compared. With the combination of stimulus artifact removal and the ECAP amplitude measurements, all the dose-response curves were similar. SAND appeared to be a bit noisier in the estimations at subthreshold levels when compared to the more stable estimates from exp2. This variation can be attributed to the specific noise characteristics associated with the subthreshold response scaling in SAND.

The consistency in ECAP thresholds across different electrodes is also seen in FIG. 24, where the thresholds appear almost identical regardless of the electrode used. The response gain seems to be different depending on the distance from the stimulation electrodes. Stimulation was applied through electrodes 7&8, and nearby electrodes, like channels 4&5, showed higher gains as compared to those further away, such as 1&2, demonstrating the spatial sensitivity of ECAP responses to the stimulation site. There is also larger stimulation artifact contamination at more proximal sites.

FIG. 24 shows dose-response curves of multiple channels in one participant (#3, visit 2) for cathode-on-8 stimulation. The inset shows the definition of ECAP metrics: peak-to-peak (P2P) and area under the curve (AUC). The dose response curves include P2P (top row) and AUC (bottom row) when artifact removal was done using the double exponential curve fit (exp2, left column) versus the novel SAND method (right column). The channels shown here are the good channels with ECAPs in this dataset, determined by an investigator. The SAND method used the 1-mA stimulation response to remove the artifacts for all stimulation amplitudes.

ECAP dose response curves across patients.

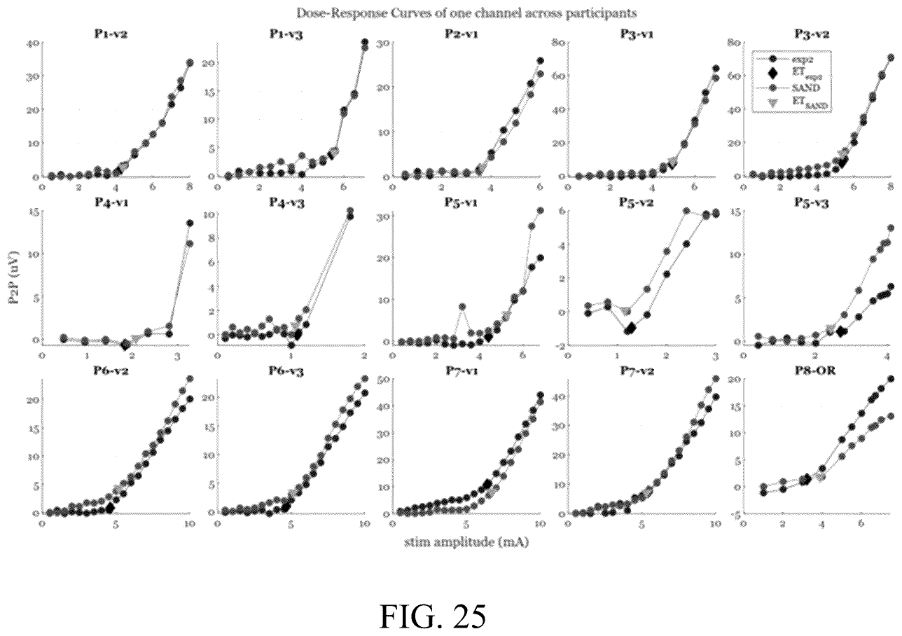

FIG. 25 shows the dose-response curve of the ECAPs from electrodes 2&3, for 8 patients, many of which are recorded for multiple visits. Most notably, the threshold response is remarkably consistent across different patients, indicating a robust and generalizable characteristic of the ECAP response.

Both the SAND and exp2 methods yielded comparable results in terms of threshold determination across all patients. The similarities in thresholds determined by these two methods further reinforce the reliability and accuracy of our SAND approach in estimating ECAP thresholds.

However, within the same patient across different visits, there are notable variations in both threshold and gain. This is particularly evident in patients P1 between visits 2 and 3, and P5 across all visits 1, 2, and 3. These changes can probably be largely attributed to variations in posture during the ECAP measurements at different visits. For example, some patients who initially sat during the first visit opted to lay down in subsequent visits for greater comfort. This change in posture can have a pronounced impact on ECAP measurements due to the alteration in the positioning of the spinal cord and the electrodes.

Patients 4 and 5, who had electrodes placed in the cervical or upper-thoracic area, displayed more variability in their response. This increased variability is likely due to the heightened sensitivity of the spinal cord in these areas to subtle changes in field strengths, which can be brought about by even minor positional adjustments.

FIG. 25 shows peak-to-peak (P2P) dose-response curves of Ch(2)−Ch(3) in all participants (P in subplot titles) and visits (v-x in subplot titles) during cathode-on-8 stimulation. The blue curves represent ECAPs processed using the double exponential (exp2) artifact removal method, while the red curves are for the SAND method. ECAP thresholds (ET) are shown as black diamonds for exp2 and orange triangles for SAND. The SAND method used the 1-mA stimulation response to remove the artifacts for all stimulation amplitudes.

How welldoes the ECAP threshold represent the sensory perception threshold?

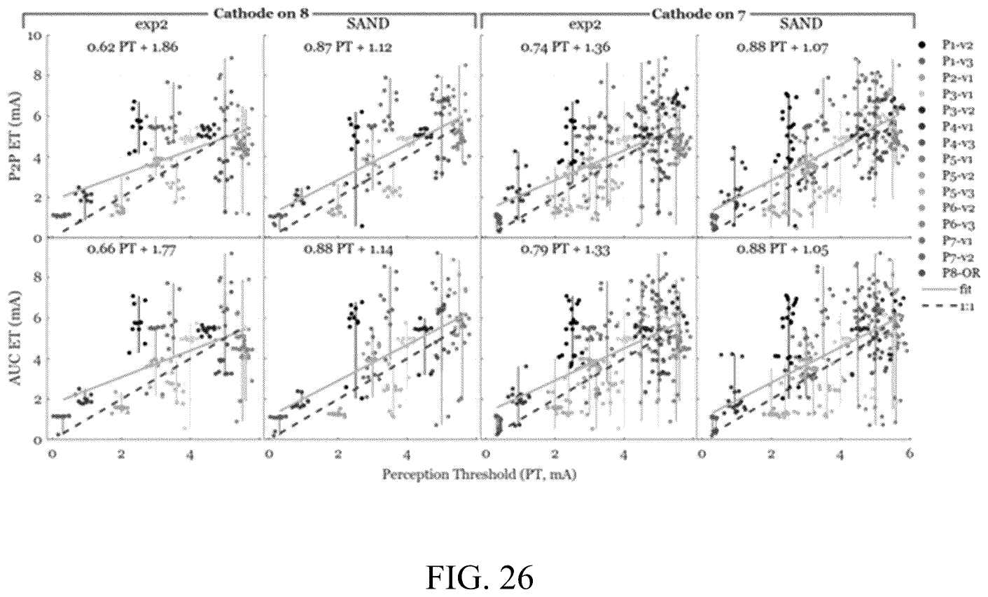

FIG. 26 presents a scatter plot of ECAP threshold vs perception thresholds across subjects with different methods of measuring the ECAP. The ECAPs measured using P2P and AUC from data cleaned using exp2 and SAND were compared. The results from cathode leading on electrode 8 vs on electrode 7 are shown. The multiple data points of the same color correspond to different channels within the same visit.

To estimate the perception threshold, subjects were asked to report on their perception as the stimulation amplitude was gradually increased, while the ECAPs were simultaneously measured. This enabled a comparison between the estimated ECAP threshold across all electrodes and the perception threshold.

For most subjects, each ECAP was compared to its threshold for each stimulus polarity (channel 7 or 8 as cathodic leading). However, for subjects 1, 2, and 8 (recorded in the OR), there was only one threshold available, which was used for both recordings. In general, within a visit, electrodes exhibited similar thresholds, as indicated by groups of dots with the same color on the y-axis in FIG. 7. Nevertheless, there were instances at the perception threshold where some electrodes were above threshold while others were below, particularly noticeable in the graphs with cathode on Ch 7, which had more subthreshold responses.

Supporting the observations from FIG. 23, changes in thresholds across visits were also observed, as seen in subject 5's visits. In general, there was strong association between ECAP threshold and patient perception thresholds, both within patients across visits and across different patients. To measure this association, a line was fitted to the amplitude of the ECAP as a function of the perception threshold. Ideally, this line would align with the 1:1 dashed reference line. However, the fitted lines were generally above the 1:1 line, suggesting that the ECAP threshold tends to be slightly higher than the perception thresholds. While most ECAP thresholds were above the perception threshold, there were instances where ECAPs were detected below the perception threshold. The convergence of the fit lines to the 1-1 at higher amplitudes indicates that patients with higher perception thresholds tend to have ECAP thresholds that are more accurate with respect to perception. Conversely, the most sensitive subjects often perceived stimulation before ECAPs were detectable.

Overall, the differences between the exp2 and SAND methods in detecting ECAP relative to perception were minimal, underscoring the effectiveness of both approaches in this context.

FIG. 26 shows on average the ECAP threshold (ET) is greater than the perception threshold (PT). ETs were calculated using peak-to-peak (P2P, top row) or area-under-the curve (AUC, bottom row). The columns are the combination of the two stimulation polarities (cathode on 8 and cathode on 7) and the two artifact-removal methods (exp2 and SAND). The dots' colors represent a participant/visit combination. The ETs of the same color are for different channels (see FIG. 5); and the corresponding PT is randomly scattered in x-axis (using MATLAB's swarmchart) around the patient reported value, depicted as a vertical line with the same color where the line spans the whole range of detected ETs on the y-axis. The gray lines are the best-fit lines to the data with coefficients shown in each subplot. The dashed line is a visual reference line with 1:1 correspondence between the two axes.

### Comparing Correlation Between Different Estimates of ECAP Threshold and Perception.

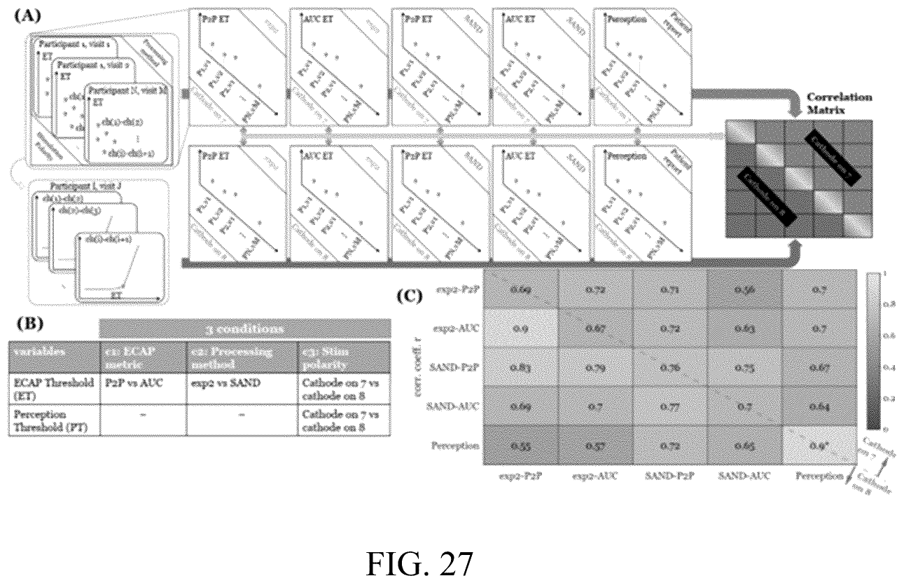

In the endeavor to determine the most accurate method for measuring ET, from the possible combinations of exp2 or SAND with P2P or AUC, and compared the resulting ETs to the measured PTs. This was done for two stimulation conditions, Ch 7 and Ch 8 cathodic stimulation. The Pearson cross-correlation (R) between the estimated ET and the reported PT is shown for all combinations of these measures as a matrix in FIG. 27.

The matrix shown in FIG. 27 illustrates the correlation between thresholds measured using the different methods. Along the diagonal of the matrix is the correlation between ETs obtained from stimulation with flipped polarity (Ch 7 vs. Ch 8 as the cathode). Off diagonal is the correlation between the different methods. The strongest correlation for ET between polarities was for SAND-P2P, with a R=0.76. The correlation in perception thresholds from 7 vs 8 as cathode was even higher, reaching R=0.9. The variation in ET between the polarities is likely attributable to the relative position of the recording electrodes to the cathode, which impacts the sensitivity in detecting ECAPs. However, the actual response, as gauged by perception, appears less affected by the cathode's location.

Below the diagonal is the correlation Ch 8 as cathode and above the diagonal is for Ch 7 cathode. Crucially, the correlation between perception threshold and ECAP amplitudes was consistently higher for SAND than exp2, particularly for the channel 7 cathode, and comparable for the channel 8 cathode. This suggests that SAND more accurately reflects perception compared to exp2. To determine if SAND has a significantly higher correlation with perception than exp2, correlation with perception using both stimulation polarities was measured. The correlation between SAND and perception R=0.7, N=315, for exp2 and perception R=0.63, N=316. By performing a Fisher Z-transform to the R values and applying a Z-test p=0.12 was found, so the null hypothesis could not be rejected. So, it was concluded that exp2 and SAND are equivalent, but it is believed that the data is trending towards significance and more data would support the conclusion that SAND is superior.

FIG. 27 shows the correlation matrix among the ECAP thresholds (ET) and participants' reported perception thresholds (PT). FIG. 27(A) shows an illustration of the data used to construct the correlation matrix. FIG. 27(B) shows the ETs were determined under three conditions: (c1) the ECAP metric being either peak-to-peak (P2P ET) or area under the curve (AUC ET); (c2) the artifact removal method being either double exponential (exp2) or SAND; and (c3) the stimulation polarity being either cathode on 8 or cathode on 7. FIG. 27(C) shows the correlation matrix. The upper diagonal contains correlations between pairs of conditions c1 and c2 (e.g., P2P ET of exp2 and P2P ET of SAND) while fixing condition c3 to cathode on 7. The lower triangle is similarly structured but with fixing condition c3 to cathode on 8. The diagonal contains correlations between pairs of condition c3 while fixing conditions c1 and c2. All correlations have p<1E-13. *The correlation between PTs under the two stimulation polarities excluded data from patients 1, 2 and 8, where PT was measured for a stimulation train switching polarity per pulse. Hence, no polarity-wise PT was available.

### Computational Efficiency of Stimulation Artifact Removal Comparison

In evaluating the computational efficiency of stimulation artifact removal methods, the computation time of each algorithm was measured to estimate their power costs. This assessment is particularly important for potential real-time applications on implantable devices with low power budgets. Although fitting a model to data and subtracting the resulting curve is computationally intensive, the actual computation time on modern desktop computers is generally negligible. However, understanding the power demands of each algorithm is crucial for their application in implantable medical devices.

Table 5 presents the execution times for both the SAND and exp2 methods in Matlab as a proxy for power consumption. The computation time required to fit data in a window sampled at 24 kHz for about 4 msec was calculated. Remarkably, the SAND method is approximately 10,000 times faster than the exp2 method. This reduction in computational cost not only highlights the efficiency of SAND but also makes it a viable candidate for implementation in implantable devices.

This analysis, conducted using MATLAB's timeit function on an Intel Core i7-5820K processor, was further substantiated by a one-sided 2-sample t-test with unequal variance, confirming the significant difference in computation times between the two methods. The computational efficiency of SAND, paired with its accuracy as previously discussed, underscores its potential as a preferred method for stimulation artifact removal in clinical and research applications involving implantable devices.

Table 5 shows the execution time of the two artifact removal methods in seconds, using MATLAB's timeit function on an Intel Core i7-5820K. A one-sided 2-sample t-test with unequal variance was used to get the p-value.

TABLE 5
Execution Time of Artifact Removal Methods

| Exec. time (s) | exp2 | SAND | p-value |
|---|---|---|---|
| Mean | 3.5E−2 | 3.1E−6 | <1E−6 |
| Median | 3.4E−2 | 3.2E−6 | NA |
| Interquartile | 3.0E−2-3.7E−2 | 2.6E−6-3.3E−6 | NA |
| Range | 1.5E−2-9.0E−2 | 1.9E−6-9.9E−6 | NA |

### Discussion

ESR triggered by electrical stimulation during SCS is prone to artifact contamination if the recording contacts are within close proximity of the stimulation contacts. Because components of the ECAP can appear within 1 to 2 ms, it is often contaminated by the stimulation capacitive discharges. Thus, stimulation artifact removal is often the first and necessary step to measuring ECAPs from the ESR. A method to remove the possible stimulation artifact contamination is to fit an exponential, or polynomial, based curve immediately following the stimulation and then subtracting the resulting fit. Curve fitting is computationally expensive, which may have a significant impact on overall energy consumption if implemented on an embedded platform of an implantable pulse generator (IPG). In addition, even a double exponential fit (exp2) may not be able to model all linear responses after stimulation. Here these findings test a new method for stimulation artifact removal for estimation of ECAPS called (S)AND.

In this disclosure several innovations have been introduced. The first is (S)AND, for removing stimulus artifacts. The second is an efficient method for measuring ECAP thresholds (ET). The third is a validation of the ET against patient reported PT to identify which method of calculating ECAP amplitude is most accurate.

The SAND method removes artifacts first by measuring the difference in signal responses between neighboring electrodes, for common mode rejection, and second by scaling the subthreshold signals by the stimulation amplitude and subtracting from the scaled suprathreshold signals. This has been reported in the literature as a zero-amplitude response template or scaled amplitude response. This approach has the advantage that it does not require curve fitting and it can account for complex waveforms that scale linearly with amplitude. This method utilizes the fact that the stimulation artifacts scale linearly with stimulation amplitude (FIG. 21C) while neural signals do not. Thus, these differences effectively remove the linear artifacts while retaining any nonlinear neural responses. However, it should be noted that the resulting differential evoked response is the change of evoked responses between the two locations in space and stimulation amplitudes, and is not the absolute evoked responses caused by a single stimulation channel (FIG. 22).

SAND was compared to the exp2 curve fit, which were used in previous publications. The estimation of the amplitude of the ECAP using the area under the curve (AUC) vs measuring the difference between the first negative peak to the second positive peak was compared. Using the four combinations of SAND/exp2 and AUC/P2P to compare dose response curves and ECAP thresholds (ET). To determine which method is most accurate, the correlation of the estimated ET was calculated to the measured patient reported PT.

When comparing ECAPS estimated using exp2 and SAND, resulting curves were found to be quite similar (see FIG. 23). However, SAND is less prone to pseudo responses that may be produced from curve fitting and not from a neural source.

A similar approach to SAND is to flip the polarity of stimulation on each stimulation and summing the ESR. It has been found that flipping is effective at removing stimulation artifacts. However, the timing and amplitude of the ECAP can change with each polarity, probably by activating the spinal cord at slightly different locations, so when summing the resulting waveform is not a reflection of the evoked response at either polarity. Therefore, polarity flipping and summing have not been pursued and have found SAND to be more accurate in reflecting the underlying neuronal response.

SAND uses a spatial difference to reject common mode signals, but this too can introduce errors. For a traveling ECAP in the spinal cord, the arrival of the ECAP at each electrode may be slightly delayed, but the responses are overlapping. The spatial derivative results in a different ECAP waveform than would be expected to be measured using a unipolar electrode in the spinal cord with a distal reference. However, the resulting waveform refines the onset of the event in time, so this is not an acceptable change to the waveform.

It has been found that the dose response curves, also known as growth curves, look similar whether removing the stimulus artifact using SAND or the exp2 methods or measuring the size of the response using peak-to-peak or AUC.

An efficient method has been developed for identifying the ECAP threshold by fitting two lines, one for subthreshold data and one for suprathreshold data, which intersect at the threshold and finding the threshold that minimizes the fit error. This method is robust and efficient. A brute force method was used, measuring the error by testing the threshold at all amplitudes and selecting the best one, but this easily could be made more efficient using algorithms to test a subset and following a gradient descent of the error.

In general, patients reported a lower paresthesia threshold when compared to ECAP thresholds (FIG. 24C, FIG. 25). This observation is consistent with previous studies where researchers reported paresthesia threshold is generally lower than the threshold of evoked responses. However, a greater discrepancy was seen for subjects with low amplitude thresholds than high amplitude thresholds. Importantly, this implies that the ECAP-based closed-loop stimulation mechanism cannot achieve paresthesia-free therapy.

Next, a correlation analysis was ran between the reported paresthesia and detected evoked response thresholds for all patients and sessions. The results show that using SAND together with peak-to-peak amplitude as the feature has the highest correlation with paresthesia threshold, followed by the SAND with the AUC feature, which were comparable (FIG. 26). While correlation between SAND and PT was not significantly higher than exp2 and PT given the size of our data set, the results are trending and it is believed that with more data it would be shown that SAND is superior.

Most importantly, for an embedded system, it was found that SAND is much more computationally efficient than exp2 for removing stimulation artifacts. At the sampling rates and window lengths used, SAND took 1/10000 the computation time than exp2.

### Disadvantages of SAND

There are some disadvantages to SAND. The first is that some of the stimulation times are being used to estimate artifact rather than being therapeutic, which may reduce the efficacy of the therapy. This probably isn't a big problem because most spinal cord stimulation therapies are delivered with stim on for 1 minute and 5 minutes off. For comfort, stimulation is often ramped up at the beginning of stimulation. The stimulation artifact may be estimated from the pulses during the ramp up to the therapeutic stimulation amplitude. Alternatively, a relatively low percentage of stimulation trials may be used as catch trials to estimate the stimulus artifact without significant decrease in total stimulation delivered.

SAND results in a slightly noisier estimate of the ECAP. Because SAND uses a scaled subthreshold response the noise in the low amplitude response is scaled when subtracting from the super-threshold response, which increases the noise in the estimated ECAP. A fit function does not introduce noise and therefore is cleaner. This might be mitigated by averaging several sub-threshold responses together before subtracting.

### Conclusions

SAND is efficient, it is less prone to artifacts caused by fitting functions to data. ECAPS estimated with SAND have higher correlations with patient perception thresholds. Generally, ECAPS have higher thresholds than perception, therefore closed-loop adjustment of ECAP amplitude is probably limited to stimulation that causes paresthesia.

### Example 3

Example 3 provides a method and system for closed-loop neuromodulation to restore function after spinal cord injury.

Spinal cord stimulation (“SCS”) is clinically approved only for the treatment of pain disorders. However, this example provides a revolutionary use of neuromodulation of the spinal cord stem to treat paralysis after spinal cord injury (“SCI”).

Currently, the method for adapting spinal cord stimulation to individual patients is ad hoc, and efforts to optimize electrode selection and stimulus waveform selection have not been robust. In the clinical world, closed-loop neuromodulation of spinal cord stimulation using Evoked Compound Action Potentials (“ECAPs”) has shown potential, especially in the context of spinal cord injury, where injury patterns can be heterogeneous, and patient adaptation is crucial for restoring function.

Previously, there were no systematic ways of tailoring SCS for SCI, since the current method for treating pain using ECAPS only uses peak to peak amplitude approaches. This fails to consider the novel results that come from these neural circuits at higher amplitudes because they do not apply to the treatment of chronic pain. This disclosure specifically extends the use of a novel feature of ECAPS in order to tailor therapy as well as a method to do so that is specific to each segmental circuit providing the customization necessary for patients with heterogeneous injury patterns and states of function.

It has been discovered that through neuromodulation, evoked compound action potentials (“ECAPs”) coming from the spinal cord can be measured, which is currently used in closed-loop clinical neuromodulation paradigms. This discovery suggests that sufficient stimulation effectively provides volitional control in patients with spinal cord injuries. For example, it was discovered that if stimulation is provided beyond what the typical range of ECAPs give for pain, changes in the ECAP waveform that correlate with muscle activity will be observed. Thus, the range that corresponds to the potential modulation or restoration of movement can be rapidly identified in each subject.

Additionally, this method of stimulating and recording could also be used to restore autonomic functions dynamically in patients with spinal cord injury. A comprehensive system has been developed, which includes a data analysis pipeline and regularization algorithms that can play a crucial role in this discovery. This system enables the identification of specific signals and the cleaning of data, leading to the dissection of a significant biomarker. This biomarker's statistical significance for the recovery of function is determined through the innovative methods herein. To aid in understanding, informative visual representations have been generated, including plots.

In some cases, there are mechanistic directions for SCS in SCI, which can include acute and chronic effects, motor and autonomic effects, bidirectional translational opportunities, combined neuromodulation approaches, replication of effects, and segmental vs. supraspinal.

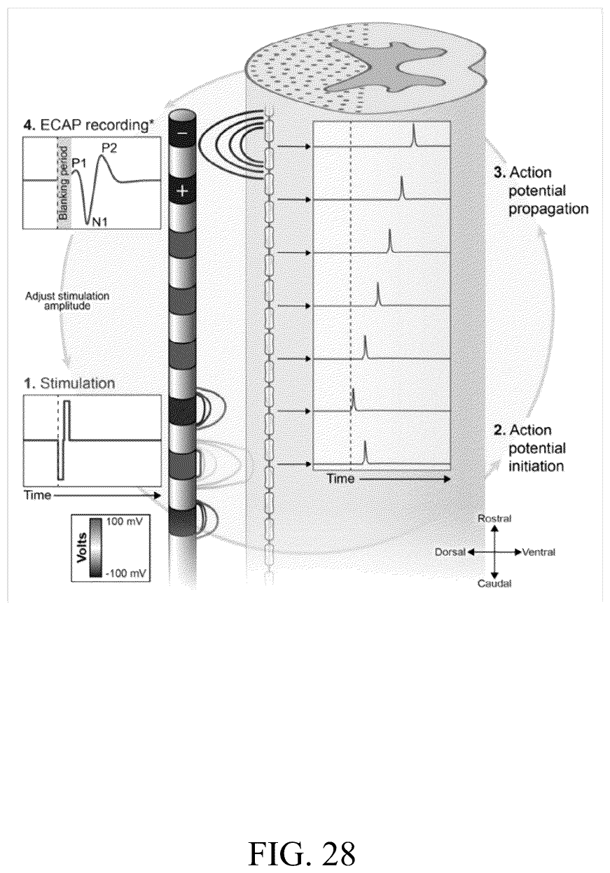

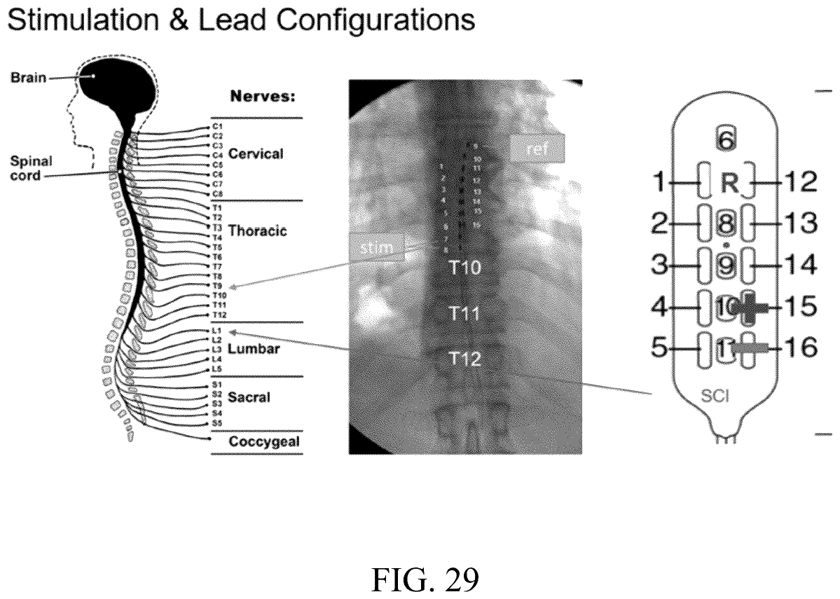

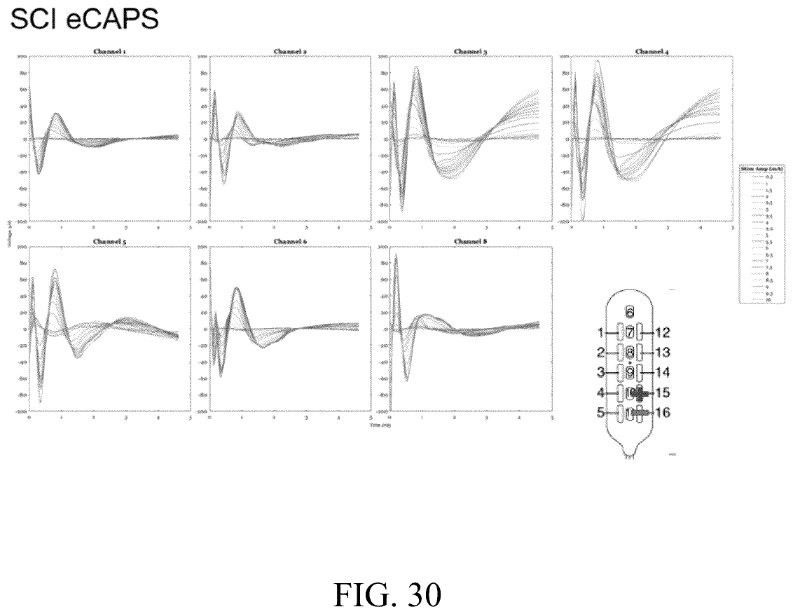

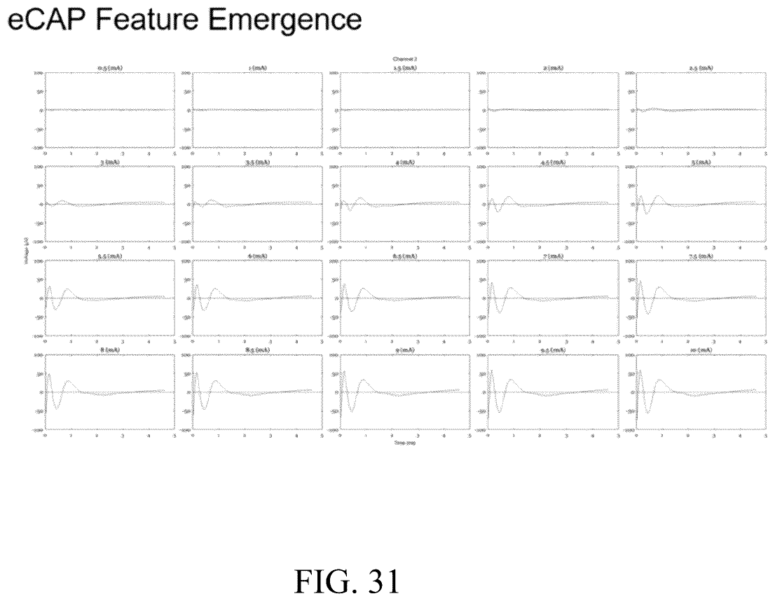

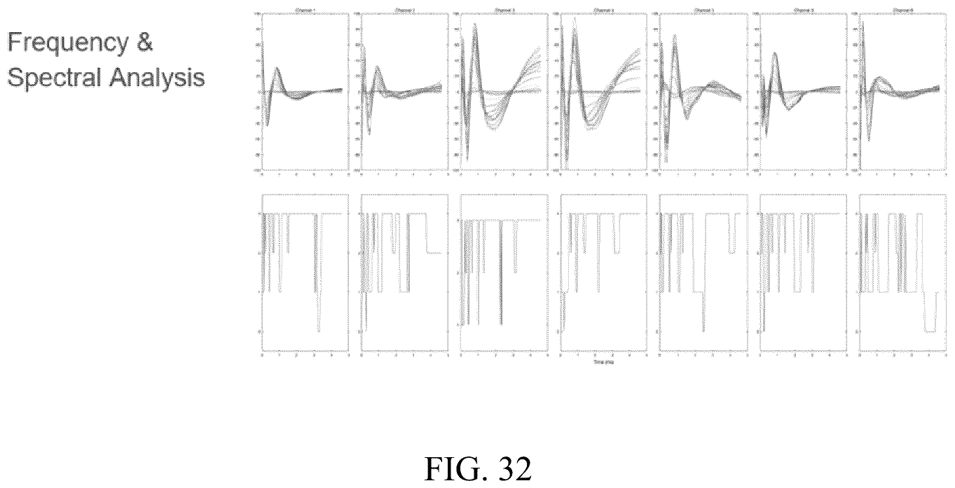

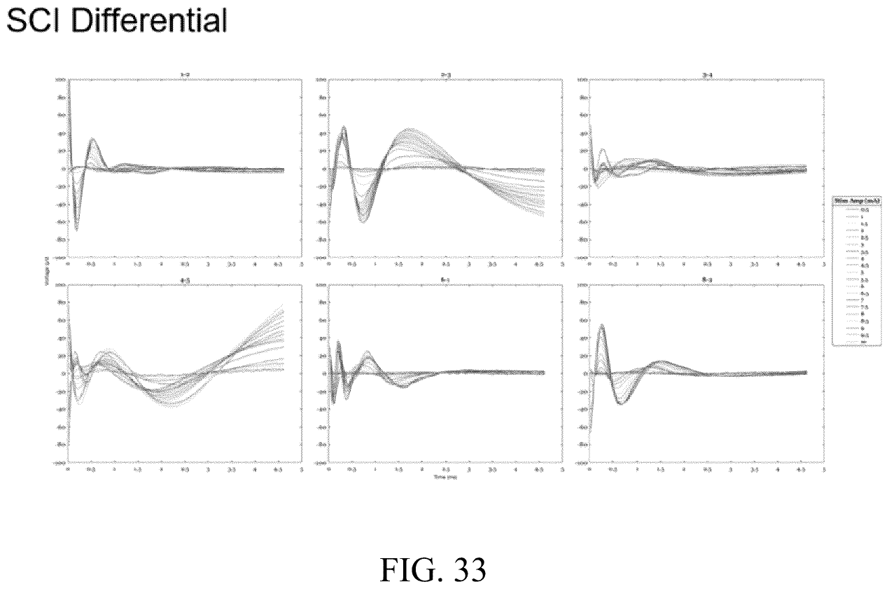

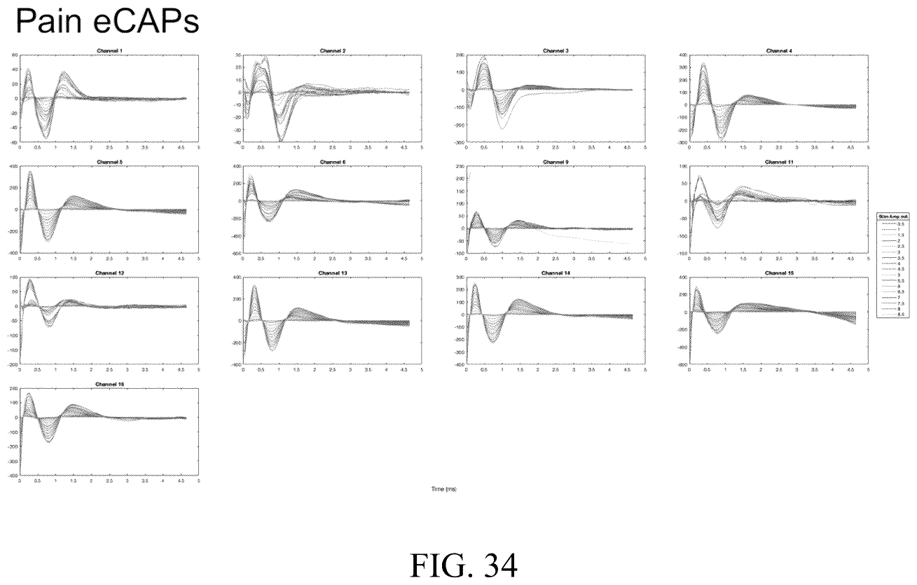

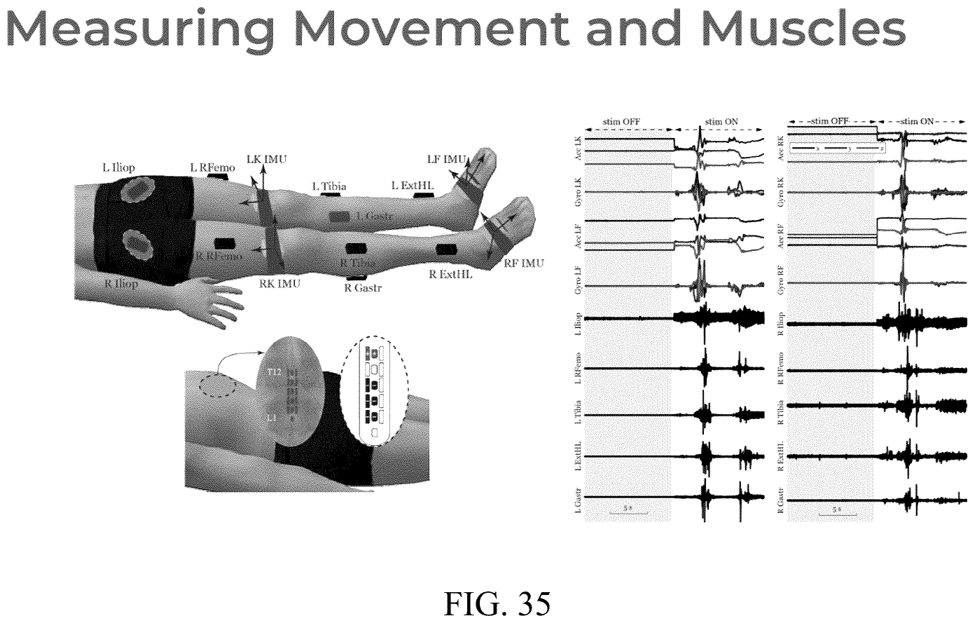

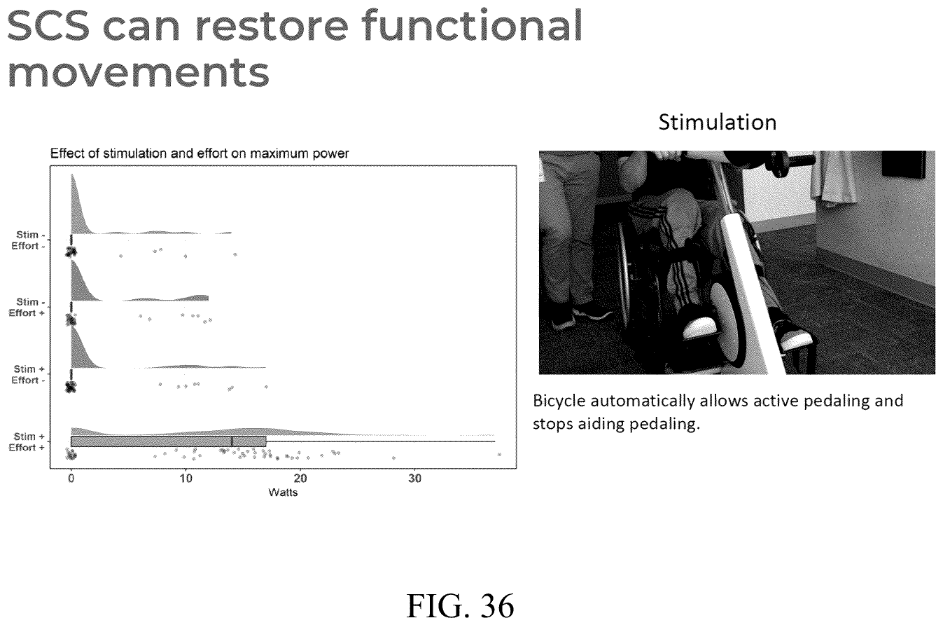

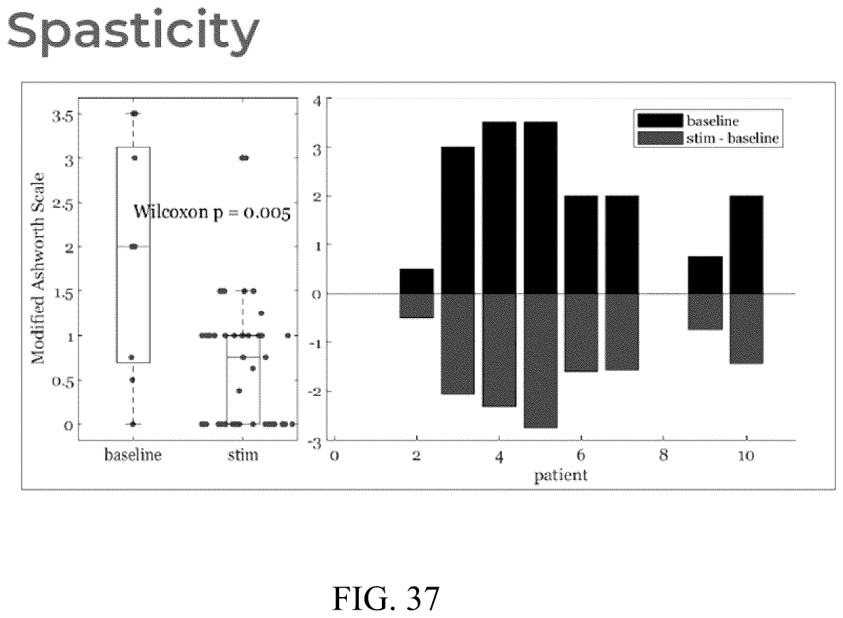

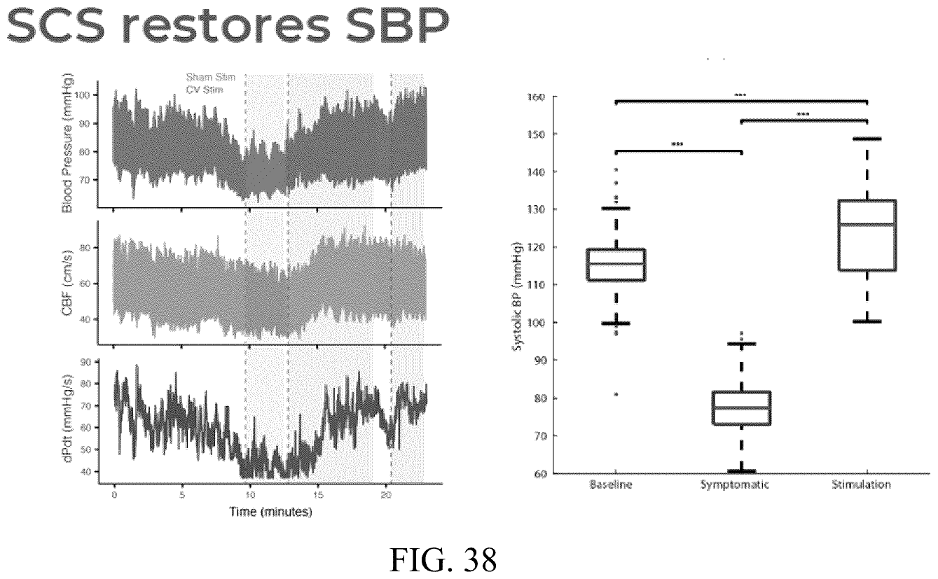

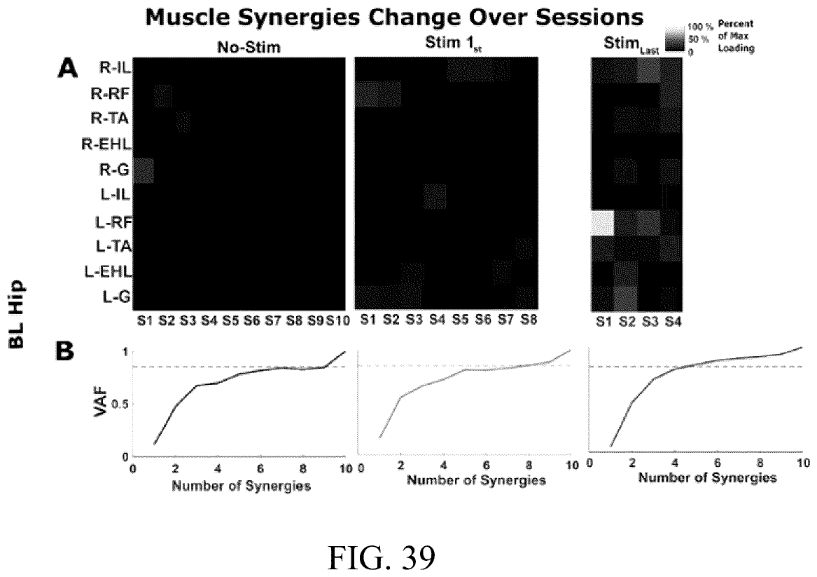

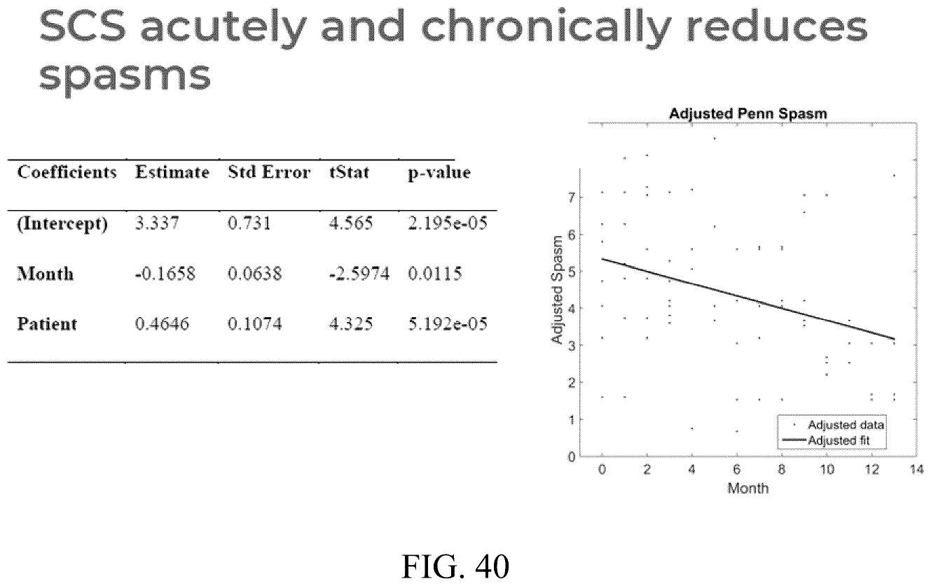

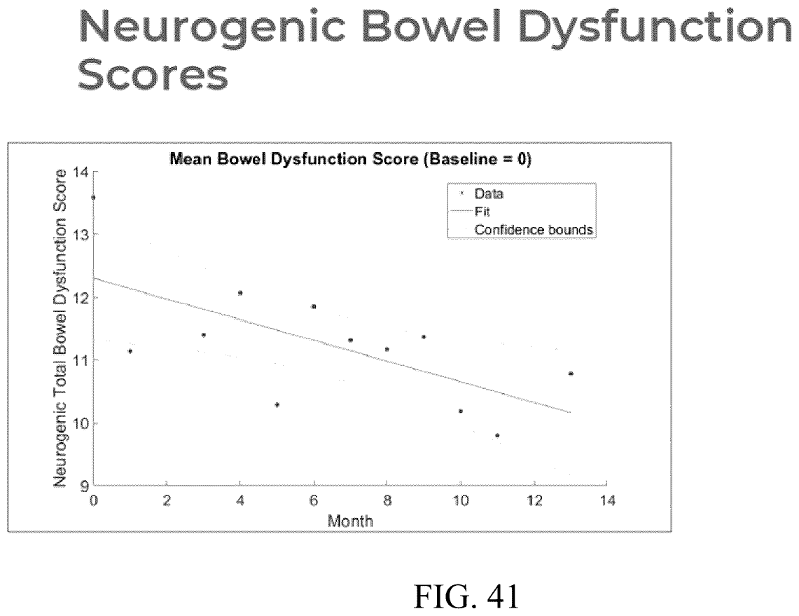

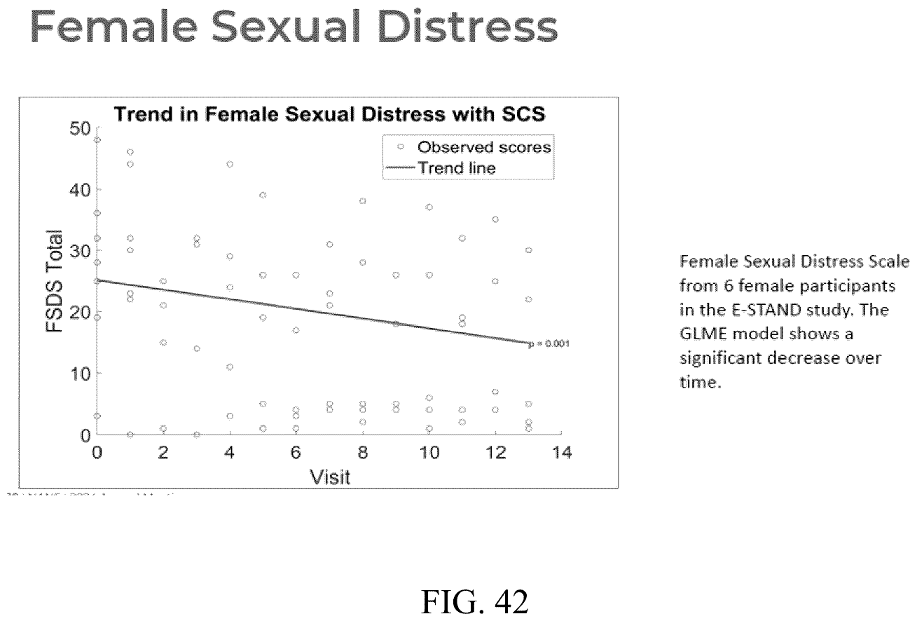

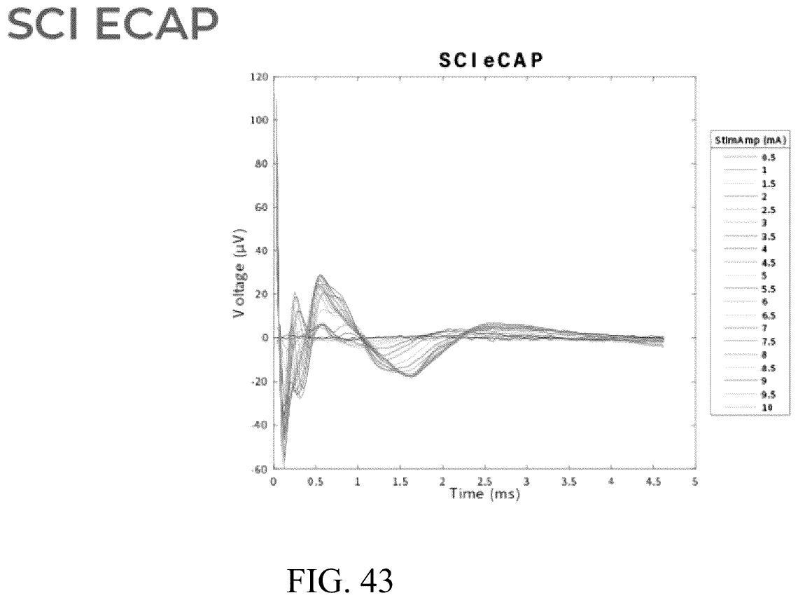

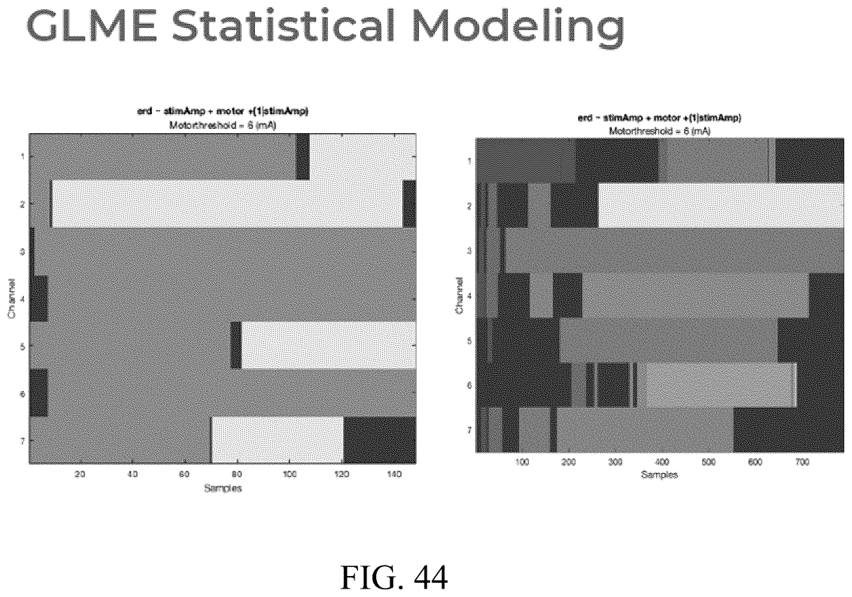

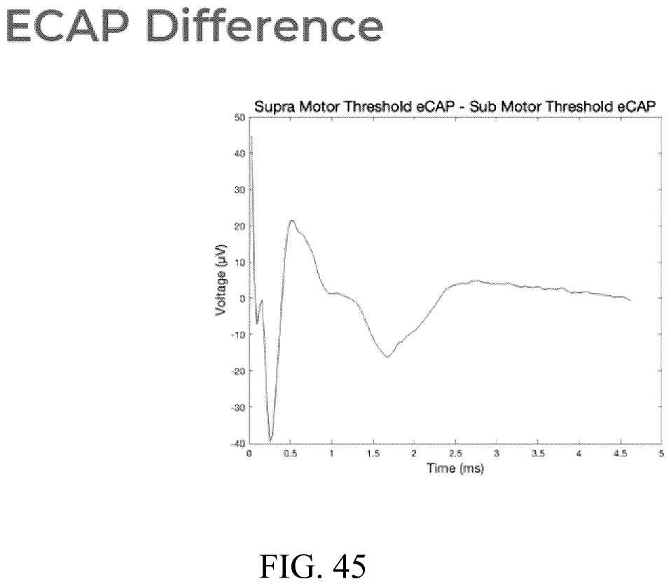

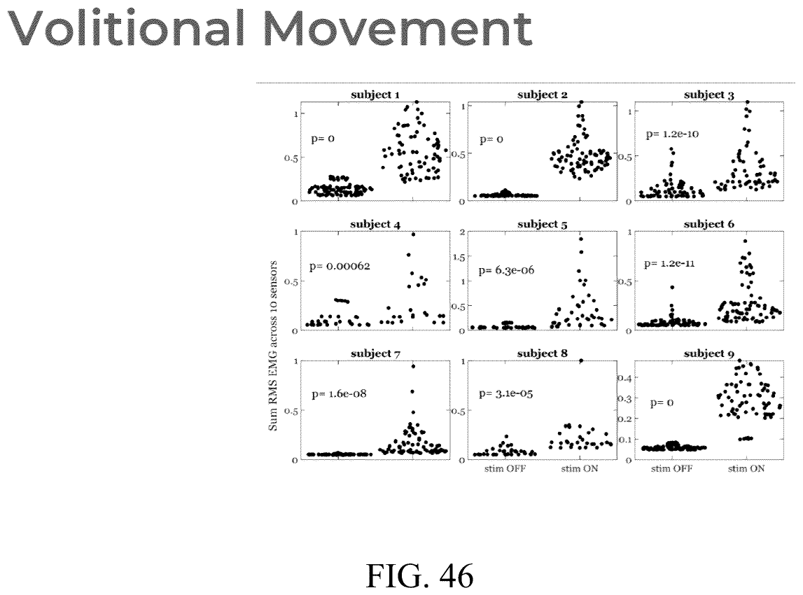

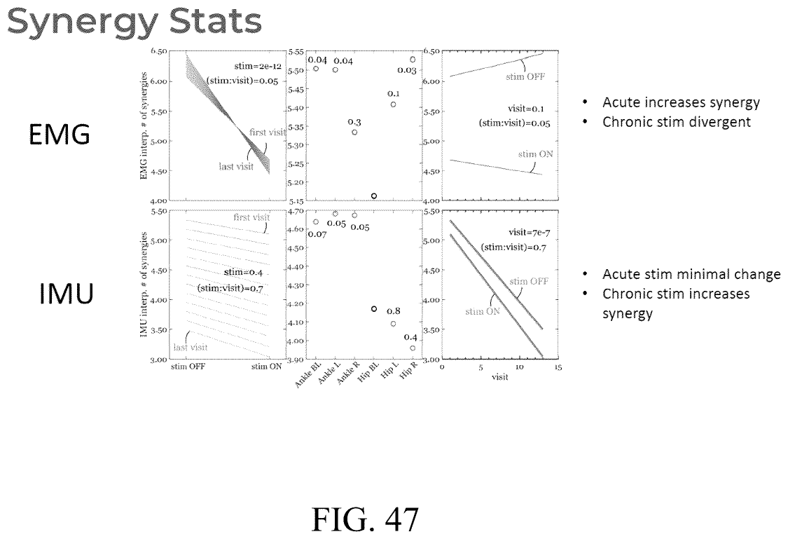

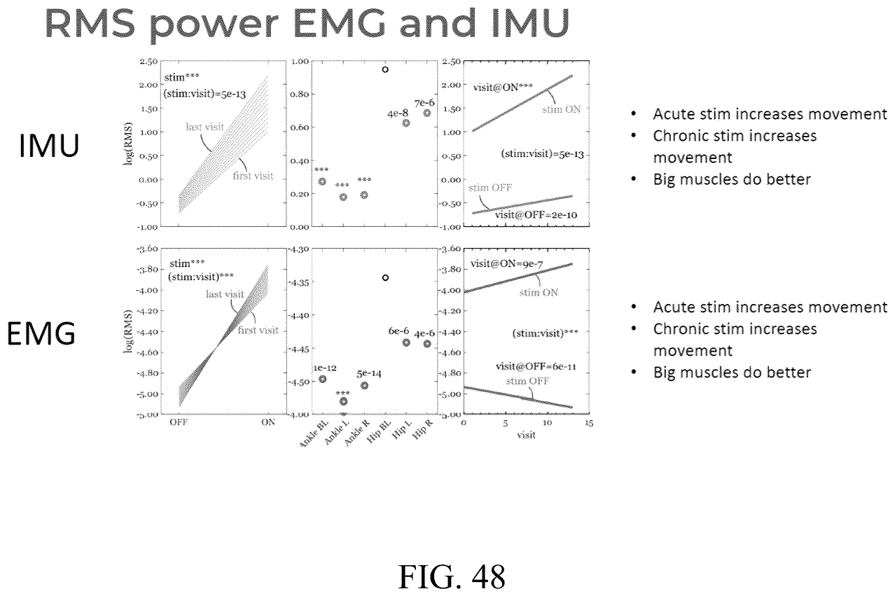

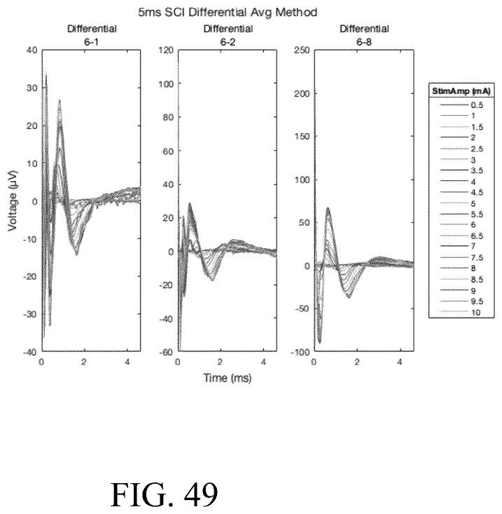

FIGS. 28-49 illustrate additional examples in accordance with some embodiments described in the present disclosure. For example, FIG. 28 illustrates an example of how ECAPs can be captured on a SCS lead, FIG. 29 illustrates an example of SCS lead placement for chronic pain vs SCI and stimulation configuration on a paddle lead, and FIG. 30 illustrates an example of ECAP responses across stimulation amplitude recorded on a paddle lead placed in the lumbar spinal cord for SCI. FIG. 31 illustrates an example of how ECAP morphology and features, from a single channel, emerge across stimulation amplitude, and FIG. 32 illustrates an example of ECAP responses across time and statistically significant clusters of time identifying morphology/time windows of interest. FIG. 33 illustrates an example of bipolar subtracted referenced ECAP responses from an SCS paddle, with the title indicating which electrodes were referenced. FIG. 34 illustrates an example of ECAP responses across stimulation amplitude recorded on leads placed in the thoracic spinal cord for chronic pain. FIG. 35 illustrates an example of an electromyography (EMG) and accelerometer recording setup and how muscle activity and volitional movement improves with spinal cord stimulation in a participant receiving SCS for SCI. FIG. 36 illustrates an example illustrating the effect of SCS and effort on maximal force on a volitional biking task. FIG. 37 illustrates an example of how SCS impacts spasticity, quantified by the Modified Ashworth Scale, over time. FIG. 38 illustrates an example of how SCS impacts systolic blood pressure, FIG. 39 illustrates an example of how SCS impacts muscle synergies, FIG. 40 illustrates an example of how SCS reduces the number of spasms over time, FIG. 41 illustrates an example of how SCS improves bowel function over time, and FIG. 42 illustrates an example of how SCS improves female sexual function over time. FIG. 43 illustrates an example ECAP response across increasing stimulation amplitudes of lumbar SCS in a participant with SCI. FIG. 44 illustrates an example of statistically significant timepoints on ECAP waveform accounting for different features in a generalized linear mixed-effects (GLME) model. FIG. 45 illustrates an example of resulting waveforms when ECAPs above and below the motor threshold are subtracted from each other. FIG. 46 illustrates an example of overall EMG power improving with SCS turned on, and FIG. 47 illustrates examples of how EMG and IMU metrics capture different components of muscle synergies improvement with SCS. FIG. 48 illustrates examples of how EMG and IMU metrics capture different components of muscle power improvement with SCS. FIG. 49 illustrates an example of ECAP response across increasing stimulation amplitudes of lumbar SCS in a participant with SCI.

The present disclosure has described one or more preferred embodiments, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular embodiments or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or embodiments. Further, references to particular rotational or other movements (e.g., counterclockwise rotation) is generally intended as a description only of movement relative a reference frame of a particular example of illustration.

In some embodiments, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, embodiments of the disclosure can be implemented as a set of instructions, tangibly embodied on a non-transitory computer-readable media, such that a processor device can implement the instructions based upon reading the instructions from the computer-readable media. Some embodiments of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use embodiments of the disclosure. Various modifications to the illustrated examples will be readily apparent to those skilled in the art, and the generic principles herein can be applied to other examples and applications without departing from the principles disclosed herein. Thus, embodiments of the disclosure are not intended to be limited to embodiments shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, +10%, +5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

Various features and advantages of the disclosure are set forth in the following claims.

What is claimed is:

1. A neurostimulation system comprising:
a plurality of stimulation electrodes;
a plurality of recording electrodes;
a waveform generator in electrical communication with the plurality of stimulation electrodes;
one or more computing devices in electrical communication with the plurality of recording electrodes and the waveform generator, the one or more computing devices being configured to:
cause the waveform generator to electrically excite the plurality of stimulation electrodes of the plurality of stimulation electrodes;
after electrically exciting the plurality of stimulation electrodes, receive, from the plurality of recording electrodes, a neural signal;
determine that the neural signal is an electrically evoked compound action potential (“ECAP”) by:
determining one or more features of the neural signal; and
determining that the neural signal is an ECAP based on the one or more features of the neural signal;
determine one or more stimulation parameters, based on one or more characteristics of the ECAP; and
cause the waveform generator to electrically excite the plurality of stimulation electrodes according to the one or more stimulation parameters.

2. The neurostimulation system of claim 1, wherein the one or more features of the neural signal does not include a peak to peak amplitude of the neural signal.

3. The neurostimulation system of claim 1, wherein the one or more features of the neural signal includes:
an area under the curve (AUC) of the neural signal;
an average slope of the neural signal;
an average voltage of the neural signal; or
a latency of a negative peak of the neural signal.

4. The neurostimulation system of claim 1, wherein the one or more features of the neural signal includes an area under the curve (AUC) of the neural signal.

5. The neurostimulation system of claim 1, wherein determining that the neural signal is an ECAP includes the one or more computing devices:
comparing a feature of the one or more features to a predefined threshold of the feature; and
based on the feature being greater than or under the predefined threshold, determining that the neural signal is an ECAP.

6. The neurostimulation system of claim 5, wherein the predefined threshold is determined by the one or more computing devices by:
receiving neural signal data;
generating feature data by extracting features from the neural signals in the neural signal data;
generating classification data by classifying the neural signals in the neural signal data based on the feature data;
training a machine learning model on training data comprising pairs of matched classification data and neural signal data; and
receiving, from the machine learning model, the predefined threshold corresponding to the feature.

7. The neurostimulation system of claim 5, wherein determining the predefined threshold for each feature includes the one or more computing devices assessing an accuracy of each feature for a first classification between ECAPs and non-ECAPs and a second classification between ECAPs and outliers.

8. The neurostimulation system of claim 7, wherein assessing the accuracy of each feature includes the one or more computing devices using a receiver operating characteristic (“ROC”) analysis for each feature.

9. The neurostimulation system of claim 1, wherein determining that the neural signal is an ECAP includes the one or more computing devices:
providing the neural signal to a machine learning model that classifies the neural signal as an ECAP, a non-ECAP, or an outlier, the machine learning model being trained on training data comprising neural signals classified as ECAP, neural signals classified as non-ECAP, and neural signals classified as outliers;
receiving, from the machine learning model, an output classifying the neural signal as an ECAP; and
determining that the neural signal is an ECAP based on the output from the machine learning model classifying the neural signal as an ECAP.

10. The neurostimulation system of claim 9 wherein the machine learning model is a K-Nearest Neighbors (KNN) classifier.

11. The neurostimulation system of claim 1, wherein determining that the neural signal is an ECAP includes the one or more computing devices:
determining a peak to peak amplitude (P2P) of the neural signal;
determining that the neural signal has at least one peak; and
determining that the neural signal is an ECAP, based on the determination that the neural signal has the at least one peak and the P2P exceeds a P2P threshold, wherein the P2P threshold is 15 μV.

12. The neurostimulation system of claim 1, wherein determining that the neural signal is an ECAP includes the one or more computing devices:
receiving, from a patient, a user input indicative of a perception threshold of stimulation; and
determining that the neural signal is an ECAP, based on a stimulation amplitude used to electrically excite the plurality of stimulation electrodes being at or above the perception threshold.

13. The neurostimulation system of claim 1, wherein determining that the neural signal is an ECAP includes the one or more computing devices:
determining that the neural signal is not a non-ECAP, not an outlier, or both; and
based on determining that the neural signal is not a non-ECAP, not an outlier, or both, determining that the neural signal is an ECAP.

14. The neurostimulation system of claim 1, wherein the neural signal is a first neural signal;
wherein causing the waveform generator to electrically excite the plurality of stimulation electrodes uses a first polarity of the plurality of stimulation electrodes; and
wherein the one or more computing devices are further configured to:
reverse the polarity of the plurality of stimulation electrodes to a second polarity opposite of the first polarity;
cause the waveform generator to electrically excite the plurality of stimulation electrodes with the second polarity;
after electrically exciting the plurality of stimulation electrodes with the second polarity, receive, from at least two recording electrodes of the plurality of recording electrodes, a second neural signal;
add the first neural to the second neural signal to derive a clean ECAP.

15. A computer implemented method for classifying neural signals, the method comprising:
providing, using one or more computing devices, a neural signal to a machine learning model that classifies the neural signal as an ECAP, a non-ECAP, or an outlier, the machine learning model being trained on training data comprising neural signals classified as ECAP, neural signals classified as non-ECAP, and neural signals classified as outliers;
receiving, from the machine learning model and using the one or more computing devices, an output classifying the neural signal as an ECAP; and
determining, using the one or more computing devices, that the neural signal is an ECAP based on the output from the machine learning model classifying the neural signal as an ECAP.

16. The computer implemented method of claim 15, wherein the machine learning model is trained on ECAP neural signals, non-ECAP neural signals, and outlier neural signals cleaned from artifacts.

17. The computer implemented method of claim 16, wherein each of the neural signals of the training data is a raw neural signal having been cleaned; and
wherein each clean raw neural signal is cleaned by subtracting each raw neural signal by a different neural signal to yield the cleaned neural signal.

18. The computer implemented method of claim 17, wherein cleaning the artifacts include the one or more computing devices:
providing a first neural signal resulting from a first stimulation amplitude;
providing a second neural signal resulting from a second stimulation amplitude;
normalizing the first neural signal by the first stimulation amplitude to yield a first normalized neural signal;
normalizing the second neural signal by the second stimulation amplitude to yield a second normalized neural signal;
subtracting the first normalized neural signal from the second normalized neural signal to yield the a clean neural signal.

19. The computer implemented method of claim 16, wherein each of the neural signals of the training data is a raw neural signal having been cleaned;
wherein each cleaned neural signal is cleaned by the one or more computing devices:
fitting a curve to each raw neural signal;
subtracting the curve from each raw neural signal to yield a cleaned neural signal.

20. The computer implemented method of claim 19, wherein the curve includes an exponential function, a polynomial curve, a single exponential function, a double exponential function, or a second order polynomial function, or a linear exponential fit.

21. A neurostimulation system comprising:
a plurality of stimulation electrodes;
a plurality of recording electrodes;
a waveform generator in electrical communication with the plurality of stimulation electrodes;
one or more computing devices in electrical communication with the plurality of recording electrodes, the one or more computing devices being configured to:
cause the waveform generator to electrically excite two stimulation electrodes of the plurality of stimulation electrodes;
after electrically exciting the two stimulation electrodes, receive, from at least two recording electrodes of the plurality of recording electrodes, a neural signal;
determine that the neural signal is an electrically evoked compound action potential (“ECAP”);
remove a stimulation artifact from the neural signal that is an ECAP to yield a clean neural signal;
determine one or more stimulation parameters, based on one or more characteristics of the clean neural signal; and
cause the waveform generator to electrically excite the stimulation electrodes according to the one or more stimulation parameters.

22. The neurostimulation system of claim 21, wherein the cleaned neural signal is cleaned by subtracting the neural signal by a stimulation artifact.

23. The neurostimulation system of claim 22, wherein the neural signal is a third neural signal, and wherein the stimulation artifact is determined by the one or more computing devices:
providing a first neural signal resulting from a first stimulation amplitude;
providing a second neural signal resulting from a second stimulation amplitude;
normalizing the first neural signal by the first stimulation amplitude to yield a first normalized neural signal;
normalizing the second neural signal by the second stimulation amplitude to yield a second normalized neural signal;
subtracting the first normalized neural signal from the second normalized neural signal to yield the stimulation artifact.

24. A method of training a machine learning model, the method comprising:
receiving neural signal data;
generating feature data by extracting features from the neural signals in the neural signal data;
generating classification data by classifying the neural signals in the neural signal data based on the feature data; and
training a machine learning model on training data comprising pairs of matched classification data and neural signal data.

25. The method of claim 24, wherein generating the feature data comprises clustering the features.

26. The method of claim 25, wherein the features are clustered using k-means clustering.

27. The method of claim 24, wherein extracting the features comprises performing a principal component analysis (PCA) on the neural signals to generate principal components and storing the principal components as the features.

28. The method of claim 27, wherein storing the storing the principal components comprises storing a first number of the principal components.
